(19) United States
(12) Patent Application Publication
(10) Pub. No.: US 2025/0306023 A1
(43) Pub. Date: Oct. 2, 2025

(54) METHODS AND SYSTEMS OF PREDICTING MULTISYSTEM INFLAMMATORY SYNDROME IN CHILDREN (MIS-C)

(71) Applicants: University of Connecticut, Farmington, CT (US); Connecticut Children's Medical Center, Hartford, CT (US); New York State Department of Health, Albany, NY (US)

(72) Inventors: Michele Maltz-Matyschsyk, South Windsor, CT (US); Clare Melchiorre, Vernon, CT (US); Michael A. Lynes, Eastford, CT (US); Juan C. Salazar, Glastonbury, CT (US); David A. Lawrence, Slingerlands, NY (US); Aishwarya Jadhav, Schenectady, NY (US); Katherine Herbst, Hartford, CT (US)

(21) Appl. No.: 19/093,637

(22) Filed: Mar. 28, 2025

Related U.S. Application Data
(60) Provisional application No. 63/570,852, filed on Mar. 28, 2024.

(51) Int. Cl. G01N 33/569; C12Q 1/689; G01N 33/543; G01N 33/68

(52) U.S. Cl. CPC G01N 33/56983; C12Q 1/689; G01N 33/54386; G01N 33/6863; G01N 2333/165; G01N 2469/20; G01N 2470/04

(57) ABSTRACT

A method and system for predicting/diagnosing inflammatory diseases, such as, multisystem inflammatory syndrome in children (MIS-C), Kawasaki disease, Severe Acute Respiratory Syndrome Coronavirus 2 (SARS-CoV-2), respiratory syncytial virus, an adenovirus, influenza A, B C, or D, and/or parainfluenza in a subject is described.

### RELATED APPLICATION INFORMATION

This application claims priority to U.S. Application No. 63/570,852, filed on Mar. 28, 2024, the contents of which are herein incorporated by reference.

### GOVERNMENT SUPPORT

This invention was made with government support under HD105613 awarded by the National Institutes of Health. The government has certain rights in the invention.

### FIELD OF THE DISCLOSURE

Described herein are the methods and systems for assessing, namely, predicting or diagnosing inflammatory diseases, such as, multisystem inflammatory syndrome in children (MIS-C), Kawasaki disease, Severe Acute Respiratory Syndrome Coronavirus 2 (SARS-CoV-2), respiratory syncytial virus, an adenovirus, influenza A, B C, or D, and/or parainfluenza in a subject.

### BACKGROUND

Acute pediatric SARS-CoV-2 infections, although usually mild, have hospitalized over 150,000 children during the coronavirus disease-2019 (COVID-19) pandemic. In rare instances, two to six weeks after an acute infection, children can develop a severe inflammatory disorder known as multisystem inflammatory syndrome in children (MIS-C). The syndrome is non-specific and is associated with, but not limited to, the following symptoms: abdominal pain, diarrhea, vomiting, rashes, red eyes, red or swollen hands/feet, red cracked lips, cough, sore throat, fever, cardiovascular dysfunction, and respiratory dysfunction. According to the CDC as of March 2025, in the U.S. there have been 9,769 MIS-C patients. Half of children with MIS-C are admitted to the intensive care unit (ICU) and 80 children have died from MIS-C. Unfortunately, MIS-C symptoms and the associated immune response mimic other inflammatory diseases. For example, Kawasaki disease (an acute and self-limited vasculitis of unknown etiology) shares many clinical and laboratory markers with MIS-C (e.g., fever, rash, red eyes, inflammatory markers) leading to misdiagnosis and delaying definitive management. Therefore, there is an immediate need for novel methods and systems for effective prediction/diagnosis (detection) of MIS-C in a subject.

### SUMMARY

In one embodiment, the present disclosure relates to a biomarker panel for assessing a subject suspected of having or having multisystem inflammatory syndrome in children (MIS-C) or Kawasaki disease, the panel comprising a plurality of first biomarker detection agents, wherein each first biomarker detection agent specifically binds to a corresponding target biomarker, wherein the target biomarkers comprise: Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), and immunoregulatory alpha globulin (IRA).

In some aspects, in the biomarker panel, the target biomarkers further comprise one or more of: B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof.

In some aspects, in the biomarker panel, the target biomarkers further comprise one or more of: Marapsin, fractalkine (CX3CL1), C-C chemokine ligand 20 (CCL20), interleukin-21, C-C chemokine ligand 17 (CCL17), interleukin-1β (IL-1β), or any combination thereof.

In another embodiment, the present disclosure relates to a biomarker panel for assessing a subject suspected of having or having multisystem inflammatory syndrome in children (MIS-C) or Severe Acute Respiratory Syndrome Coronavirus 2 (SARS-CoV-2), the panel comprising a plurality of first biomarker detection agents, wherein each first biomarker detection agent specifically binds to a corresponding target biomarker, wherein the target biomarkers comprise: Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), and interleukin-23 (IL-23).

In some aspects, in the biomarker panel, the target biomarkers further comprise one or more of: Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7), or any combination thereof.

In some aspects, in the biomarker panel, the target biomarkers further comprise one or more of: Pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2), or any combination thereof.

In yet another embodiment, the present disclosure relates to a biomarker panel for assessing a subject suspected of having or having multisystem inflammatory syndrome in children (MIS-C) or respiratory syncytial virus (RSV), the panel comprising a plurality of first biomarker detection agents, wherein each first biomarker detection agent specifically binds to a corresponding target biomarker, wherein the target biomarkers comprise: Granulocyte-Macrophage Colony-Stimulating Factor (GM-CSF), Pentraxin-3 (PTX-3), Marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23).

In some aspects, in the biomarker panel, the target biomarkers further comprise one or more of: Perforin (Perf), C-X-C motif chemokine ligand 11 (CXCL11), Epidermal Growth Factor (EGF), interleukin-10 (IL-10), interleukin-8 (IL-8), interleukin-21 (IL-21), interleukin-2 (IL-2), immunoregulatory alpha globulin (IRA), or any combination thereof.

In some aspects, in the biomarker panel, the target biomarkers further comprise one or more of: Granzyme B (GRAND B), fractalkine (CX3CL1), C-C motif chemokine ligand 24 (CCL24), interleukin-7 (IL-7), C-C chemokine ligand 20 (CCL20), interleukin-6 (IL-6), C-C chemokine ligand 17 (CCL17), interleukin-4 (IL-4), interleukin-17 (IL-17), interleukin-15 (IL-15), tumor necrosis factor alpha (TNFα), interleukin-1β (IL-1β), interferon gamma (INFγ), interleukin-12 (IL-12), or any combination thereof.

In some aspects of any of the above-described biomarker panels, each first biomarker detection agent is an antibody or antibody binding fragment thereof. Specifically, each antibody or antibody binding fragment thereof is immobilized on a solid support. Moreover, in some aspects, the solid support is a particle, microparticle, bead, plate, well, or chip. Additionally, in some aspects, each first biomarker detection agent is labeled with a detectable label.

In further aspects of any of the above-described biomarker panels, the panel is configured as a multiplex assay or as an ELISA assay.

In another embodiment, the present disclosure relates to a kit for assessing a subject suspected of having or having multisystem inflammatory syndrome in children (MIS-C) or Kawasaki disease, comprising:
a. a plurality of first biomarker detection agents, wherein each first biomarker detection agent specifically binds to a corresponding target biomarker, wherein the target biomarkers comprise: Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), and immunoregulatory alpha globulin (IRA); and
b. instructions for use of the kit.

In some aspects of the above kit, the target biomarkers comprise one or more of: B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof.

In further aspects of the above kit, the target biomarkers further comprise one or more of: Marapsin, fractalkine (CX3CL1), C-C chemokine ligand 20 (CCL20), interleukin-21, C-C chemokine ligand 17 (CCL17), interleukin-1β (IL-1β), or any combination thereof.

In yet another embodiment, the present disclosure relates to a kit for assessing a subject suspected of having or having multisystem inflammatory syndrome in children (MIS-C) or Severe Acute Respiratory Syndrome Coronavirus 2 (SARS-CoV-2), comprising:
a. a plurality of first biomarker detection agents, wherein each first biomarker detection agent specifically binds to a corresponding target biomarker, wherein the target biomarkers comprise: Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), and interleukin-23 (IL-23); and
b. instructions for use of the kit.

In some aspects of the above kit, the target biomarkers comprise one or more of: Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7), or any combination thereof.

In some aspects of the above kit, the target biomarkers comprise one or more of: Pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2), or any combination thereof.

In yet another embodiment, the present disclosure relates to a kit for assessing a subject suspected of having or having multisystem inflammatory syndrome in children (MIS-C) or respiratory syncytial virus (RSV), comprising:
a. a plurality of first biomarker detection agents, wherein each first biomarker detection agent specifically binds to a corresponding target biomarker, wherein the target biomarkers comprise: Granulocyte-Macrophage Colony-Stimulating Factor (GM-CSF), Pentraxin-3 (PTX-3), Marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23); and
b. instructions for use of the kit.

In some embodiments of the above kit, the target biomarkers comprise one or more of: Perforin (Perf), C-X-C motif chemokine ligand 11 (CXCL11), Epidermal Growth Factor (EGF), interleukin-10 (IL-10), interleukin-8 (IL-8), interleukin-21 (IL-21), interleukin-2 (IL-2), immunoregulatory alpha globulin (IRA), or any combination thereof.

In further embodiments of the above kit, the target biomarkers comprise one or more of: Granzyme B (GRAND B), fractalkine (CX3CL1), C-C motif chemokine ligand 24 (CCL24), interleukin-7 (IL-7), C-C chemokine ligand 20 (CCL20), interleukin-6 (IL-6), C-C chemokine ligand 17 (CCL17), interleukin-4 (IL-4), interleukin-17 (IL-17), interleukin-15 (IL-15), tumor necrosis factor alpha (TNFα), interleukin-1β (IL-1β), interferon gamma (INFγ), interleukin-12 (IL-12), or any combination thereof.

In still further embodiments, in any of the above-described kits, the kit further comprises a plurality of second biomarker detection agents which specifically bind to the corresponding target biomarker. Specifically, in such kits, each first biomarker detection agent, each second biomarker detection agent, or each first biomarker detection agent and each second biomarker detection agent is an antibody or antibody binding fragment thereof. Moreover, in any of the above-described kits, each of the first biomarker detection reagents is immobilized on a solid support. In some aspects, the solid support is a particle, microparticle, bead, plate, well, or chip. Moreover, in any of the above-described kits, each first biomarker detection reagent is labeled with a detectable label, or the second biomarker detection reagent is labeled with a detectable label.

In another embodiment, the present disclosure relates to a method for assessing a subject suspected of having or having multisystem inflammatory syndrome in children (MIS-C) or Kawasaki disease, the method comprising:
a. determining a measurement of a panel of first target biomarkers in a sample obtained from a subject suspected of having multisystem inflammatory syndrome in children (MIS-C) or Kawasaki disease, wherein: (i) the measurement comprises determining an amount of each of a plurality of first target biomarkers in the panel; and (ii) the target biomarkers comprise: Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), and immunoregulatory alpha globulin (IRA);
b. calculating a relative intensity value for each first target biomarker in the panel based on the measurements in step a;
c. comparing the relative intensity values calculated in step b) to reference values for the target biomarkers; and
d. assessing the subject for MIS-C or Kawasaki disease based on the comparison made in step c.

In some aspects of the above method, the first target biomarkers further comprise one or more of: B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof.

In some aspects of the above method, the panel further comprises a plurality of second target biomarkers, wherein the second target biomarkers comprise one or more of: Marapsin, fractalkine (CX3CL1), C-C chemokine ligand 20 (CCL20), interleukin-21, C-C chemokine ligand 17 (CCL17), interleukin-1β (IL-1β), or any combination thereof.

In some aspects of the above method, the method further comprises:
a. determining a measurement of each of the first target biomarkers and second target biomarkers in a sample obtained from a subject suspected of having multisystem inflammatory syndrome in children (MIS-C) or Kawasaki disease;
b. calculating a relative intensity value for each first target biomarker and each second target biomarker in the panel based on the measurements in step a;
c. comparing the relative intensity values calculated in step b) to reference values for the target first and second biomarkers; and
d. assessing the subject for MIS-C or Kawasaki disease based on the comparison made in step c.

In yet another embodiment, the present disclosure relates to a method for assessing a subject suspected of having or having multisystem inflammatory syndrome in children (MIS-C) or Severe Acute Respiratory Syndrome Coronavirus 2 (SARS-CoV-2), the method comprising:
a. determining a measurement of a panel of first target biomarkers in a sample obtained from a subject suspected of having multisystem inflammatory syndrome in children (MIS-C) or Severe Acute Respiratory Syndrome Coronavirus 2 (SARS-CoV-2), wherein: (i) the measurement comprises determining an amount of each of a plurality of first target biomarkers in the panel; and (ii) the target biomarkers comprise: Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), and interleukin-23 (IL-23);
b. calculating a relative intensity value for each first target biomarker in the panel based on the measurements in step a;
c. comparing the relative intensity values calculated in step b) to reference values for the target biomarkers; and
d. assessing the subject for MIS-C or Severe Acute Respiratory Syndrome Coronavirus 2 (SARS-CoV-2) based on the comparison made in step c.

In some aspects of the above method, the first target biomarkers further comprise one or more of: Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7), or any combination thereof.

In some aspects of the above method, the first target biomarkers further comprise one or more of: B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof.

In some aspects of the above method, the panel further comprises a plurality of second target biomarkers, wherein the second target biomarkers comprise one or more of: Pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2), or any combination thereof.

In some aspects of the above method, the method further comprises:
a. determining a measurement of each of the first target biomarkers and second target biomarkers in a sample obtained from a subject suspected of having multisystem inflammatory syndrome in children (MIS-C) or SARS-CoV-2;
b. calculating a relative intensity value for each first target biomarker and each second target biomarker in the panel based on the measurements in step a;
c. comparing the relative intensity values calculated in step b) to reference values for the target first and second biomarkers; and
d. assessing the subject for MIS-C or SARS-CoV-2 based on the comparison made in step c.

In yet another embodiment, the present disclosure relates to a method for assessing a subject suspected of having or having multisystem inflammatory syndrome in children (MIS-C) or respiratory syncytial virus, the method comprising:
a. determining a measurement of a panel of first target biomarkers in a sample obtained from a subject suspected of having multisystem inflammatory syndrome in children (MIS-C) or respiratory syncytial virus, wherein: (i) the measurement comprises determining an amount of each of a plurality of first target biomarkers in the panel; and (ii) the target biomarkers comprise: Granulocyte-Macrophage Colony-Stimulating Factor (GM-CSF), Pentraxin-3 (PTX-3), marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23);
b. calculating a relative intensity value for each first target biomarker in the panel based on the measurements in step a;
c. comparing the relative intensity values calculated in step b) to reference values for the target biomarkers; and
d. assessing the subject for MIS-C or respiratory syncytial virus based on the comparison made in step c.

In some aspects of the above method, the first target biomarkers further comprise one or more of: Perforin (Perf), C-X-C motif chemokine ligand 11 (CXCL11), Epidermal Growth Factor (EGF), interleukin-10 (IL-10), interleukin-8 (IL-8), interleukin-21 (IL-21), interleukin-2 (IL-2), immunoregulatory alpha globulin (IRA), or any combination thereof.

In some aspects of the above method, the panel further comprises a plurality of second target biomarkers, wherein the second target biomarkers comprise one or more of: Granzyme B (GRAND B), fractalkine (CX3CL1), C-C motif chemokine ligand 24 (CCL24), interleukin-7 (IL-7), C-C chemokine ligand 20 (CCL20), interleukin-6 (IL-6), C-C chemokine ligand 17 (CCL17), interleukin-4 (IL-4), interleukin-17 (IL-17), interleukin-15 (IL-15), tumor necrosis factor alpha (TNFα), interleukin-1β (IL-1β), interferon gamma (INFγ), interleukin-12 (IL-12), or any combination thereof.

In some aspects of the above method, the method further comprises:
a. determining a measurement of each of the first target biomarkers and second target biomarkers in a sample obtained from a subject suspected of having multisystem inflammatory syndrome in children (MIS-C) or respiratory syncytial virus;
b. calculating a relative intensity value for each first target biomarker and each second target biomarker in the panel based on the measurements in step a;
c. comparing the relative intensity values calculated in step b) to reference values for the target first and second biomarkers; and
d. assessing the subject for MIS-C or respiratory syncytial virus based on the comparison made in step c.

In yet another embodiment, the present disclosure relates to a device for assessing a subject suspected of having or having multisystem inflammatory syndrome in children (MIS-C) or Kawasaki disease, where the device comprises:
a. a measuring unit for determining a relative intensity of each of a plurality of first target biomarkers in a sample obtained from a subject suspected of having MIS-C or Kawasaki disease, wherein the measuring unit comprises a detection system for detecting and measuring an amount of each of the plurality of first target biomarkers and further wherein the first target biomarkers comprise: Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), and immunoregulatory alpha globulin (IRA); and
b. an evaluation unit operably linked to the measuring unit comprising a database with stored reference values for the first target biomarkers and a data processor comprising instructions for carrying out a comparison of the relative intensity of each first target biomarker to reference values for the target biomarkers and assessing the subject based on the comparison, said evaluation unit being capable of automatically receiving the relative intensity values for each of the biomarkers from the measuring unit.

In one aspect of the device, the first target biomarkers further comprise one or more of B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof.

In one aspect of the device:
a. the measuring unit determines a relative intensity of each of a plurality of first target biomarkers and second target biomarkers in the sample, wherein the measuring unit comprises a detection system for detecting and measuring an amount of each of the plurality of first target biomarkers and second target biomarkers and further wherein the second target biomarkers comprise one or more of marapsin, fractalkine (CX3CL1), C-C chemokine ligand 20 (CCL20), interleukin-21, C-C chemokine ligand 17 (CCL17), interleukin-1β (IL-1β), or any combination thereof; and
b. the evaluation unit comprises a database with stored reference values for the first target biomarkers and second target biomarkers and a data processor comprising instructions for carrying out a comparison of the relative intensity of each first target biomarker and each second target biomarker to reference values for the first and second target biomarkers and assessing the subject based on the comparison.

In another embodiment, the present disclosure relates to a device for assessing a subject suspected of having or having multisystem inflammatory syndrome in children (MIS-C) or Severe Acute Respiratory Syndrome Coronavirus 2 (SARS-CoV-2), where the devices comprises:
a. a measuring unit for determining a relative intensity of each of a plurality of first target biomarkers in a sample obtained from a subject suspected of having MIS-C or Severe Acute Respiratory Syndrome Coronavirus 2 (SARS-CoV-2), wherein the measuring unit comprises a detection system for detecting and measuring an amount of each of the plurality of first target biomarkers and further wherein the first target biomarkers comprise: Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), marapsin, interleukin-2 receptor alpha chain (CD25), Heat Shock Protein 70 (HSP70), and interleukin-23 (IL-23); and
b. an evaluation unit operably linked to the measuring unit comprising a database with stored reference values for the first target biomarkers and a data processor comprising instructions for carrying out a comparison of the relative intensity of each first target biomarker to reference values for the target biomarkers and assessing the subject based on the comparison, said evaluation unit being capable of automatically receiving the relative intensity values for each of the biomarkers from the measuring unit.

In one aspect of the device, the first target biomarkers further comprise one or more of interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7), or any combination thereof.

In one aspect of the device:
a. the measuring unit determines a relative intensity of each of a plurality of first target biomarkers and second target biomarkers in the sample, wherein the measuring unit comprises a detection system for detecting and measuring an amount of each of the plurality of first target biomarkers and second target biomarkers and further wherein the second target biomarkers comprise one or more of pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2); and
b. the evaluation unit comprises a database with stored reference values for the first target biomarkers and second target biomarkers and a data processor comprising instructions for carrying out a comparison of the relative intensity of each first target biomarker and each second target biomarker to reference values for the first and second target biomarkers and assessing the subject based on the comparison.

In still yet another embodiment, the present disclosure relates to a device for assessing a subject suspected of having or having multisystem inflammatory syndrome in children (MIS-C) or respiratory syncytial virus, where the device comprises:
a. a measuring unit for determining a relative intensity of each of a plurality of first target biomarkers in a sample obtained from a subject suspected of having MIS-C or respiratory syncytial virus, wherein the measuring unit comprises a detection system for detecting and measuring an amount of each of the plurality of first target biomarkers and further wherein the first target biomarkers comprise: Granulocyte-Macrophage Colony-Stimulating Factor (GM-CSF), Pentraxin-3 (PTX-3), marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23); and
b. an evaluation unit operably linked to the measuring unit comprising a database with stored reference values for the first target biomarkers and a data processor comprising instructions for carrying out a comparison of the relative intensity of each first target biomarker to reference values for the target biomarkers and assessing the subject based on the comparison, said evaluation unit being capable of automatically receiving the relative intensity values for each of the biomarkers from the measuring unit.

In one aspect of the device, the first target biomarkers further comprise one or more of perforin (Perf), C-X-C motif chemokine ligand 11 (CXCL11), Epidermal Growth Factor (EGF), interleukin-10 (IL-10), interleukin-8 (IL-8), interleukin-21 (IL-21), interleukin-2 (IL-2), immunoregulatory alpha globulin (IRA), or any combination thereof.

In one aspect of the device:
a. the measuring unit determines a relative intensity of each of a plurality of first target biomarkers and second target biomarkers in the sample, wherein the measuring unit comprises a detection system for detecting and measuring an amount of each of the plurality of first target biomarkers and second target biomarkers and further wherein the second target biomarkers comprise one or more of Granzyme B (GRAND B), fractalkine (CX3CL1), C-C motif chemokine ligand 24 (CCL24), interleukin-7 (IL-7), C-C chemokine ligand 20 (CCL20), interleukin-6 (IL-6), C-C chemokine ligand 17 (CCL17), interleukin-4 (IL-4), interleukin-17 (IL-17), interleukin-15 (IL-15), tumor necrosis factor alpha (TNFα), interleukin-1β (IL-1β), interferon gamma (INFγ), interleukin-12 (IL-12), or any combination thereof; and
b. the evaluation unit comprises a database with stored reference values for the first target biomarkers and second target biomarkers and a data processor comprising instructions for carrying out a comparison of the relative intensity of each first target biomarker and each second target biomarker to reference values for the first and second target biomarkers and assessing the subject based on the comparison.

In some aspects, in any of the above-described devices, the detection system comprises at least one detection agent capable of specifically detecting each of the first target biomarkers, the second target biomarkers, or both the first target biomarkers and the second target biomarkers. Specifically, the detection agent can be an antibody or antibody binding fragment thereof.

These and other aspects and embodiments of the disclosure are described in more detail below.

### BRIEF DESCRIPTION OF THE DRAWINGS

The accompanying drawings are included to provide a further understanding of the methods and compositions of the disclosure and are incorporated in and constitute a part of this specification. The drawings illustrate one or more embodiment(s) of the disclosure, and together with the description serve to explain the principles and operation of the disclosure.

This patent or patent application file contains at least one drawing executed in color. Copies of this patent or patent application publication with color drawings will be provided by the Office upon request and payment of the necessary fee.

FIG. 1 shows GCFP assay and wavelength pathway. Capture antibodies (e.g., detection agents) are immobilized onto a gold-coated nanoscale grating surface chip by pin spotting. Chips are then fitted with plexiglass windows and gaskets to create a flow cell for fluid to pass over the surface of the printed chip. Fluid-containing potential analytes can then be run over the assembled chip, followed by biotinylated secondary antibodies and then streptavidin-Alexafluor647, with washes in between steps with PSB-T. The chip can then be placed in a GCFP reader where a laser illuminates the chip, the fluorescence intensity collected with a camera, and each region of interest (ROI) is analyzed using Enhanced Fluorescence Reader software, V2.3.

FIGS. 2A-D show the development of sandwich-based Enzyme-Linked Immunoassay (ELISA) assay on a gold-coated nanoscale grating surface chip. FIG. 2A shows capture antibodies (e.g., detection agents) for IL-6 and IL-8 were immobilized onto the chip, along with the negative control (PBS) and two positive controls; biotin-BSA (BLC-BSA, to show streptavidin binding) and Alexafluor 647-BSA (to show stabilization during washing), 20 ng/mL of recombinant IL-6 and IL-8 were flowed over the chip. The fluorescence intensity was collected as previously described. Fluorescent intensity within the ROIs for IL-6, IL-8, and positive controls was significantly higher than the negative control. FIG. 2B shows forty-two capture antibodies (e.g., detection agents) were immobilized onto the chip, 20 ng/mL each of recombinant CCL5, galactin-3, and CCL7 diluted in PBS was run over the chip and the fluorescence intensity was collected for each ROI as previously described. The fluorescent intensity within the ROIs for CCL5, galactin-3, and CCL7 were significantly higher than the negative control. FIG. 2C shows forty-two capture antibodies (e.g., detection agents) were immobilized onto the chip, 20 ng/mL of recombinant protein CCL5 and CCL7 diluted in human saliva was run over the chip and the fluorescence intensity was collected for each ROI as previously described. The fluorescent intensities within the ROIs for CCL5 and CCL7 were significantly higher than the negative control. FIG. 2D shows a heat map generated using the detection ratio, which was normalized to the negative control to compare data in B and C. One-way ANOVA analysis was performed (*=p<0.05, **=p<0.01, ***=p<0.001, ****=p<0.0001. The data is presented as the average of five ROIs for each analyte (+/−standard error of the mean).

FIGS. 3A-C show an example of patient (210041005) data generated from GCFP assay. FIG. 3A shows forty-two unique capture antibodies (e.g., detection agents) were immobilized on regions of interest in sets of five ROIs on the chip, patient sample number 210041005 was run over the chip, and an image of the GCFP sensor chip output was captured.

FIG. 3B shows fluorescent intensities within the ROIs were collected as previously described, detecting cystatin C, Marapsin, D-dimer, galaectin-3, and IL-8. FIG. 3C shows a heat map of the detection ratio that was generated and shows detection of the same analytes as in B. One-way ANOVA analysis was performed (*=p<0.05, **=p<0.01, ***=p<0.001, ****=p<0.0001. The data are presented as the average of five ROIs for each analyte (+/−standard error of the mean).

FIGS. 4A-B show the GCFP detection ratio for patient saliva samples. Saliva samples were obtained from each of the patient cohorts; 54 samples (Severe COVID-19 (A1)=15, MIS-C (A2)=9, Mild COVID-19 (A3)=6, Kawasaki Disease (B1)=4, Respiratory Viral Disease (B2)=13, Control (B3)=7) were run over the first-generation biosensor chip. FIG. 4A shows heat maps were generated for each cohort using the mean detection ratio for each analyte, showing candidate biomarkers for the MIS-C(CXCL10), Mild COVID-19 (sDC25), and Kawasaki Disease (IL-10 and IL-2) cohorts. FIG. 4B shows heat maps generated for each patient to demonstrate individual variation in disease state.

FIGS. 5A-B show the GCFP detection ratio for serum samples from patient cohorts. Serum samples from each of the cohorts were tested on the first generation GCFP biosensor chip. Fifty-six samples (Severe COVID-19 (A1)=13, MIS-C (A2)=12, Kawasaki Disease (B1)=12, Respiratory Viral Disease (B2)=12, Control (B3)=7) were tested. FIG. 5A shows heat maps that were generated for each cohort using the mean detection ratio for each analyte, showing a more robust signature per cohort than within saliva. FIG. 5B shows heat maps generated for each patient, showing individual variation in disease state.

FIGS. 6A-I show salivary microbiome of samples analyzed using GCFP. From saliva samples analyzed by GCFP using the first-generation GCFP biosensor chip, 34 samples were also analyzed by 16S RNA high throughput sequencing. Relative abundance at species level was calculated. FIG. 6A shows the relative abundance of the top 20 most abundant taxa across all samples. FIGS. 6B-6I shows plots of the relative abundance for eight bacterial species that were elevated in one or more outlier samples from analyte data. Error bars represent the 95% confidence interval of the median. Points in red were identified as outliers by the robust nonlinear regression (ROUT) method. Samples that were analyte outliers and had an increase in that species are labeled with their cohort.

FIGS. 7A-G show potential analytes found by Microsphere immunoassay (MIA) that can inform the evolving composition of a second-generation GCFP biosensor chip. FIG. 7A shows a heat map generated from microbead immunoassay from serum samples for each cohort. * Indicates a significant difference in that cytokine for the MIS-C (A2) cohort for CXCL11, IL-8, IL-6, IL-4, IL-13, & IL-10 analytes. (FIG. 7B-7G) The antibody response to the SARS-CoV-2 nucleocapsid and spike components (full spike, RBD, S1, S2), the MIS-C cohort had a significantly higher IgA response to SARS-COV-2 spike domains as compared to the COVID-19 cohort. One-way ANOVA analysis was performed (*=p<0.05, **=p<0.01, ***=p<0.001, ****=p<0.0001.

FIGS. 8A-B show a comparison of USA vs Colombia MIS-C patient samples by GCFP microarray. FIG. 8A shows a heat map of MIS-C serum samples comparing the U.S. to Colombia samples. The U.S. patient population has a more robust signature. FIG. 8B shows a heat of MIS-C saliva samples comparing U.S. to Colombia samples, U.S. has a more robust signature.

FIGS. 9A-F show serum from cohorts and individual patient comparison of GCFP microarray assay to microsphere immunoassay (MIA). To compare a traditional MIA with the GCFP microarray, 11 analytes were included in both assays. FIG. 9A shows a heat map generated by log transforming GCFP detection ratios and using the mean of each analyte detection ratio for each cohort. Of the 11 analytes, five were below the detection limit of the GCFP first-generation biosensor chip. FIG. 9B shows a heat map generated by log transforming GCFP detection ratios from individual serum samples from the Severe COVID-19 cohort. FIG. 9C shows a heat map generated by log transforming GCFP detection ratios from individual serum samples from the MIS-C cohort. FIG. 9D shows a heat map generated by log transforming GCFP detection ratios from individual serum samples from the Kawasaki Disease cohort. FIG. 9E shows a heat map generated by log transforming GCFP detection ratios from individual serum samples from the Respiratory Viral Disease cohort. FIG. 9F shows a heat map generated by log transforming GCFP detection ratios from individual serum samples from the Control cohort. Sixteen (16) of the 44 shared serum samples had similar biomarker signatures, while IL-4, IL-6 and CCL20 were not consistently detected on the GCFP chip.

FIGS. 10A-B show the optimization of a first-generation GCFP chip. FIG. 10A shows measurements of the limit of detection for 11 analytes from the first-generation biosensor chip. 1 ng/mL is the limit of detection for most analytes in this assay. FIG. 10B shows optimizing reagents can increase the limit of detection.

FIG. 11 shows the composition of the first-generation chip with 33 analytes and the second-generation chip with 43 analytes. Analytes marked in red were included in Gen 1, and removed from Gen 2. Analytes marked in green were not in Gen 1 and are included in Gen 2. Those analytes marked in orange were originally proposed, but available reagents were not suitable for GCFP.

FIGS. 12A-12E show matched pair ELISA reagent validation results. Forty-two commercially available matched pair antibodies were validated by ELISA using recombinant proteins. Nine commercially available kits that did not meet the necessary ELISA standards when performed following manufacture protocols and were omitted from further analysis.

FIG. 13 shows the testing of a GCFP biosensor chip for the target biomarkers shown in Table A on serum samples from 31 patients with MIS-C and 41 patients with Kawasaki disease.

FIG. 14 shows the testing (group mean) of a GCFP biosensor chip for the target biomarkers shown in Tables A-C on serum samples from 18 patients with severe COVID (A1); 31 patients with MIS-C (A2); 41 patients with Kawasaki Disease (B1); and 15 patients with RSV (B2); there were 28 control patients (B3).

FIG. 15 shows the testing (individual data) of a GCFP biosensor chip for the target biomarkers shown in Tables A-C on serum samples from 18 patients with severe COVID (A1); 31 patients with MIS-C (A2); 41 patients with Kawasaki Disease (B1); 15 patients with RSV (B2); there were 28 control patients (B3).

### DETAILED DESCRIPTION

### I. Definitions

The following terms are used to describe the invention of the present disclosure. In instances where a term is not specifically defined herein, that term is given an art-recognized meaning by those of ordinary skill applying that term in context to its use in describing the present disclosure.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single-letter nucleotides (A, C, G, T, U) and standard single-letter amino acids (A, C, D, E, F, G, H, I, K, L, M, N, P, Q, R, S, T, V, W, or Y) are used herein.

Compounds and materials are described using standard nomenclature. Unless defined otherwise, all technical and scientific terms used herein have the same meaning as is commonly understood by one of skill in the art to which this invention belongs. The following terms are used to describe the invention of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted. The opened ended term “comprising” includes the intermediate and closed terms “consisting essentially of” and “consisting of.” Only the transitional phrases “consisting of’ and “consisting essentially of’ shall be closed or semi-closed transitional phrases, respectively, as set forth in the United States Patent Office Manual of Patent Examining Procedures, Section 2111.03.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. All ranges disclosed herein include both end points as discrete values as well as all integers and fractions specified within the range. For example, a range of 0.1-2.0 includes 0.1, 0.2, 0.3, 0.4 . . . 2.0. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “adenoviruses” refers to medium-sized, non-enveloped viruses with an icosahedral capsid structure containing a linear double-stranded DNA (dsDNA) genome. Adenoviruses belong to the family Adenoviridae and infect a wide range of vertebrates, including humans, mammals, birds, reptiles, amphibians, and fish. Adenoviruses are known for their stability in the environment and resistance to disinfectants due to the absence of a lipid envelope. In humans, adenoviruses are associated with a variety of infections affecting the respiratory tract (e.g., colds, pneumonia), gastrointestinal system (e.g., diarrhea), eyes (e.g., conjunctivitis), and urinary tract. Most infections are mild and self-limiting but can be severe in immunocompromised individuals or young children. These viruses are transmitted through respiratory droplets, direct contact with contaminated surfaces, or fecal-oral routes. Adenoviruses are also widely used as vectors in gene therapy and vaccine development due to their ability to efficiently deliver genetic material into host cells.

As used herein, the term “administering” or “providing” means the actual physical introduction of a composition into or onto (as appropriate) a subject, a host, or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein. “Providing” means giving, administering, selling, distributing, transferring (for profit or not), manufacturing, compounding, or dispensing.

As used herein, the term “amount” refers to the absolute amount of a compound, such as a target biomarker, the relative amount or concentration of a compound (i.e., target biomarker) as well as any value or parameter which correlates thereto or can be derived therefrom. Such values or parameters comprise intensity signal values from all specific physical or chemical properties obtained from said compounds (i.e., one or more target biomarkers) by direct measurements, e.g., intensity values in mass spectra or NMR spectra. Moreover, encompassed are all values or parameters which are obtained by indirect measurements, as described herein, such response levels determined from biological read out systems in response to the compound or intensity signals obtained from specifically bound ligands (e.g., one or more detectable labels). It is understood that values correlating to the aforementioned amounts or parameters can be obtained using standard mathematical operations. Determining an amount of one or more target biomarkers according to the methods described herein can be carried out by any technique which allows for detecting the presence or absence or the amount of a second molecule upon its release from a first molecule. Suitable techniques depend on the molecular nature and the properties of the target biomarkers and are discussed further herein.

Typically, the amount of a target biomarker as referred to in accordance with the present disclosure can be determined by immunoassays using sandwich, competition, or other assay formats. Such assays will develop a signal which is indicative for the presence or absence or the amount of a target biomarker. Further suitable methods comprise measuring a physical or chemical property specific for the target biomarker such as its precise molecular mass or NMR spectrum. Such methods may comprise biosensors, optical devices coupled to immunoassays, biochips, analytical devices such as mass-spectrometers, NMR analyzers, surface plasmon resonance measurement equipment, or chromatography devices. Further, methods include micro-plate ELISA-based methods and fully automated or robotic immunoassays. Suitable measurement methods according the present disclosure may also include precipitation (particularly immunoprecipitation), electrochemiluminescence (electro-generated chemiluminescence), RIA (radioimmunoassay), ELISA (enzyme-linked immunosorbent assay), electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art, such as gel electrophoresis, 2D gel electrophoresis, SDS polyacrylamid gel electrophoresis (SDS-PAGE) or Western Blotting.

As used herein, the term “Angiotensin-Converting Enzyme 2” or “ACE2” as used interchangeably herein refers to an enzyme that plays a crucial role in the renin-angiotensin-aldosterone system (RAAS), which regulates blood pressure. It is primarily found in two forms: membrane-bound ACE2 (mACE2), attached to the surface of cells in tissues such as the intestines, kidneys, heart, and lungs, and soluble ACE2 (sACE2), which is released into the bloodstream after being cleaved from the membrane by the enzyme ADAM17. ACE2 functions as a counterbalance to the angiotensin-converting enzyme (ACE) by converting angiotensin II, a vasoconstrictor, into angiotensin (1-7), a vasodilator, thereby reducing blood pressure. Additionally, ACE2 serves as a receptor for certain coronaviruses, including SARS-CoV and SARS-CoV-2, facilitating their entry into host cells.

As used herein, the term “assessing” refers to assessing whether a subject suffers from multisystem inflammatory syndrome in children, Kawasaki disease, SARS-CoV-2, respiratory syncytial virus, an adenovirus, influenza A, B C, or D, or parainfluenza or is at risk of suffering from multisystem inflammatory syndrome in children, Kawasaki disease, SARS-CoV-2, respiratory syncytial virus, an adenovirus, influenza A, B C, or D, or parainfluenza. Accordingly, assessing as used herein includes diagnosing multisystem inflammatory syndrome in children, Kawasaki disease, SARS-CoV-2, respiratory syncytial virus, an adenovirus, influenza A, B C, or D, or parainfluenza or predicting the risk for developing multisystem inflammatory syndrome in children, Kawasaki disease, SARS-CoV-2, respiratory syncytial virus, an adenovirus, influenza A, B C, or D, or parainfluenza. Typically, the assessment referred to in accordance with the present disclosure is the assessment of the risk of developing or not developing (e.g., predicting the risk of developing or not developing) multisystem inflammatory syndrome in children, Kawasaki disease, SARS-CoV-2, respiratory syncytial virus, an adenovirus, influenza A, B C, or D, or parainfluenza. Alternatively, the assessment referred to in accordance with the present disclosure is the assessment that the subject has or does not have multisystem inflammatory syndrome in children, Kawasaki disease, SARS-CoV-2, respiratory syncytial virus, an adenovirus, influenza A, B C, or D, or parainfluenza.

As used herein, the term “B-lymphocyte activation antigen B7”, “cluster of differentiation 80”, or “B7-1”, as used interchangeably herein refers to a type I membrane protein belonging to the immunoglobulin superfamily. It is primarily expressed on the surface of activated antigen-presenting cells (APCs) such as dendritic cells, macrophages, and activated B cells, as well as on activated T cells. CD80 plays a crucial role in the immune response by serving as a ligand for the T-cell surface receptors CD28 and CTLA-4. The interaction with CD28 provides a costimulatory signal essential for T-cell activation, proliferation, and cytokine production, while binding to CTLA-4 delivers a coinhibitory signal that downregulates T-cell activity to prevent excessive immune responses.

CD80 is composed of two extracellular immunoglobulin-like domains (IgV and IgC) and has a higher affinity for CD28 compared to CD86, another B7 protein. It is involved in various immune processes, including T-cell differentiation and the regulation of both adaptive and innate immune responses. The expression of CD80 is increased by the presence of microbes and cytokines, ensuring that costimulatory signals are available during immune responses.

As used herein, the term “computer-implemented” means that the methods of the present disclosure as described herein in Section III (“Methods of Assessing Subjects Suspected of having Multisystem inflammatory syndrome in children, Kawasaki Disease, SARS-CoV-2, Respiratory Syncytial Virus, an Adenovirus, Influenza A, B, C, or D, and/or Parainfluenza”) are carried out in an automated fashion on a data processing unit which comprises a computer or similar data processing device. The data processing unit receives values for the amount or concentration of each of the target biomarkers (e.g., from any or all of the set of first target biomarkers, second target biomarkers, third target biomarkers and/or fourth target biomarkers). Such values can be the amounts, relative amounts, or any other calculated value reflecting the amount as described in at least Section III (“Methods of Assessing Subjects Suspected of having Multisystem inflammatory syndrome in children, Kawasaki Disease, SARS-CoV-2, Respiratory Syncytial Virus, an Adenovirus, Influenza A, B, C, or D, and/or Parainfluenza”) herein in detail. Accordingly, it is to be understood that when the method is computer-implemented it does not require the determination of the amounts for the target biomarkers but rather uses values for already predetermined amounts.

As used herein, the term “C-reactive protein” or “CRP” as used interchangeably herein, refers to an annular, pentameric protein produced primarily by the liver in response to inflammatory stimuli. It belongs to the pentraxin family and plays a crucial role in the innate immune system as a pattern recognition receptor. CRP levels rapidly increase in response to inflammation, infection, or tissue damage, making it a valuable biomarker for monitoring acute and chronic inflammatory conditions, such as infections, autoimmune diseases, and cardiovascular diseases.

CRP functions by binding to phosphocholine residues on damaged cells and pathogens, which activates the complement system and enhances phagocytosis by macrophages. This process helps clear necrotic and apoptotic cells, as well as bacterial pathogens, from the body. CRP also interacts with Fc receptors, releasing pro-inflammatory cytokines that amplify the immune response.

As used herein, the term “C-C Motif Chemokine Ligand 4 or “CCL4” as used interchangeably herein refers to a small cytokine belonging to the CC chemokine subfamily. It is encoded by the CCL4 gene located on chromosome 17 in humans. CCL4 functions primarily as a chemoattractant, recruiting immune cells such as natural killer cells, monocytes, and T cells to sites of inflammation or tissue damage. It binds to G protein-coupled receptors, particularly CCR5 and CCR8, facilitating its biological effects.

CCL4 is produced by various cell types, including monocytes, B cells, T cells, dendritic cells, neutrophils, fibroblasts, endothelial cells, and epithelial cells, often in response to inflammatory stimuli or antigen presentation. It plays a significant role in immune responses, including inflammation and the modulation of immune cell migration. Additionally, CCL4 is recognized as a major HIV-suppressive factor produced by CD8+ T cells, as it can inhibit HIV entry into host cells by binding to the CCR5 receptor.

As used herein, the term “chemokine ligand 5” or “CCL5” as used interchangeably herein refers to a small cytokine belonging to the CC chemokine subfamily. It is encoded by the CCL5 gene located on chromosome 17q11.2-q12 in humans. CCL5 functions primarily as a chemoattractant, recruiting various immune cells such as T cells, eosinophils, basophils, monocytes, natural killer cells, dendritic cells, and mast cells to sites of inflammation or tissue damage.

Several cell types, including T cells, monocytes, epithelial cells fibroblasts, and thrombocytes produce CCL5. It binds to G protein-coupled receptors, particularly CCR1, CCR3, CCR4, and CCR5, with the highest affinity for CCR5. The interaction between CCL5 and its receptors triggers signaling pathways that lead to cell migration, activation, and proliferation, playing a crucial role in immune responses and inflammation.

As used herein, the term “C-C chemokine ligand 17” or “CCL17,” as used interchangeably herein, refers to a chemokine belonging to the CC chemokine subfamily. It is produced by antigen-presenting cells such as dendritic cells, macrophages, and monocytes, as well as in the thymus. CCL17 functions primarily as a chemoattractant, recruiting immune cells like T-helper cells and regulatory T cells (Tregs) by binding to the CCR4 receptor. CCL17 plays a complex role in various immune processes and diseases. It is involved in recruiting Tregs, which can, depending on the context, either promote immune tolerance or facilitate tumor immune evasion.

As used herein, the term “C-C chemokine ligand 20” or “CCL20” as used interchangeably herein refers to a cytokine belonging to the CC chemokine family. It is encoded by the SCYA20 gene located on chromosome 2 in humans. CCL20 functions primarily as a chemoattractant, recruiting immune cells such as immature dendritic cells, effector/memory T cells, and B cells by binding to its sole receptor, CCR6.

CCL20 is expressed in various tissues, including the liver, lymph nodes, appendix, and epithelial cells, particularly in mucosal surfaces like the intestines and lungs. Its expression is induced by inflammatory stimuli such as tumor necrosis factor (TNF), interleukin-1β (IL-1β), and microbial factors like lipopolysaccharide (LPS). CCL20 plays a crucial role in both innate and adaptive immune responses, facilitating the recruitment of immune cells to sites of inflammation and contributing to the formation and function of mucosal lymphoid tissues.

In addition to its chemotactic properties, CCL20 exhibits antimicrobial activities, similar to those of β-defensins, which also bind to CCR6. This dual functionality makes CCL20 an important component of host defense mechanisms, particularly at mucosal barriers.

As used herein, the term “C-C motif chemokine ligand 24” or “CCL24” refers to a cytokine belonging to the CC chemokine family. It is encoded by the CCL24 gene located on human chromosome 7. CCL24 functions primarily as a chemoattractant, recruiting immune cells such as eosinophils, resting T lymphocytes, and, to a lesser extent, neutrophils by binding to the CCR3 receptor.

CCL24 plays a significant role in allergic diseases and parasitic infections, where it promotes the recruitment of eosinophils to sites of inflammation. It is also involved in proinflammatory and profibrotic processes, contributing to conditions such as systemic sclerosis (SSc), asthma, and idiopathic pulmonary fibrosis (IPF). In these diseases, CCL24 can enhance inflammation and fibrosis by activating fibroblasts and promoting collagen production.

Additionally, CCL24 has been implicated in pregnancy-related processes, where it influences trophoblast function and may affect placental development. Its expression is regulated by various factors, including steroid hormones and decidual stromal cells.

As used herein, the term “C-X-C motif chemokine ligand 10” or “CXCL10”, as used interchangeably herein, refers to a cytokine belonging to the CXC chemokine family. It is encoded by the CXCL10 gene located on human chromosome 4. CXCL10 functions primarily as a chemoattractant, recruiting immune cells such as monocytes, macrophages, T cells, natural killer (NK) cells, and dendritic cells by binding to its receptor, CXCR3.

CXCL10 is secreted in response to interferon-gamma (IFN-γ) by various cell types, including monocytes, endothelial cells, and fibroblasts. It plays a crucial role in inflammatory responses, promoting cell adhesion to endothelial cells and inhibiting angiogenesis, which can have both anti-tumor effects and contribute to tissue damage in conditions like spinal cord injury and autoimmune diseases. CXCL10 is involved in several diseases, including infectious diseases, cancer, autoimmune conditions, and cardiovascular diseases, where it influences disease severity and progression

As used herein, the term “C-X-C motif chemokine ligand 11”, “T cell alpha chemoattractant”, or “CXCL11” as used interchangeably herein, refers to a cytokine belonging to the CXC chemokine family. It is encoded by the CXCL11 gene located on human chromosome 4. CXCL11 functions primarily as a chemoattractant, recruiting immune cells such as activated T cells, natural killer (NK) cells, and monocytes/macrophages by binding to its receptors, CXCR3 and CXCR7. CXCR3 is the primary receptor, with CXCL11 having the highest affinity among its ligands.

CXCL11 is induced by interferons, particularly IFN-γ and IFN-β, and plays a crucial role in inflammatory responses and immune cell migration. It is involved in various physiological and pathological processes, including cancer, where its role can be both anti-tumorigenic by promoting the infiltration of cytotoxic T cells and inhibiting angiogenesis, and pro-tumorigenic by activating oncogenic signaling pathways in certain contexts

As used herein, the term “Cystatin C” or “CysC” as used herein refers to a non-glycosylated, basic protein encoded by the CST3 gene, produced by virtually all nucleated cells at a relatively constant rate. It is a potent inhibitor of lysosomal proteinases and extracellular cysteine proteases, which is crucial in regulating protein degradation, apoptosis, and immune response modulation.

Cystatin C is primarily recognized as a biomarker for renal function, particularly for estimating the glomerular filtration rate (GFR). The kidneys freely filter it and then reabsorbed and catabolized in the proximal tubule, with minimal urinary excretion. Unlike creatinine, cystatin C levels are less influenced by factors such as age, sex, muscle mass, and diet, making it a more accurate and reliable marker for kidney function in diverse populations, including children, the elderly, and those with muscle-wasting conditions.

Beyond its role in assessing kidney function, cystatin C has been linked to cardiovascular disease risk and mortality, often serving as a superior predictor compared to creatinine-based GFR estimates. It is also explored for its potential in monitoring liver cirrhosis, kidney transplant efficacy, and certain oncological conditions. Additionally, cystatin C may play a role in predicting neurological disorders, such as Alzheimer's disease, due to its association with amyloid protein deposition.

As used herein, the term “detection agent” or “biomarker detection agent” as used interchangeably herein refers to any agent which specifically binds to a target biomarker, i.e., an agent which does not cross-react with other components present in the sample. In some embodiments, a detection agent that specifically binds to a target biomarker can be an antibody, an antibody binding fragment or derivative thereof, an aptamer, a ligand for the target biomarker, a receptor for the target biomarker, an enzyme known to bind and/or convert the target biomarker, or a small molecule known to bind to the target biomarker specifically. As used herein, the term “antibody” or “antibodies”, when used as detection agents, includes both polyclonal and monoclonal antibodies, as well as fragments thereof, such as Fv, Fab and F(ab)₂ fragments that are capable of binding antigen or hapten. The present disclosure also includes single-chain antibodies and humanized hybrid antibodies wherein amino acid sequences of a non-human donor antibody exhibiting a desired antigen-specificity are combined with human acceptor antibody sequences. The donor sequences usually include at least the antigen-binding amino acid residues of the donor but may comprise other structurally and/or functionally relevant amino acid residues of the donor antibody as well. Such hybrids can be prepared by methods well-known in the art. Aptamer detection agents, e.g., may be nucleic acid or peptide aptamers. Methods to prepare such aptamers are well-known in the art. For example, random mutations can be introduced into the nucleic acids or peptides being the basis for aptamers. These derivatives can then be tested for binding according to screening procedures known in the art, e.g., phage display. Specific binding of a detection agent means that it should not bind substantially to, i.e., cross-react with, another peptide, polypeptide or substance present in the sample to be analyzed. In some embodiments, the specifically bound target biomarker should be bound with at least 3 times higher, at least 10 times higher, or at least 50 times higher affinity than any other components of the sample. Non-specific binding may be acceptable, if it can still be distinguished and measured unequivocally, e.g., according to its size on a Western Blot, or by its relatively higher abundance in the sample. In some embodiments, the detection agent may be fused or linked permanently or reversibly to a detectable label.

In some embodiments, the disclosure herein contemplates the use of at least two different detection agents for each target biomarker (e.g., a first detection agent and a second detection agent). If more than two different detection agents are to be used for each target biomarker, these would be indicated as a first detection agent, a second detection agent, a third detection agent, a fourth detection agent, or a fifth detection agent.

In these embodiments, a first detection agent, which is also referred to herein as a “capture reagent” or “capture specific binding partner”, specifically binds to at least one epitope on the target biomarker. In some aspects, the first detection agent (e.g., capture reagent or capture specific binding partner), is immobilized on a solid support, such as a particle, microparticle, bead, plate, well, or chip. In some aspects, the chip can be a Grating Coupled Fluorescence Plasmonic (GCFP) microarray biosensor chip. Methods for attaching capture reagents to solid supports are well-known in the art. The second detection agent, which is also referred to herein as a “detection reagent” or “detection specific binding partner”, specifically binds to at least one epitope on the target biomarker which is different than the epitope that the first detection agent specifically binds to. In some aspects, the second detection agent (e.g., detection reagent or detection-specific binding partner) is labeled with one or more detectable labels using routine techniques known in the art.

As used herein, the term “determining” refers to the qualitative and quantitative determination of one or more target biomarkers (e.g., the first target biomarkers, the second target biomarkers, the third target biomarkers and/or the fourth target biomarkers) of the present disclosure. Specifically, the term encompasses the determination of the presence or absence or the absolute or relative amount of the target biomarkers.

As used herein, the term “Epidermal Growth Factor” or “EGF” as used interchangeably herein refers to a protein that plays a crucial role in stimulating cell growth, differentiation, and survival by binding to its receptor, Epidermal Growth Factor Receptor (EGFR). Human EGF is a 53-amino acid polypeptide with a molecular mass of approximately 6 kDa, containing three intramolecular disulfide bonds. It is produced in various tissues, including the kidneys, salivary glands, and digestive tract.

EGF regulates cellular processes such as proliferation, migration, and differentiation in epithelial tissues, fibroblasts, and endothelial cells. It enhances metabolic activity, promotes DNA synthesis, and supports tissue repair, as demonstrated by its role in healing oral and gastroesophageal ulcers. EGF is also implicated in neurogenesis and neuromodulation, influencing the development and function of neurons in the central nervous system. In addition to its physiological roles, EGF has been linked to malignant transformation and cancer progression, with elevated levels observed in certain tumors.

“Epitope,” or “epitopes,” or “epitopes of interest” refer to a site(s) on any molecule, such as a target biomarker that is recognized and can bind to a complementary site(s) on its detection agent, such as, for example a capture and/or detection specific binding partner. The molecule (e.g., target biomarker) and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, a detection agent, such as an antibody or aptamer, as discussed further herein.

As used herein, the term “Ferritin” as used interchangeably herein refers to a universal intracellular and extracellular protein responsible for storing and releasing iron in a controlled manner. It comprises 24 subunits that form a hollow spherical nanocage capable of holding up to 4500 iron atoms, keeping iron in a soluble and non-toxic form. Ferritin plays a crucial role in maintaining iron homeostasis, protecting cells from the oxidative damage caused by free iron through its ferroxidase activity, which converts ferrous (Fe²⁺) to ferric (Fe³⁺) iron.

In clinical medicine, serum ferritin is used as a biomarker to assess total body iron stores, aiding in diagnosing and managing iron deficiency anemia and iron overload conditions like hemochromatosis. Elevated serum ferritin levels can also indicate inflammation, as it is released into the bloodstream when white blood cells are activated. Additionally, ferritin has been linked to various diseases, including cardiovascular conditions, neurodegenerative disorders, and certain cancers, where its levels may serve as a prognostic factor. Ferritin's role extends beyond iron storage; it is involved in cellular redox balance and has novel functions in delivering iron and other molecules, making it a versatile protein with implications in both health and disease.

As used herein, the term “fibrinogen” refers to a soluble plasma glycoprotein, primarily synthesized in the liver, that plays a crucial role in blood coagulation and hemostasis. It is composed of three polypeptide chains: alpha (Aα), beta (Bβ), and gamma (γ), encoded by the FGA, FGB, and FGG genes, respectively, located on chromosome 4 in humans. Fibrinogen circulates in the blood as a hexameric molecule with a molecular weight of approximately 340-420 kDa.

Fibrinogen's primary function is to serve as a precursor to fibrin, which is formed when thrombin cleaves fibrinogen, forming a blood clot that stops bleeding and supports wound healing. Beyond its role in coagulation, fibrinogen interacts with various cells, including platelets, endothelial cells, and immune cells, influencing cell adhesion, migration, and inflammation.

As an acute-phase protein, fibrinogen levels increase in response to inflammation, tissue injury, and certain cancers, making it a biomarker for conditions such as bacterial infections, chronic obstructive pulmonary disease (COPD), and cardiovascular diseases. Elevated fibrinogen levels can also contribute to thrombosis and vascular injury in these conditions. Its role in disease is multifaceted, involving both protective and pathological mechanisms depending on the context.

As used herein, the term “fractalkine” or “CX3CL1,” as used interchangeably herein, refers to a unique chemokine belonging to the CX3C chemokine family, its sole member. It is encoded by the CX3CL1 gene located on human chromosome 16q21. Fractalkine is characterized by its distinctive structure, featuring a chemokine domain atop a long mucin-like stalk, allowing it to exist in membrane-bound and soluble forms. The soluble form is generated through proteolytic cleavage by metalloproteinases.

CX3CL1 functions as both a chemoattractant and an adhesion molecule. It interacts with its sole receptor, CX3CR1, which is expressed on various immune cells, including T cells, monocytes, macrophages, natural killer (NK) cells, and dendritic cells. This interaction facilitates leukocyte migration, adhesion, and retention, playing a crucial role in inflammatory responses and immune cell trafficking. The membrane-bound form of fractalkine promotes strong adhesion of leukocytes to endothelial cells, while the soluble form acts as a potent chemoattractant for these cells.

Fractalkine is involved in various physiological and pathological processes, including neuroprotection, inflammation, and cancer. It has been implicated in neurodegenerative diseases due to its presence in the brain and its role in microglial cell migration. Additionally, fractalkine is associated with several inflammatory and autoimmune conditions, such as atherosclerosis, rheumatoid arthritis, and glomerulonephritis4. Its role in cancer is complex, with evidence suggesting both tumor-suppressive and tumor-promoting effects depending on the context.

As used herein, the term “Granulocyte-Macrophage Colony-Stimulating Factor” or GM-CSF” as used interchangeably herein, refers to a monomeric glycoprotein cytokine that plays a crucial role in the development and function of myeloid cells, including granulocytes (such as neutrophils, eosinophils, and basophils) and monocytes, which mature into macrophages and dendritic cells. GM-CSF is produced by various cell types, including macrophages, T cells, mast cells, endothelial cells, and fibroblasts, often in response to immune stimuli.

GM-CSF functions by binding to its receptor, composed of one a chain and one p chain. This triggers signaling pathways involving Janus kinase (JAK) and signal transducer and activator of transcription (STAT) proteins. This signaling promotes cell survival, differentiation, and activation, enhancing immune responses against infections and inflammation. GM-CSF is also involved in antigen presentation, phagocytosis, and chemotaxis, contributing to both innate and adaptive immunity.

In clinical settings, GM-CSF is used to stimulate the production of white blood cells in patients undergoing chemotherapy or bone marrow transplantation. Additionally, GM-CSF has been implicated in autoimmune diseases and cancer, where it can modulate immune cell activity and influence disease progression. Its role in inflammation and immune regulation makes GM-CSF a potential target for therapeutic interventions in various conditions.

As used herein, the term “Granzyme B” or “GRAND B”, as used interchangeably herein refers to a serine protease primarily found in the granules of natural killer cells (NK cells) and cytotoxic T cells. It plays a crucial role in inducing apoptosis in target cells, often in conjunction with the pore-forming protein perforin. Granzyme B is involved in various biological processes, including immune surveillance, inflammation, and extracellular matrix remodeling. It is implicated in several diseases, including autoimmune conditions, cancer, and inflammatory disorders, due to its ability to induce cell death and modulate immune responses

As used herein, the term “Heat Shock Protein 70” or “HSP70” as used interchangeably herein, refers to a molecular chaperone that plays a crucial role in maintaining cellular homeostasis by facilitating protein folding, preventing protein aggregation, and promoting cell survival under stress conditions. It is highly conserved across species and is induced in response to various forms of cellular stress, including heat shock, oxidative stress, and inflammation. HSP70 is overexpressed in various cancers, including lung, gastric, pancreatic, and squamous cell carcinoma, and is associated with aggressive disease and poor prognosis. It can be used for tumor detection and monitoring treatment outcomes. Elevated serum HSP70 levels are linked to systemic inflammation and oxidative stress, making it a potential biomarker for conditions like chronic obstructive pulmonary disease (COPD), asthma, and rheumatoid arthritis. HSP70 has both protective and deleterious roles in neurodegenerative diseases such as Alzheimer's, Parkinson's, and amyotrophic lateral sclerosis (ALS), depending on the context. Additionally, HSP70 is involved in protecting the heart from ischemialreperfusion injury and may be associated with heart failure prognosis.

As used herein, the term “immunoregulatory alpha globulin” or “IRA”, as used interchangeably herein, refers to a naturally occurring immunosuppressive protein isolated from human serum. It has been identified as a factor that suppresses T cell-dependent immune responses, including T cell-mediated reactions such as skin graft rejection, lymphocyte blastogenesis, and the production of macrophage immobilization factor (MIF). IRA has been shown to inhibit the formation of rosettes between human peripheral lymphocytes and sheep red blood cells (SRBC), as well as the plaque-forming cell response in mice injected with SRBC, which are indicative of T cell activity.

As used herein, “influenza A” refers to a type of virus belonging to the Orthomyxoviridae family that causes respiratory infections in humans and various animals, including birds, pigs, and other mammals. It is an enveloped virus with a segmented, negative-sense, single-stranded RNA genome consisting of eight segments. These segments encode viral proteins, including the surface glycoproteins hemagglutinin (HA) and neuraminidase (NA), which are critical for viral entry into host cells and release of new virions. Influenza A is highly variable due to its ability to undergo antigenic drift (gradual mutations in HA and NA) and antigenic shift (reassortment of gene segments between different viral strains). These mechanisms enable the virus to evade immune responses, leading to seasonal epidemics and occasional pandemics. Influenza A viruses are classified into subtypes based on their HA and NA proteins (e.g., H1N1, H3N2). The virus spreads via respiratory droplets, direct contact with infected individuals, or contaminated surfaces. Symptoms range from mild (fever, cough, sore throat) to severe (pneumonia, acute respiratory distress syndrome), particularly in high-risk groups such as young children, the elderly, and immunocompromised individuals. Influenza A has significant global health impacts due to its potential for widespread outbreaks and severe disease.

As used herein, “influenza B’ refers to a type of virus within the Orthomyxoviridae family that causes respiratory infections exclusively in humans and certain mammals, such as ferrets, pigs, and seals. It is an enveloped virus with a segmented, negative-sense, single-stranded RNA genome consisting of eight segments. These segments encode essential viral proteins, including hemagglutinin (HA) and neuraminidase (NA), which are involved in viral entry and release. Unlike influenza A, influenza B does not infect animals outside of mammals and does not cause pandemics. However, it can lead to seasonal epidemics and significant morbidity, particularly in children, adolescents, the elderly, and individuals with chronic medical conditions. Influenza B evolves more slowly than influenza A due to its limited antigenic drift but still requires annual vaccine updates to address emerging strains. Historically, influenza B has been divided into two lineages: B/Yamagata and B/Victoria. However, the B/Yamagata lineage may have become extinct due to reduced circulation during the COVID-19 pandemic. Symptoms of influenza B include fever, cough, sore throat, muscle aches, fatigue, and respiratory complications such as pneumonia or bronchitis in severe cases.

As used herein, “influenza C” refers to a type of virus within the Orthomyxoviridae family that primarily causes mild respiratory infections in humans and pigs. It is an enveloped virus with a segmented, negative-sense, single-stranded RNA genome consisting of seven segments, fewer than influenza A and B. Unlike these types, influenza C has only one surface glycoprotein, hemagglutinin-esterase fusion (HEF), which combines the functions of hemagglutinin (HA) and neuraminidase (NA). Influenza C infections are generally less common and less severe compared to influenza A and B. The virus does not cause seasonal epidemics or pandemics and is typically associated with sporadic outbreaks of mild respiratory illness resembling the common cold. Symptoms may include fever, cough, runny nose, and sore throat, with rare progression to more serious complications.

As used herein, “influenza D” refers to a type of virus within the Orthomyxoviridae family that primarily infects cattle and, to a lesser extent, other animals such as pigs. It is an enveloped virus with a segmented, negative-sense, single-stranded RNA genome consisting of seven segments. Unlike influenza A, B, and C, influenza D is not known to cause illness in humans, though serological studies suggest potential exposure in individuals who work closely with livestock, such as cattle farmers. Influenza D is primarily associated with respiratory infections in cattle and has been linked to bovine respiratory disease complex (BRDC), a significant health issue in livestock. The virus spreads through nasal secretions and aerosols over short distances or via direct contact.

As used herein, the term “interferon gamma” or “INFγ”, as used interchangeably herein, refers to a dimeric cytokine that plays a crucial role in both innate and adaptive immunity. It is primarily secreted by CD4+T helper 1 (Th1) cells, CD8+ cytotoxic T cells, natural killer (NK) cells, and natural killer T cells (NKT). IFN-γ functions by binding to its heterodimeric receptor, which activates signaling pathways such as the JAK-STAT pathway, leading to the transcription of target genes involved in immune responses.

IFN-γ is essential for host defense against viral, bacterial, and protozoan infections by promoting macrophage activation, antigen presentation, and cell-mediated immunity. It enhances the expression of major histocompatibility complex (MHC) class I and II molecules, facilitating the recognition and elimination of infected cells by cytotoxic T cells. Additionally, IFN-γ has antiviral, antiproliferative, and apoptotic effects, making it a key player in cancer immunotherapy.

Beyond its pro-inflammatory actions, IFN-γ can also exhibit anti-inflammatory properties by promoting the development of regulatory T cells and inhibiting Th17 cell differentiation, which helps regulate immune responses and prevent excessive tissue damage. Its role in immune regulation makes IFN-γ a potential biomarker for monitoring immune responses and a target for therapeutic interventions in various diseases, including autoimmune conditions and cancers.

As used herein, the term interleukin-2 receptor alpha chain, “IL-2Rα”, or “CD25”, as used interchangeably herein refers to a protein that forms part of the high-affinity interleukin-2 (IL-2) receptor complex. It is encoded by the IL2RA gene located on chromosome 10p15.1 in humans. CD25 is a type I transmembrane protein composed of three domains: an extracellular domain that binds IL-2, a transmembrane domain, and a short cytoplasmic domain that lacks intrinsic signaling capabilities but is essential for receptor assembly and function.

CD25 is expressed on various immune cells, including activated T cells, B cells, NK cells, and monocytes. However, it is most prominently expressed on regulatory T cells (Tregs), where it plays a crucial role in their survival and function by facilitating the consumption of IL-2, thereby limiting its availability to effector T cells. This mechanism helps maintain immune tolerance and prevents excessive immune activation. As a biomarker, soluble CD25 (sIL-2Rα) levels are used to monitor immune responses and disease progression in conditions such as autoimmune diseases, cancer, and severe infections like COVID-19.

As used herein, the term “interleukin-1 beta” or “IL-1β” as used interchangeably herein refers to a potent pro-inflammatory cytokine that serves as a biomarker for inflammation and immune responses. It is produced primarily by macrophages and monocytes in response to infection, cellular injury, or antigenic challenges, and the inflammasome complex mediates its release. IL-1β plays a crucial role in inducing fever, inflammation, and immune cell activation, making it a significant factor in various diseases, including cancer, where it can promote tumor growth and metastasis.

As used herein, the term “interleukin-2” or “IL-2” as used interchangeably herein refers to a pleiotropic cytokine that plays a crucial role in the immune system by regulating the activities of white blood cells, particularly T cells. It is a 15.5-16 kDa protein produced primarily by activated CD4+T helper cells and CD8+ cytotoxic T cells, as well as natural killer (NK) cells and dendritic cells. IL-2 functions by binding to its receptor, which consists of three subunits: alpha (CD25), beta (CD122), and gamma (CD132). This interaction promotes the growth, differentiation, and survival of T cells, including effector T cells, memory T cells, and regulatory T cells (Tregs), which are essential for immune tolerance and preventing autoimmune diseases.

IL-2 is involved in various immune processes, including enhancing NK cell cytotoxicity, promoting the differentiation of T cells into Th1 and Th2 subsets, and inhibiting Th17 differentiation. It is also critical for developing T cell immunologic memory, which is vital for enduring cell-mediated immunity. In clinical settings, IL-2 has been used as an immunotherapeutic agent to treat certain cancers, such as metastatic renal cell carcinoma and melanoma, by boosting immune responses against tumors.

As used herein, the term “interleukin-4” or “IL-4,” as used interchangeably herein, refers to a cytokine that plays a pivotal role in the regulation of immune responses, particularly in the differentiation of naive helper T cells (Th0 cells) into Th2 cells. It is produced primarily by mast cells, Th2 cells, eosinophils, and basophils. IL-4 is crucial for humoral immunity, promoting the proliferation and differentiation of B cells into plasma cells and inducing IgE class switching, which is essential for allergic reactions and responses to parasites.

IL-4 acts through two main receptor complexes: Type I, which includes the IL-4Rα subunit and the common γ chain, primarily found on hematopoietic cells; and Type II, which includes IL-4Rα and IL-13Rα1, found on both hematopoietic and non-hematopoietic cells. This dual receptor system allows IL-4 to influence a wide range of cell types, including macrophages, where it promotes alternative activation into M2 macrophages, which are involved in tissue repair and anti-inflammatory responses. IL-4 is associated with allergic inflammation, asthma, and autoimmune diseases due to its role in Th2 responses. It also plays a role in neurological conditions, such as multiple sclerosis, where it may have protective effects by modulating microglial responses.

As used herein, the term “interleukin-6” or “IL-6,” as used interchangeably herein, refers to a pleiotropic cytokine that plays a crucial role in both innate and adaptive immunity. It is produced by a wide range of cell types, including macrophages, T cells, B cells, fibroblasts, keratinocytes, mesangial cells, vascular endothelial cells, mast cells, and dendritic cells. IL-6 functions as both a pro-inflammatory cytokine and an anti-inflammatory myokine, depending on the context.

IL-6 is involved in various biological processes, including the acute phase response, where it stimulates the production of acute phase proteins such as C-reactive protein (CRP) and fibrinogen. It also promotes fever and supports the growth and differentiation of B cells into antibody-producing plasma cells. Additionally, IL-6 influences T-cell differentiation, favoring Th2 and Th17 responses while inhibiting Th1 polarization.

IL-6 signals through a cell-surface receptor complex consisting of the IL-6Rα chain and the signal-transducing component gp130. This signaling pathway activates the JAK/STAT cascade, leading to the transcription of numerous genes involved in immune responses and inflammation. IL-6 can also signal through a trans-signaling pathway involving soluble IL-6R, allowing it to affect cells that do not express the membrane-bound receptor.

As used herein, the term “interleukin-7” or “IL-7,” as used interchangeably herein, refers to a cytokine that plays a crucial role in the development, survival, and homeostasis of immune cells, particularly T cells, B cells, and natural killer (NK) cells. It is produced by various cell types, including stromal cells in the bone marrow and thymus, keratinocytes, and epithelial cells. IL-7 functions by binding to its receptor, a heterodimer composed of the IL-7Rα chain (CD127) and the common γ-chain, which activates signaling pathways such as JAK/STAT, PI3K/Akt, and MAPK.

IL-7 is essential for the differentiation of hematopoietic stem cells into lymphoid progenitor cells and supports the proliferation and survival of T cells and B cells during their development. It is critical for T cell maturation in the thymus and for maintaining peripheral T cell homeostasis, including the survival of both naive and memory T cells Additionally, IL-7 influences the rearrangement of immunoglobulin and T-cell receptor genes, facilitating the generation of diverse immune repertoires.

As used herein, the term “interleukin-8” or “IL-8” as used interchangeably herein refer to a pro-inflammatory chemokine that plays a crucial role in the recruitment and activation of neutrophils and other immune cells to sites of inflammation. It is produced by a variety of cell types, including macrophages, epithelial cells, endothelial cells, fibroblasts, and tumor cells, often in response to inflammatory stimuli such as cytokines (e.g., IL-1, IL-6, TNF-α), bacterial components, and oxidative stress.

IL-8 functions primarily by binding to its receptors, CXCR1 and CXCR2, which are G protein-coupled receptors. This interaction triggers signaling pathways that induce chemotaxis, promoting the migration of neutrophils and other granulocytes to areas of inflammation. Additionally, IL-8 stimulates phagocytosis and the oxidative burst, enhancing the ability of neutrophils to eliminate pathogens.

As used herein, the term “interleukin-10” or “IL-10” as used interchangeably herein refers to a pleiotropic cytokine that plays a crucial role in modulating inflammation and maintaining immune homeostasis. It is primarily known as an anti-inflammatory cytokine, produced by various cell types, including macrophages, dendritic cells, T cells (especially regulatory T cells), and B cells, in response to immune stimuli. IL-10 functions by inhibiting the production of pro-inflammatory cytokines such as TNF-α, IL-1β, IL-12, and IFN-γ, and it suppresses the antigen-presenting capacity of macrophages and dendritic cells.

Despite its anti-inflammatory properties, IL-10 can also exhibit immunostimulatory effects under certain conditions. It promotes the survival, proliferation, and differentiation of B cells into antibody-producing plasma cells and supports the function of regulatory T cells (Tregs) and regulatory B cells (Bregs). Additionally, IL-10 enhances the cytolytic activity of NK cells and can stimulate mast cells, influencing allergic responses.

IL-10 signals through a tetrameric receptor complex, activating the JAK/STAT pathway, specifically STAT3, which mediates its anti-inflammatory and immunoregulatory effects.

As used herein, the term “interleukin-12” or “IL-12” as used interchangeably herein refers to a heterodimeric cytokine composed of two covalently linked subunits: p35 (IL-12A) and p40 (IL-12B), forming the biologically active IL-12 p70. It is primarily produced by dendritic cells, macrophages, neutrophils, and B cells in response to antigenic stimulation. IL-12 plays a pivotal role in bridging innate and adaptive immunity by promoting the differentiation of naive T cells into Th1 cells, which are crucial for cell-mediated immunity against intracellular pathogens and tumors.

IL-12 functions by inducing the production of interferon-gamma (IFN-γ) from T cells and natural killer (NK) cells, enhancing their cytotoxic activity and promoting the transcription of genes involved in cell-mediated immunity, such as perforin and granzymes. Additionally, IL-12 exhibits anti-angiogenic effects by inducing IFN-γ, which reduces vascular endothelial growth factor production and inhibits endothelial cell adhesion and survival.

IL-12 signaling is mediated through its receptor, composed of IL-12Rβ1 and IL-12Rβ2, activating the JAK/STAT pathway, particularly STAT4, which is essential for its biological effects

As used herein, the terms, “interleukin-13” or “IL-13” as used interchangeably herein refers to a cytokine primarily produced by T helper 2 (Th2) cells, natural killer T cells, mast cells, basophils, eosinophils, and nuocytes. It plays a central role in allergic inflammation, particularly in conditions like asthma and atopic dermatitis. IL-13 is involved in the regulation of IgE synthesis, goblet cell hyperplasia, mucus hypersecretion, and airway hyperresponsiveness. It shares many biological activities with IL-4, as both cytokines use the IL-4 receptor alpha chain for signaling, but IL-13 does not induce Th2 cell differentiation like IL-4 does.

IL-13 functions by binding to its receptor, which includes the IL-13Rα1 and IL-4Rα subunits, activating the STAT6 pathway. This signaling promotes the transcription of genes involved in allergic responses, such as those encoding mucins and eotaxins. Despite its pro-inflammatory effects in allergic diseases, IL-13 also exhibits anti-inflammatory properties by inhibiting the production of pro-inflammatory cytokines.

As used herein, the term “interleukin-15” or “IL-15” as used interchangeably herein refers to a pleiotropic cytokine that plays a crucial role in the development, maintenance, and function of immune cells, particularly natural killer (NK) cells, CD8+ T cells, and memory T cells. It is a member of the four α-helix bundle family of cytokines, similar to IL-2, and shares some receptor subunits with IL-2, including the IL-2/IL-15 receptor beta chain (CD122) and the common gamma chain (γc, CD132). However, IL-15 also binds to a unique IL-15 receptor alpha chain (IL-15Rα), which provides specificity to its signaling pathway.

IL-15 is produced by a variety of cell types, including monocytes, macrophages, dendritic cells, keratinocytes, and fibroblasts. It is involved in both innate and adaptive immunity, promoting the proliferation and survival of immune cells, and preventing apoptosis by inducing anti-apoptotic proteins like Bcl-2 and Bcl-xL. IL-15 is essential for the development and maintenance of NK cells and memory T cells, supporting their homeostatic proliferation and function.

As used herein, the term “interleukin-17” or “IL-17” as used interchangeably herein refers to a pro-inflammatory cytokine produced by T helper 17 (Th17) cells, as well as other immune cells like γδ T cells, natural killer T (NKT) cells, and Type 3 innate lymphoid cells (ILC3). It plays a crucial role in linking T cell activation to neutrophil mobilization and activation, thereby mediating protective innate immunity against pathogens and contributing to the pathogenesis of inflammatory diseases.

IL-17 functions by inducing the production of various inflammatory molecules, including chemokines (e.g., CXCL1, CXCL2, CXCL8), granulopoiesis factors (e.g., G-CSF), and pro-inflammatory cytokines (e.g., IL-6, TNF-α, IL-1β). It promotes the recruitment of neutrophils to sites of inflammation and supports the expression of antimicrobial peptides and acute phase proteins, which are essential for host defense against extracellular pathogens, such as bacteria and fungi.

As used herein, the term “interleukin-21” or “IL-21” as used interchangeably herein refers to a type I cytokine that plays a pivotal role in regulating immune responses, particularly in the coordination of innate and adaptive immunity. It is primarily produced by activated CD4+ T cells, including T follicular helper (Tfh) cells and Th17 cells, as well as natural killer T (NKT) cells and Hodgkin's lymphoma cells.

IL-21 functions by binding to its receptor, IL-21R, which is expressed on a wide range of immune cells, including B cells, T cells, NK cells, dendritic cells, and macrophages. This interaction activates the JAK/STAT signaling pathway, particularly STAT3, which mediates IL-21's biological effects.

IL-21 has diverse roles in the immune system, including: (1) Enhancing B cell proliferation and differentiation into plasma cells, which is crucial for antibody production and class switching; (2) Promoting the expansion and cytotoxic activity of CD8+ T cells, supporting antiviral and antitumor responses; (3) Supporting the differentiation of Th17 cells, which are involved in defense against extracellular pathogens; and (4) Regulating NK cell activity, enhancing their cytotoxicity and survival.

As used herein, the term “interleukin-23” or “IL-23,” as used interchangeably herein, refers to a heterodimeric cytokine composed of two subunits: the p40 subunit, which is shared with IL-12, and a unique p19 subunit. It is primarily produced by activated macrophages and dendritic cells in response to antigenic stimulation, particularly in tissues such as the skin, intestinal mucosa, and lungs.

IL-23 plays a crucial role in bridging innate and adaptive immunity by promoting the expansion and survival of Th17 cells, which are a subset of T helper cells that produce IL-17A and other pro-inflammatory cytokines. It also activates other immune cells, including γδ T cells, natural killer T cells (NKTs), mucosal-associated invariant T cells (MAITs), and IL-17-secreting innate lymphoid cells (ILC3s), collectively known as type 3 immune cells or type-17 cells.

IL-23 functions by binding to its receptor, which consists of the IL-23R and IL-12Rβ1 subunits, activating signaling pathways involving Janus kinases (JAKs), such as JAK2 and TYK2, and signal transducers and activators of transcription (STATs), particularly STAT3 and STAT4. This signaling cascade promotes the expression of genes involved in inflammation and immune responses, including those encoding RORγt, a transcription factor essential for Th17 cell differentiation.

As used herein, the term “interleukin-27” or “IL-27” as used interchangeably herein refers to a heterodimeric cytokine composed of two subunits: Epstein-Barr virus-induced gene 3 (EBI3) and IL-27p28. It is primarily produced by activated antigen-presenting cells (APCs), such as dendritic cells and macrophages, in response to immune stimuli. IL-27 binds to its receptor, which consists of IL-27Rα (WSX-1) and gp130, activating signaling pathways including the JAK/STAT and p38 MAPK pathways.

IL-27 plays a complex role in immune regulation, exhibiting both pro-inflammatory and anti-inflammatory effects depending on the context. It promotes the differentiation of Th1 cells by inducing the expression of T-bet and IFN-γ, which are crucial for cell-mediated immunity against intracellular pathogens. Conversely, IL-27 suppresses the differentiation of Th2 and Th17 cells by inhibiting the transcription factors GATA-3 and RORγt, respectively, thereby reducing the production of pro-inflammatory cytokines like IL-17 and IL-5.

IL-27 also induces the production of IL-10, an anti-inflammatory cytokine, and enhances the expression of co-inhibitory receptors such as PD-L1, which help limit excessive immune responses and prevent tissue damage.

As used herein, the term “interleukin-33” or “IL-33” as used interchangeably herein refers to a cytokine belonging to the IL-1 superfamily, encoded by the IL33 gene. It is constitutively expressed in structural and lining cells, including fibroblasts, endothelial cells, and epithelial cells, particularly in tissues exposed to the environment, such as the skin, gastrointestinal tract, and lungs. IL-33 lacks a secretory signal peptide and is primarily stored in the nucleus, acting as a transcriptional repressor. It is released in its full-length form (amino acids 1-270) during cell necrosis or tissue damage, functioning as an alarmin to alert the immune system to potential threats.

IL-33 signals through the ST2 receptor (IL1RL1), which is expressed on various immune cells, including Th2 cells, group 2 innate lymphoid cells (ILC2s), mast cells, eosinophils, basophils, and dendritic cells. This interaction activates signaling pathways involving NF-κB, JNK, and p38 MAPK, promoting the production of Th2-associated cytokines like IL-4 and IL-5, which are crucial for type 2 immune responses.

As used herein the term “Kawasaki disease” refers to an acute condition that primarily affects pediatric subjects under the age of five, though it can also occur in older pediatric subjects and human adult subjects. It is characterized by inflammation of the blood vessels throughout the body, particularly affecting the coronary arteries that supply blood to the heart. This inflammation can lead to serious complications if not treated promptly. While the exact cause of Kawasaki's disease is unknown, it is believed to be linked to genetic predisposition or triggered by infections. Symptoms of Kawasaki's disease include a high fever lasting more than five days, accompanied by at least four of the following symptoms: rash, swollen lymph glands in the neck, red eyes, red, dry, cracked lips and a “strawberry tongue”, swollen hands and feet, which may peel at a later time, or any combinations thereof. Untreated Kawasaki disease can lead to heart problems, such as coronary artery aneurysms, which are bulges in the artery walls that can rupture or lead to blood clots.

As used herein, the term “label” or “detectable label,” as used interchangeably herein, refers to a moiety that is attached, fused, or linked permanently or reversibly to a detection agent (such as a detection reagent or a detection specific binding partner) to render the reaction between the detection agent (such as a capture reagent or capture specific binding partner) and the target biomarker detectable. Suitable detectable labels are any labels that are detectable by any appropriate detection method. Examples of labels include gold particles, latex beads, acridan ester, luminol, ruthenium, enzymatically active labels, radioactive labels, magnetic labels (e.g., “magnetic beads”, including paramagnetic and superparamagnetic labels), and fluorescent labels. Enzymatically active labels include, for example, horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star™ (Amersham Biosciences), ECF™ (Amersham Biosciences). A suitable enzyme-substrate combination may result in a colored reaction product, fluorescence or chemiluminescence, which can be measured according to methods known in the art (e.g., using a light-sensitive film or a suitable camera system). As for measuring the enyzmatic reaction, the criteria given above apply analogously. Typical fluorescent labels include fluorescent proteins (such as GFP and its derivatives), Cy3, Cy5, Texas Red, Fluorescein, and the Alexa dyes (e.g., Alexa 568). Further fluorescent labels are available, for example, from Molecular Probes (Oregon). Additionally, the use of quantum dots as fluorescent labels is also contemplated herein. Typical radioactive labels include 35S, 1251, 32P, 33P and the like. A radioactive label can be detected by any method known and appropriate, e.g. a light-sensitive film or a phosphor imager. Suitable labels may also be or comprise tags, such as biotin, digoxygenin, His-Tag, Glutathion-S-Transferase, FLAG, GFP, myc-tag, influenza A virus haemagglutinin (HA), maltose binding protein, and the like.

As used herein, the term “Marapsin”, “Mar” or “pancreasin” as used interchangeably herein refers to a trypsin-like serine protease. It is a member of a large protease gene cluster located on human chromosome 16p13.3, which includes other structurally related proteases like testisin, tryptase epsilon, tryptase gamma, and EOS. Marapsin is primarily expressed in pancreatic tissue but has also been found in stratified squamous epithelia, such as the esophagus, tonsil, cervix, and larynx. Its expression is notably upregulated in the hyperproliferative epidermis of psoriatic lesions and regenerating skin wounds, suggesting a role in re-epithelialization and keratinocyte differentiation.

As used herein, the term “multisystem inflammatory syndrome in children” or “MIS-C” as used interchangeably herein refers to a condition that occurs in subjects, specifically, pediatric subjects, following exposure to SARS-CoV-2, the virus responsible for COVID-19. It is characterized by a severe inflammatory response affecting multiple organ systems, including the heart, blood vessels, gastrointestinal organs, lungs, kidneys, skin, eyes, and nervous system. The syndrome is non-specific and is associated with one or more of: abdominal pain, diarrhea, vomiting, rashes, red eyes, red or swollen hands/feet, red cracked lips, cough, sore throat, fever, cardiovascular dysfunction, and respiratory dysfunction. MIS-C can lead to severe complications such as shock, organ failure, and cardiac dysfunction, necessitating intensive care and prompt medical intervention. Despite its severity, most pediatric subjects recover with appropriate treatment

As used herein, “optional” or “optionally” means that the subsequently described event or circumstance may or may not occur, and that the description includes instances where said event or circumstance occurs and instances where it does not.

As used herein, “parainfluenza” refers to a group of viruses known as human parainfluenza viruses (HPIVs), which are single-stranded, enveloped RNA viruses belonging to the Paramyxoviridae family. These viruses are a common cause of respiratory infections in humans, particularly in children, infants, older adults, and individuals with weakened immune systems. There are four major types of HPIVs (HPIV-1, HPIV-2, HPIV-3, and HPIV-4), each associated with distinct respiratory illnesses. HPIVs can cause both upper respiratory tract infections (e.g., colds, sore throat) and lower respiratory tract infections (e.g., croup, bronchiolitis, pneumonia). Symptoms vary depending on the infection site and may include runny nose, cough, fever, wheezing, hoarseness, and difficulty breathing. HPIV-1 is most commonly linked to croup outbreaks, while HPIV-3 is associated with bronchiolitis and pneumonia. These viruses are transmitted through respiratory droplets or contact with contaminated surfaces and are highly contagious. While infections are often mild in healthy individuals, they can lead to severe complications in vulnerable populations. Reinfections are common throughout life but tend to be less severe after the first exposure.

As used herein, a “pediatric subject” refers to a subject less than 18 years of age (i.e., not 18 years of age or older). For example, a pediatric subject may be less than about 18 years old, or about 17 years old, about 16 years old, about 15 years old, about 14 years old, about 13 years old, about 12 years old, about 11 years old, about 10 years old, about 9 years old, about 8 years old, about 7 years old, about 6 years old, about 5 years old, about 4 years old, about 3 years old, about 2 years old, about 1 year old, or less than about 1 year old. In some aspects, the pediatric subject may be less than about 1 year old to about less than 18 years old. In some aspect, the pediatric subject may be less than about 1 year old to about 17 years old. For example, a pediatric subject may be anywhere from about one day, about two days, about three days, about four days, about five days, about six days, about one week, about two weeks, about three weeks, about one month, about two months, about three months, about four months, about five months, about six months, about seven months, about eight months, about nine months, about ten months, or about eleven months, in total, less than: about 18 years old, or about 17 years old, or about 16 years old, or about 15 years old, or about 14 years old, or about 13 years old, or about 12 years old, or about 11 years old, or about 10 years old, or about 9 years old, or about 8 years old, or about 7 years old, or about 6 years old, or about 5 years old, or about 4 years old, or about 3 years old, or about 2 years old, or about 1 year old, or less than about 1 year old.

As used herein, the term “Pentraxin-3” or “PTX-3,” as used interchangeably herein, refers to a pentraxin protein that plays a crucial role in humoral innate immunity. It is produced locally by various cell types, including myeloid cells, endothelial cells, and respiratory epithelial cells, in response to pro-inflammatory signals, microbial recognition, and tissue damage. Unlike C-reactive protein (CRP), which is primarily produced by the liver, PTX3 is synthesized at the site of inflammation, allowing for rapid responses to pathogens and tissue injury.

PTX3 functions as a pattern recognition molecule, binding to various pathogens, including fungi, bacteria, and viruses, facilitating their opsonization and clearance by phagocytic cells. Depending on the context, it modulates complement activation, neutrophil recruitment, and inflammation, acting as a pro-inflammatory and anti-inflammatory mediator. Additionally, PTX3 is involved in tissue remodeling and repair, influencing fibrin deposition and fibrinolysis.

As used herein, the term “Perforin” or “Per” as used interchangeably herein refers to a glycoprotein that plays a crucial role in the immune system by forming pores in the membranes of target cells, leading to cell lysis and death. It is primarily produced by cytotoxic T lymphocytes (CTLs) and natural killer (NK) cells, which are key components of cell-mediated immunity. Perforin acts by polymerizing into a cylindrical structure that inserts into the target cell membrane, creating pores that allow the entry of granzymes, a family of serine proteases that induce apoptosis (programmed cell death) in the target cells.

Perforin's mechanism of action involves binding to the target cell membrane through interactions with phospholipids facilitated by calcium ions. The pores formed by perforin enable the diffusion of granzymes into the cytosol, where they can activate caspases and initiate the apoptotic cascade. This process is essential for eliminating infected cells and tumor cells, thus playing a critical role in immune surveillance and defense against infections.

As used herein, the term “reference value(s)” refers to an amount, score, or level that allows for the allocation of a subject into a group of subjects that are either suffering from a disease or condition or at risk of developing a disease or condition from a group of subjects that do not suffer from the same disease or condition or which are not at risk of developing the same disease or condition. Such a reference value(s) can be a threshold amount, score, or level which separates these groups from each other. Thus, a reference value is an amount, score, or level that allows for allocation of a subject into a group of subjects suffering from a disease or condition or being at risk for developing it or not. In some aspects, a reference value can be an amount, score, or level for a single target biomarker which allows for the allocation of a subject into: (1) a group of subjects being at risk of developing or having multisystem inflammatory syndrome or not being at risk of developing or not having multisystem inflammatory syndrome; (2) a group of subjects being at risk of developing or having Kawasaki disease or not being at risk of developing or not having Kawasaki disease; (3) a group of subjects being at risk of developing or having Severe Acute Respiratory Syndrome Coronavirus 2 (SARS-CoV-2); (4) a group of subjects being at risk of developing or having respiratory syncytial virus; (5) a group of subjects being at risk of developing or having an adenovirus; (6) a group of subjects being at risk of developing or having influenza A, B C, or D; and/or (7) a group of subjects being at risk of developing or having parainfluenza. In other aspects, a reference value can be an amount, score, or level for a combination of target biomarkers which allows for the allocation of a subject into: (1) a group of subjects being at risk of developing or having multisystem inflammatory syndrome or not being at risk of developing or not having multisystem inflammatory syndrome; (2) a group of subjects being at risk of developing or having Kawasaki disease or not being at risk of developing or not having Kawasaki disease; (3) a group of subjects being at risk of developing or having Severe Acute Respiratory Syndrome Coronavirus 2 (SARS-CoV-2); (4) a group of subjects being at risk of developing or having respiratory syncytial virus; (5) a group of subjects being at risk of developing or having an adenovirus; (6) a group of subjects being at risk of developing or having influenza A, B C, or D; and/or (7) a group of subjects being at risk of developing or having parainfluenza.

A “reference value range” refers to range of amounts, scores, or levels that allows for the allocation of a subject into a group of subjects that are either suffering from a disease or condition or at risk of developing a disease or condition from a group of subjects that do not suffer from the same disease or condition or which are not at risk of developing the same disease or condition. Such a reference value(s) can be a threshold amount, score, or level which separates these groups from each other. Thus, a reference value range is a range of amounts, scores, or levels that allow for allocation of a subject into a group of subjects suffering from a disease or condition or being at risk for developing it or not. In some aspects, a reference value range can be a range of amounts, scores, or levels for a single target biomarker which allows for the allocation of a subject into: (1) a group of subjects being at risk of developing or having multisystem inflammatory syndrome or not being at risk of developing or not having multisystem inflammatory syndrome; (2) a group of subjects being at risk of developing or having Kawasaki disease or not being at risk of developing or not having Kawasaki disease; (3) a group of subjects being at risk of developing or having Severe Acute Respiratory Syndrome Coronavirus 2 (SARS-CoV-2); (4) a group of subjects being at risk of developing or having respiratory syncytial virus; (5) a group of subjects being at risk of developing or having an adenovirus; (6) a group of subjects being at risk of developing or having influenza A, B C, or D; and/or (7) a group of subjects being at risk of developing or having parainfluenza. In other aspects, a reference value range can be a range of amounts, scores, or levels for a combination of target biomarkers which allows for the allocation of a subject into: (1) a group of subjects being at risk of developing or having multisystem inflammatory syndrome or not being at risk of developing or not having multisystem inflammatory syndrome; (2) a group of subjects being at risk of developing or having Kawasaki disease or not being at risk of developing or not having Kawasaki disease; (3) a group of subjects being at risk of developing or having severe Acute Respiratory Syndrome Coronavirus 2 (SARS-CoV-2); (4) a group of subjects being at risk of developing or having respiratory syncytial virus; (5) a group of subjects being at risk of developing or having an adenovirus; (6) a group of subjects being at risk of developing or having influenza A, B C, or D; and/or (7) a group of subjects being at risk of developing or having parainfluenza.

A suitable reference value or reference value range separating two groups can be calculated using statistical methods known in the art, such as those referred to herein, based on amounts of target biomarkers from either a subject or group of subjects known to suffer from a disease or condition or being at risk for developing it or a subject or group of subjects known not to suffer from a disease or condition or being at risk for developing it. The reference values for an individual subject may vary depending on various physiological parameters such as age, gender, or subpopulations.

In some aspects, reference values or reference value ranges can be calculated for a cohort of subjects based on the average or mean values for a given parameter such as biomarker amount by applying standard statistically methods. In particular, the accuracy of a test, such as a method aiming to diagnose a disease or condition or not, is best described by its receiver-operating characteristics (ROC). A ROC graph plots all of the sensitivity/specificity pairs resulting from continuously varying the decision threshold over the entire range of data observed. The clinical performance of a diagnostic method depends on its accuracy, i.e., its ability to allocate subjects to a certain prognosis or diagnosis correctly. The ROC plot indicates the overlap between the two distributions by plotting the sensitivity versus 1-specificity for the complete range of thresholds suitable for making a distinction. On the y-axis is sensitivity, or the true-positive fraction, which is defined as the ratio of a number of true-positive test results to the product of a number of true-positive and number of false-negative test results. This has also been referred to as positivity in the presence of a disease or condition. It is calculated solely from the affected subgroup. On the x-axis is the false-positive fraction, or 1-specificity, which is defined as the ratio of a number of false-positive results to the product of a number of true-negative and the number of false-positive results. It is an index of specificity and is calculated entirely from the unaffected subgroup. Because the true- and false-positive fractions are calculated entirely separately by using the test results from two different subgroups, the ROC plot is independent of the prevalence of the event in the cohort. Each point on the ROC plot represents a sensitivity/-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 450 diagonal line from the lower left corner to the upper right corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the test's overall accuracy. Depending on a desired confidence interval, a threshold can be derived from the ROC curve, allowing for the diagnosis or prediction of a given event with a proper balance of sensitivity and specificity, respectively. Accordingly, the reference value or reference value ranges to be used for the aforementioned method of the present invention, i.e., a threshold amount or range of threshold amounts that allow the discrimination between subjects being at risk and not being at risk, can be generated, usually by establishing an ROC for said cohort as described above and deriving a threshold amount or threshold amounts therefrom. The ROC plot allows deriving suitable thresholds depending on the desired sensitivity and specificity for a diagnostic method. It will be understood that an optimal sensitivity is desired for excluding a subject for being at increased risk or for suffering from a disease or condition (i.e., a rule out) whereas an optimal specificity is envisaged for including/assessing a subject for being at an increased risk or for suffering from the disease or condition (i.e. a rule in).

As used herein, “relative intensity” as used in connection with a biomarker (e.g., a target biomarker), such as to determine the “relative intensity of a target biomarker”, refers to determining the proportion or level of a specific biomarker (e.g., target biomarker) compared to other biomarkers or a reference value. Relative intensity is a method used to assess the presence and strength of a biomarker in a sample. Methods for determining relative intensity are well known in the art.

For example, determining the relative intensity of a biomarker in a Grating-Coupled Fluorescence Plasmonic (GCFP) chip involves fluorescence signal acquisition, normalization, and comparative analysis using specialized hardware and software. Generally, capture reagents or capture specific binding partners (e.g., capture antibodies) specific to target biomarkers are printed onto a gold-coated nanoscale grating surface, creating distinct regions of interest (ROIs) for each analyte. ROIs are validated using recombinant proteins to confirm specificity and limit of detection. The biological sample is recirculated over the chip, allowing the target biomarkers to bind to their cognate capture reagents (e.g., capture antibodies). Detection reagents or detection specific binding partners (e.g., detection antibodies), labeled with a detectable label, such as biotinylated secondary antibodies and streptavidin-AlexaFluor 647, are used to generate fluorescence signals proportional to the target biomarker concentration. A GCFP reader measures fluorescence intensity at each ROI using surface plasmon resonance-enhanced fluorescence. Signals are collected as raw fluorescence values (e.g., arbitrary units from the detector). Fluorescence intensities from negative control ROIs are subtracted to eliminate nonspecific background signals. Relative intensity is calculated as a ratio of the fluorescence signal at each biomarker-specific ROI to the background signal. In some aspects, the detection ratio is calculated using the below formula:

$$Detection Ratio = \frac{Fluorescence Intensity ( Biomarker ROI )}{Fluorescence Intensity ( Negative Control ROI )}$$

This normalization accounts for chip-to-chip variability and matrix effects (e.g., serum sample versus saliva sample). Normalized detection ratios are visualized in heat maps to compare biomarker intensities across patient cohorts (e.g., MIS-C vs. Kawasaki disease, MIS-C vs. COVID-19, and/or MIS-C vs. RSV). Fluorescence signals are adjusted to stay within a linear dynamic range (e.g., 1-2000 arbitrary units) to ensure quantitative accuracy. Results are cross-validated against established assays (e.g., Luminex microsphere immunoassays) to confirm correlation and refine biomarker panels for subsequent chip generations.

As used herein “Respiratory Syncytial Virus” or “RSV” as used interchangeably herein refers to a highly contagious virus that primarily infects the respiratory tract, including the nose, throat, lungs, and breathing passages. It is a negative-sense, single-stranded RNA virus belonging to the Pneumoviridae family and the genus Orthopneumovirus. The virus derives its name from the formation of large, multinucleated cells called syncytia when infected cells fuse. RSV is a leading cause of respiratory infections across all age groups but poses the greatest risk to infants, young children, older adults, and individuals with weakened immune systems. It is one of the most common causes of bronchiolitis and pneumonia in children under one year of age and can cause severe lower respiratory tract infections in high-risk populations. The virus spreads through respiratory droplets from coughing or sneezing, direct contact with an infected person, or contaminated surfaces. Symptoms range from mild, cold-like signs (e.g., runny nose, cough) to severe respiratory distress requiring hospitalization in vulnerable individuals.

As used herein, the term “sample” or “biological sample” as used interchangeably herein refers to any sample obtained from a subject that under physiological conditions comprises any or all of the target biomarkers described herein (e.g., any or all of the first target biomarkers, the second target biomarkers, the third target biomarkers, and/or the fourth target biomarkers). In some embodiments, the sample may comprise a fluid sample or a tissue sample. In some embodiments, the sample is a blood sample (e.g., such as a capillary, venous or arterial blood sample) or a blood product such as serum or plasma. In some embodiments, the sample comprises urine. In embodiments, the sample is a respiratory specimen, including a nasal sample, an oropharyngeal sample, a mid-turbinate sample, sputum, endotracheal aspirate or bronchoalveolar lavage. In some embodiments, the sample is a cerebrospinal fluid sample. In some embodiments, the sample is a saliva sample. In some embodiments, the sample is a tissue sample. In some embodiments, the sample is obtained from a subject suspected of having multisystem inflammatory syndrome in children (MIS-C). In some embodiments, the sample is obtained from a subject suspected of having Kawaski Disease. In some embodiments, the sample is obtained from a subject suspected of having a SARS-CoV-2 infection. In some embodiments, the sample is obtained from a subject suspected of having respiratory syncytial virus. In some embodiments, the sample is obtained from a subject suspected of having an adenovirus. In some embodiments, the sample is obtained from a subject suspected of having influenza A, B, C, or D. In some embodiments, the sample is obtained from a subjected suspected of having parainfluenza. In some embodiments, the subject is a human. In other embodiments, the subject is a human pediatric subject. In yet other embodiments, the subject is a human adult subject. The sample can be used directly as obtained from a patient or can be pre-treated, such as by heating, filtration, distillation, extraction, concentration, centrifugation, inactivation of interfering components, addition of reagents, and the like.

As used herein, the term “Serum Amyloid P Component” or “SAP,” as used interchangeably herein, refers to a 25 kDa pentameric glycoprotein belonging to the pentraxin family, which plays a role in the innate immune system. It is synthesized primarily by hepatocytes and circulates in plasma at relatively stable concentrations (10-50 mg/L), unlike other acute-phase proteins like C-reactive protein (CRP), which fluctuates significantly during inflammation. SAP is known for its ability to bind to various molecules through calcium-dependent interactions, including amyloid fibrils, chromatin, and microbial components.

As used herein, the term “sensitivity” of an assay or method as used herein refers to the proportion of subjects for whom the outcome is positive that are correctly identified as positive (e.g., correctly identifying those subjects with a disease or medical condition for which they are being tested). For example, this might include correctly identifying subjects as at risk of having or do have multisystem inflammatory syndrome in children, Kawasaki disease, SARS-CoV-2, respiratory syncytial virus, an adenovirus, influenza A, B C, or D, and/or parainfluenza as distinct from those who are not at risk of having or do not have multisystem inflammatory syndrome in children, Kawasaki's disease, SARS-CoV-2, respiratory syncytial virus, an adenovirus, influenza A, B C, or D, and/or parainfluenza.

As used herein, the term “Severe Acute Respiratory Syndrome Coronavirus 2”, “SARS-CoV-2”, or COVID-19, as used interchangeably herein refers to a highly transmissible and pathogenic virus belonging to the Coronaviridae family and the Betacoronavirus genus. It is a positive-sense, single-stranded RNA virus which emerged in late 2019 and led to a global pandemic. SARS-CoV-2 is zoonotic in origin, with genetic evidence suggesting it likely originated from bat coronaviruses, potentially through an intermediate host. It infects human cells by binding to the angiotensin-converting enzyme 2 (ACE2) receptor, primarily targeting epithelial cells in the respiratory tract. This interaction facilitates viral replication and spread, which can result in a wide range of clinical manifestations, from asymptomatic cases to severe respiratory failure and multi-organ damage. The virus's genome is approximately 30,000 bases long, making it one of the largest among RNA viruses. A notable feature of SARS-CoV-2 is its furin cleavage site on the spike protein, which enhances its ability to infect human cells and contributes to its high transmissibility compared to other coronaviruses.

As used herein, the term “specificity” of an assay or method as used herein refers to the proportion of subjects for whom the outcome is negative that are correctly identified as negative (e.g., correctly identifying those subjects who do not have a disease or medical condition for which they are being tested). For example, this might include correctly identifying subjects not at risk or not having multisystem inflammatory syndrome in children, Kawasaki disease, SARS-CoV-2, respiratory syncytial virus, an adenovirus, influenza A, B C, or D, and/or parainfluenza as distinct from those who are at risk or do have multisystem inflammatory syndrome in children, Kawasaki disease, SARS-CoV-2, respiratory syncytial virus, an adenovirus, influenza A, B C, or D, and/or parainfluenza.

As used herein, the term “specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

As used herein, the term “subject” and “patient” as used interchangeably refers herein to refer to an animal, such as a mammal, including a primate (such as a human, a non-human primate, e.g., a monkey, and a chimpanzee), a non-primate (such as a cow, cattle, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject or patient is a human subject or a human patient, such as a human being treated or assessed for a disease, disorder or condition, a human at risk for a disease, disorder or condition, a human having a disease, disorder or condition, and/or human being treated for a disease, disorder or condition as described herein. In some aspects, the subject is a human subject. In yet other aspects, the subject is a pediatric subject, e.g., a human pediatric subject. In still further aspects, the subject is an adult subject, e.g., a human adult subject. As used herein, a “human adult subject” is a human subject at 18 years of age or older. In some embodiments, the human adult subject is about 18-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above-recited ranges are also intended to be part of this invention. In addition, ranges of values using a combination of any of the above-recited values as upper and/or lower limits are intended to be included. As used herein, a subject is “in need of treatment” if such subject would benefit biologically, medically, or in quality of life from such treatment. A subject in need of treatment does not necessarily present symptoms, particular in the case of preventative or prophylaxis treatments. As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms also encompass therapy and cure. Treatment means any way the symptoms of a pathological condition, disorder, or disease are ameliorated or otherwise beneficially altered. Preferably, the subject in need of such treatment is a mammal, preferably a human.

As used herein, the term “substantially” means to a great or significant extent, but not completely.

As used herein, the term “Thioredoxin-1” or “Trx-1” refers a multifunctional protein that plays a crucial role in maintaining redox homeostasis within cells. It is a small, ubiquitous protein found in all living cells, primarily localized in the cytosol but also present in the nucleus and extracellularly. Trx-1 contains two redox-active cysteine residues (Cys32 and Cys35) in its active site, which allow it to reduce oxidized proteins by donating electrons, thereby maintaining the thiol groups of these proteins in a reduced state.

Trx-1 is involved in various cellular processes, including cell proliferation, DNA synthesis, modulation of transcription factors, and regulation of cell death. It acts as an antioxidant by scavenging reactive oxygen species (ROS) and restoring the function of oxidized proteins. Additionally, Trx-1 can modulate immune responses by influencing the activity of immune cells and cytokines, such as inhibiting the production of pro-inflammatory cytokines in allergic reactions. Trx-1 is secreted by several cell types, including lymphocytes, hepatocytes, fibroblasts, and tumor cells, and its plasma levels can increase under stress conditions like infections, oxidative stress, and inflammation.

As used herein, the term “Triggering Receptor Expressed on Myeloid Cells,” “TREM,” or “TREM-1,” as used interchangeably herein, refers to a transmembrane receptor primarily expressed on myeloid cells, including neutrophils, monocytes, and some tissue macrophages. It plays a crucial role in amplifying the immune response to microbial products, thereby enhancing inflammation during infections. TREM is also released in a soluble form, known as sTREM or sTREM-1, which serves as a biomarker for disease severity and outcome, particularly in conditions like septic shock. TREM is upregulated in response to bacterial lipopolysaccharide (LPS) and other microbial products, contributing to the inflammatory response by synergizing with Toll-like receptors (TLRs). sTREM is released through proteolytic cleavage, sTREM is a reliable biomarker for TREM pathway activation and disease severity.

As used herein, the term “tumor necrosis factor alpha” or “TNFα” as used interchangeably herein, refers to a pleiotropic cytokine belonging to the TNF superfamily. It is primarily produced by macrophages, monocytes, T cells, and natural killer (NK) cells in response to inflammatory stimuli, such as bacterial products and other cytokines. TNF-α functions by binding to its receptors, TNFR1 and TNFR2, which are part of the TNF receptor superfamily. This interaction triggers signaling pathways leading to various cellular responses, including inflammation, cell survival, proliferation, and apoptosis.

TNF-α plays a crucial role in innate immunity, mediating acute and chronic inflammation. It is involved in the regulation of fever, coagulation, and the production of other cytokines and chemokines. TNF-α is also implicated in autoimmune diseases, such as rheumatoid arthritis and psoriasis, where its dysregulation contributes to chronic inflammation and tissue damage. In cancer, TNF-α can have both tumor-suppressive and tumor-promoting effects, depending on the context.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

A significant change is any detectable change that is statistically significant in a standard parametric or non-parametric test of statistical significance such as Student's t-test or Mann-Whitney U test, where p<0.05.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

### II. Biomarker Panels

In one embodiment, the present disclosure provides biomarker panels or panels, as used interchangeably herein, containing a plurality of detection agents that specifically bind to corresponding target biomarkers. Specifically, the biomarker panels of the present disclosure can be used for assessing whether a subject is suspected of having or has multisystem inflammatory syndrome in children, Kawasaki disease, SARS-CoV-2, respiratory syncytial virus, an adenovirus, influenza A, B, C, or D, or parainfluenza. In some aspects, the subject is a mammal. In some aspects, the subject is a human pediatric subject. In some aspects, the subject is a human adult subject. In other aspects, the subject is a dog. In still other aspects, the subject is a cat. In still other aspects, the subject is a cow or cattle. In still other aspects, the subject is a bird. In still further aspects, the subject is a pig. In some aspects, the biomarker panels are used to determine or predict whether a subject (e.g., such as a pediatric subject) is at risk of developing or not developing multisystem inflammatory syndrome in children, Kawasaki disease, SARS-CoV-2, respiratory syncytial virus, an adenovirus, influenza A, B, C, or D, or parainfluenza. In yet other aspects, the biomarker panels are used to determine or diagnose that a subject (e.g., such as a pediatric subject) has or does not have multisystem inflammatory syndrome in children, Kawasaki disease, SARS-CoV-2, respiratory syncytial virus, an adenovirus, influenza A, B, C, or D, or parainfluenza.

### MIS-C and Kawasaki Disease Biomarker Panels

In one embodiment, the biomarker panel is a panel for assessing a subject suspected of having or having multisystem inflammatory syndrome in children (MIS-C) or Kawasaki disease. The biomarker panel comprises a plurality of first biomarker detection agents that specifically bind to a corresponding set of first target biomarkers. The set of first target biomarkers includes Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), and immunoregulatory alpha globulin (IRA). Thus, the plurality of first detection agents in the biomarker panel includes at least 15 first detection agents. Specifically, the biomarker panel includes at least one first detection agent that specifically binds to PTX-3, at least one first detection agent that specifically binds to IL-10, at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-4, at least one first detection agent that specifically binds to IL-17, at least one first detection agent that specifically binds to CCL5, at least one first detection agent that specifically binds to TNFα, at least one first detection agent that specifically binds to IL-13, at least one first detection agent that specifically binds to INFγ, at least one first detection agent that specifically binds to IL-12, and at least one first detection agent that specifically binds to IRA. Any suitable detection agent known in the art can be used. In some aspects, the detection agent is an antibody or antigen-binding fragment thereof. In another aspect, the detection agent is an aptamer.

In some aspects, one or more of the at least one first detection agent that specifically binds to PTX-3, at least one first detection agent that specifically binds to IL-10, at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-4, at least one first detection agent that specifically binds to IL-17, at least one first detection agent that specifically binds to CCL5, at least one first detection agent that specifically binds to TNFα, at least one first detection agent that specifically binds to IL-13, at least one first detection agent that specifically binds to INFγ, at least one first detection agent that specifically binds to IL-12, at least one first detection agent that specifically binds to IRA or any combination thereof is used or functions as a capture reagent in the panel. In some aspects, when at least one first detection agent that specifically binds to its corresponding target biomarker is used as a capture reagent or capture-specific binding partner, the capture reagent can optionally be immobilized on a solid support. The type of solid support is not critical and can be particle, microparticle, bead, plate, well, chip, or any combination thereof.

In some aspects, one or more of the at least one first detection agent that specifically binds to PTX-3, at least one first detection agent that specifically binds to IL-10, at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-4, at least one first detection agent that specifically binds to IL-17, at least one first detection agent that specifically binds to CCL5, at least one first detection agent that specifically binds to TNFα, at least one first detection agent that specifically binds to IL-13, at least one first detection agent that specifically binds to INFγ, at least one first detection agent that specifically binds to IL-12, at least one first detection agent that specifically binds to IRA, or any combination thereof is labeled with one or more detectable labels and used or functions as a detection reagent or detection specific binding partner in the panel.

In some embodiments, the set of first target biomarkers can include up to 7 additional target biomarkers. Specifically, the set of first target biomarkers can include: Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), immunoregulatory alpha globulin (IRA) and optionally, one or more of the following additional 7 target biomarkers: B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof. In some aspects, all 7 of these additional target biomarkers are included in the set of first target biomarkers, or just 1, 2, 3, 4, 5, or 6 of these additional target biomarkers are included.

For example, in some aspects, the biomarker panel can include at least one first detection agent that specifically binds to PTX-3, at least one first detection agent that specifically binds to IL-10, at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-4, at least one first detection agent that specifically binds to IL-17, at least one first detection agent that specifically binds to CCL5, at least one first detection agent that specifically binds to TNFα, at least one first detection agent that specifically binds to IL-13, at least one first detection agent that specifically binds to INFγ, at least one first detection agent that specifically binds to IL-12, and at least one first detection agent that specifically binds to IRA and, in addition, at least one first detection agent that specifically binds to B7-1, at least one first detection agent that specifically binds to CXCL11, at least one first detection agent that specifically binds to IL-8, at least one first detection agent that specifically binds to IL-7, at least one first detection agent that specifically binds to IL-23, at least one first detection agent that specifically binds to IL-2, at least one first detection agent that specifically binds to IL-15, or any combination thereof. Any suitable detection agent known in the art can be used in the biomarker panel. In some aspects, the detection agent is an antibody or antigen-binding fragment thereof. In another aspect, the detection agent is an aptamer. Additionally, at least one first detection agent can be used or function as a capture reagent or capture specific binding partner that is optionally immobilized on a solid support or as a detection reagent or detection specific binding partner and labeled with a detectable label.

In some aspects, the biomarker panel can include at least one first detection agent that specifically binds to PTX-3, at least one first detection agent that specifically binds to IL-10, at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-4, at least one first detection agent that specifically binds to IL-17, at least one first detection agent that specifically binds to CCL5, at least one first detection agent that specifically binds to TNFα, at least one first detection agent that specifically binds to IL-13, at least one first detection agent that specifically binds to INFγ, at least one first detection agent that specifically binds to IL-12, at least one first detection agent that specifically binds to IRA, at least one first detection agent that specifically binds to B7-1, at least one first detection agent that specifically binds to CXCL11, at least one first detection agent that specifically binds to IL-8, at least one first detection agent that specifically binds to IL-7, at least one first detection agent that specifically binds to IL-23, at least one first detection agent that specifically binds to IL-2, and at least one first detection agent that specifically binds to IL-15. In this aspect, the plurality of first detection agents in the biomarker panel will comprise at least 22 first detection agents.

In some embodiments, in addition to the first set of target biomarkers, the biomarker panel can further comprise a plurality of first biomarker detection agents that specifically bind to a corresponding set of second target biomarkers. The set of second target biomarkers includes one or more of marapsin, fractalkine (CX3CL1), C-C chemokine ligand 20 (CCL20), interleukin-21, C-C chemokine ligand 17 (CCL17), interleukin-1β (IL-1β), or any combinations thereof. In some aspects, all 6 of these additional target biomarkers are included in the set of second target biomarkers, or just 1, 2, 3, 4, or 5 of these second target biomarkers are included.

Thus, in some aspects, the biomarker panel can include:
A. at least one first detection agent that specifically binds to PTX-3, at least one first detection agent that specifically binds to IL-10, at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-4, at least one first detection agent that specifically binds to IL-17, at least one first detection agent that specifically binds to CCL5, at least one first detection agent that specifically binds to TNFα, at least one first detection agent that specifically binds to IL-13, at least one first detection agent that specifically binds to INFγ, at least one first detection agent that specifically binds to IL-12, and at least one first detection agent that specifically binds to IRA; and
B. at least one first detection agent that specifically binds to B7-1, at least one first detection agent that specifically binds to CXCL11, at least one first detection agent that specifically binds to IL-8, at least one first detection agent that specifically binds to IL-7, at least one first detection agent that specifically binds to IL-23, at least one first detection agent that specifically binds to IL-2, at least one first detection agent that specifically binds to IL-15, or any combination thereof. Any suitable detection agent known in the art can be used in the biomarker panel. In some aspects, the detection agent is an antibody or antigen-binding fragment thereof. In another aspect, the detection agent is an aptamer. Additionally, the at least one first detection agent can be used or function as a capture reagent or capture specific binding partner that is optionally immobilized on a solid support or as a detection reagent or detection specific binding partner and labeled with a detectable label.

In yet other aspects, the biomarker panel can include:
A. at least one first detection agent that specifically binds to PTX-3, at least one first detection agent that specifically binds to IL-10, at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-4, at least one first detection agent that specifically binds to IL-17, at least one first detection agent that specifically binds to CCL5, at least one first detection agent that specifically binds to TNFα, at least one first detection agent that specifically binds to IL-13, at least one first detection agent that specifically binds to INFγ, at least one first detection agent that specifically binds to IL-12, and at least one first detection agent that specifically binds to IRA; and
B. at least one first detection agent that specifically binds to B7-1, at least one first detection agent that specifically binds to CXCL11, at least one first detection agent that specifically binds to IL-8, at least one first detection agent that specifically binds to IL-7, at least one first detection agent that specifically binds to IL-23, at least one first detection agent that specifically binds to IL-2 and at least one first detection agent that specifically binds to IL-15. Any suitable detection agent known in the art can be used in the biomarker panel. In some aspects, the detection agent is an antibody or antigen-binding fragment thereof. In another aspect, the detection agent is an aptamer. Additionally, the at least one first detection agent can be used or function as a capture reagent or capture specific binding partner that is optionally immobilized on a solid support or as a detection reagent or detection specific binding partner and labeled with a detectable label.

In still other aspects, the biomarker panel can include:
A. at least one first detection agent that specifically binds to PTX-3, at least one first detection agent that specifically binds to IL-10, at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-4, at least one first detection agent that specifically binds to IL-17, at least one first detection agent that specifically binds to CCL5, at least one first detection agent that specifically binds to TNFα, at least one first detection agent that specifically binds to IL-13, at least one first detection agent that specifically binds to INFγ, at least one first detection agent that specifically binds to IL-12, and at least one first detection agent that specifically binds to IRA; and
B. at least one first detection agent that specifically binds to marapsin, at least one first detection agent that specifically binds to CX3CL1, at least one first detection agent that specifically binds to CCL20, at least one first detection agent that specifically binds to IL-21, at least one first detection agent that specifically binds to CCL17, at least one first detection agent that specifically binds to IL-1β, or any combination thereof. Any suitable detection agent known in the art can be used in the biomarker panel. In some aspects, the detection agent is an antibody or antigen-binding fragment thereof. In another aspect, the detection agent is an aptamer. Additionally, the at least one first detection agent can be used or function as a capture reagent or capture specific binding partner that is optionally immobilized on a solid support or as a detection reagent or detection specific binding partner and labeled with a detectable label.

In still other aspects, the biomarker panel can include:
A. at least one first detection agent that specifically binds to PTX-3, at least one first detection agent that specifically binds to IL-10, at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-4, at least one first detection agent that specifically binds to IL-17, at least one first detection agent that specifically binds to CCL5, at least one first detection agent that specifically binds to TNFα, at least one first detection agent that specifically binds to IL-13, at least one first detection agent that specifically binds to INFγ, at least one first detection agent that specifically binds to IL-12, and at least one first detection agent that specifically binds to IRA; and
B. at least one first detection agent that specifically binds to marapsin, at least one first detection agent that specifically binds to CX3CL1, at least one first detection agent that specifically binds to CCL20, at least one first detection agent that specifically binds to IL-21, at least one first detection agent that specifically binds to CCL17, and at least one first detection agent that specifically binds to IL-1P. Any suitable detection agent known in the art can be used in the biomarker panel. In some aspects, the detection agent is an antibody or antigen-binding fragment thereof. In another aspect, the detection agent is an aptamer. Additionally, the at least one first detection agent can be used or function as a capture reagent or capture specific binding partner that is optionally immobilized on a solid support or as a detection reagent or detection specific binding partner and labeled with a detectable label.

In yet still other aspects, the biomarker panel can include:
A. at least one first detection agent that specifically binds to PTX-3, at least one first detection agent that specifically binds to IL-10, at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-4, at least one first detection agent that specifically binds to IL-17, at least one first detection agent that specifically binds to CCL5, at least one first detection agent that specifically binds to TNFα, at least one first detection agent that specifically binds to IL-13, at least one first detection agent that specifically binds to INFγ, at least one first detection agent that specifically binds to IL-12, and at least one first detection agent that specifically binds to IRA;
B. at least one first detection agent that specifically binds to B7-1, at least one first detection agent that specifically binds to CXCL11, at least one first detection agent that specifically binds to IL-8, at least one first detection agent that specifically binds to IL-7, at least one first detection agent that specifically binds to IL-23, at least one first detection agent that specifically binds to IL-2, at least one first detection agent that specifically binds to IL-15, or any combination thereof; and
C. at least one first detection agent that specifically binds to marapsin, at least one first detection agent that specifically binds to CX3CL1, at least one first detection agent that specifically binds to CCL20, at least one first detection agent that specifically binds to IL-21, at least one first detection agent that specifically binds to CCL17, at least one first detection agent that specifically binds to IL-1β, or any combination thereof. Any suitable detection agent known in the art can be used in the biomarker panel. In some aspects, the detection agent is an antibody or antigen-binding fragment thereof. In another aspect, the detection agent is an aptamer. Additionally, the at least one first detection agent can be used or function as a capture reagent or capture specific binding partner that is optionally immobilized on a solid support or as a detection reagent or detection specific binding partner and labeled with a detectable label.

### MIS-C and Severe Acute Respiratory Syndrome Coronavirus 2 (SARS-CoV-2) Biomarker Panels

In one embodiment, the biomarker panel is a panel for assessing a subject suspected of having or having multisystem inflammatory syndrome in children (MIS-C) or Severe Acute Respiratory Syndrome Coronavirus 2 (SARS-CoV-2). The biomarker panel comprises a plurality of first biomarker detection agents that specifically bind to a corresponding set of first target biomarkers. The set of first target biomarkers includes Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), marapsin, interleukin-2 receptor alpha chain (CD25), Heat Shock Protein 70 (HSP70), and interleukin-23 (IL-23). Thus, the plurality of first detection agents in the biomarker panel includes at least 12 first detection agents. Specifically, the biomarker panel includes at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-17, at least one first detection agent that specifically binds to INFγ, at least one first detection agent that specifically binds to IRA, at least one first detection agent that specifically binds to marapsin, at least one first detection agent that specifically binds to CD25, at least one first detection agent that specifically binds to HSP20, and at least one first detection agent that specifically binds to IL-23. Any suitable detection agent known in the art can be used. In some aspects, the detection agent is an antibody or antigen-binding fragment thereof. In another aspect, the detection agent is an aptamer.

In some aspects, one or more of the at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-17, at least one first detection agent that specifically binds to INFγ, at least one first detection agent that specifically binds to IRA, at least one first detection agent that specifically binds to marapsin, at least one first detection agent that specifically binds to CD25, at least one first detection agent that specifically binds to HSP20, at least one first detection agent that specifically binds to IL-23 or any combination thereof is used or functions as a capture reagent in the panel. In some aspects, when at least one first detection agent that specifically binds to its corresponding target biomarker is used as a capture reagent or capture-specific binding partner, the capture reagent can optionally be immobilized on a solid support. The type of solid support is not critical and can be particle, microparticle, bead, plate, well, chip, or any combination thereof.

In some aspects, one or more of the at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-17, at least one first detection agent that specifically binds to INFγ, at least one first detection agent that specifically binds to IRA, at least one first detection agent that specifically binds to marapsin, at least one first detection agent that specifically binds to CD25, at least one first detection agent that specifically binds to HSP20, at least one first detection agent that specifically binds to IL-23, or any combination thereof is labeled with one or more detectable labels and used or functions as a detection reagent or detection specific binding partner in the panel.

In some embodiments, the set of first target biomarkers can include up to 8 additional target biomarkers. Specifically, the set of first target biomarkers can include: Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), interleukin-23 (IL-23) and optionally, one or more of the following additional 8 target biomarkers: Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7), or any combination thereof. In some aspects, all 8 of these additional target biomarkers are included in the set of first target biomarkers, or just 1, 2, 3, 4, 5, 6, or 7 of these additional target biomarkers are included.

For example, in some aspects, the biomarker panel can include at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-17, at least one first detection agent that specifically binds to INFγ, at least one first detection agent that specifically binds to IRA, at least one first detection agent that specifically binds to Marapsin, at least one first detection agent that specifically binds to CD25, at least one first detection agent that specifically binds to HSP20, and at least one first detection agent that specifically binds to IL-23 and, in addition, at least one first detection agent that specifically binds to IL-4, at least one first detection agent that specifically binds to CCL5, at least one first detection agent that specifically binds to IL-12, at least one first detection agent that specifically binds to CCL20, at least one first detection agent that specifically binds to IL-21, at least one first detection agent that specifically binds to B7-1, at least one first detection agent that specifically binds to IL-8, at least one first detection agent that specifically binds to IL-7, or any combination thereof. Any suitable detection agent known in the art can be used in the biomarker panel. In some aspects, the detection agent is an antibody or antigen-binding fragment thereof. In another aspect, the detection agent is an aptamer. Additionally, at least one first detection agent can be used or function as a capture reagent or capture specific binding partner that is optionally immobilized on a solid support or as a detection reagent or detection specific binding partner and labeled with a detectable label.

In some aspects, the biomarker panel can include at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-17, at least one first detection agent that specifically binds to INFγ, at least one first detection agent that specifically binds to IRA, at least one first detection agent that specifically binds to Marapsin, at least one first detection agent that specifically binds to CD25, at least one first detection agent that specifically binds to HSP20, at least one first detection agent that specifically binds to IL-23, at least one first detection agent that specifically binds to IL-4, at least one first detection agent that specifically binds to CCL5, at least one first detection agent that specifically binds to IL-12, at least one first detection agent that specifically binds to CCL20, at least one first detection agent that specifically binds to IL-21, at least one first detection agent that specifically binds to B7-1, at least one first detection agent that specifically binds to IL-8, and at least one first detection agent that specifically binds to IL-7. In this aspect, the plurality of first detection agents in the biomarker panel will comprise at least 20 first detection agents.

In some embodiments, in addition to the first set of target biomarkers, the biomarker panel can further comprise a plurality of first biomarker detection agents that specifically bind to a corresponding set of second target biomarkers. The set of second target biomarkers includes one or more of pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2), or any combinations thereof. In some aspects, all 12 of these additional target biomarkers are included in the set of second target biomarkers, or just 1, 2, 3, 4, 5, 6, 7, 8, 9, 10, or 11 of these second target biomarkers are included.

Thus, in some aspects, the biomarker panel can include:
A. at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-17, at least one first detection agent that specifically binds to INFγ, at least one first detection agent that specifically binds to IRA, at least one first detection agent that specifically binds to marapsin, at least one first detection agent that specifically binds to CD25, at least one first detection agent that specifically binds to HSP20, and at least one first detection agent that specifically binds to IL-23; and
B. at least one first detection agent that specifically binds to IL-4, at least one first detection agent that specifically binds to CCL5, at least one first detection agent that specifically binds to IL-12, at least one first detection agent that specifically binds to CCL20, at least one first detection agent that specifically binds to IL-21, at least one first detection agent that specifically binds to B7-1, at least one first detection agent that specifically binds to IL-8, at least one first detection agent that specifically binds to IL-7, or any combination thereof. Any suitable detection agent known in the art can be used in the biomarker panel. In some aspects, the detection agent is an antibody or antigen-binding fragment thereof. In another aspect, the detection agent is an aptamer. Additionally, the at least one first detection agent can be used or function as a capture reagent or capture specific binding partner that is optionally immobilized on a solid support or as a detection reagent or detection specific binding partner and labeled with a detectable label.

In some embodiments, in addition to the first set of target biomarkers, the biomarker panel can further comprise a plurality of first biomarker detection agents that specifically bind to a corresponding set of second target biomarkers. The set of second target biomarkers includes one or more of pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2), or any combinations thereof. In some aspects, all 12 of these additional target biomarkers are included in the set of second target biomarkers, or just 1, 2, 3, 4, 5, 6, 7, 8, 9, 10, or 11 of these second target biomarkers are included.

In yet other aspects, the biomarker panel can include:
A. at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-17, at least one first detection agent that specifically binds to INFγ, at least one first detection agent that specifically binds to IRA, at least one first detection agent that specifically binds to marapsin, at least one first detection agent that specifically binds to CD25, at least one first detection agent that specifically binds to HSP20, and at least one first detection agent that specifically binds to IL-23, and
B. at least one first detection agent that specifically binds to IL-4, at least one first detection agent that specifically binds to CCL5, at least one first detection agent that specifically binds to IL-12, at least one first detection agent that specifically binds to CCL20, at least one first detection agent that specifically binds to IL-21, at least one first detection agent that specifically binds to B7-1, at least one first detection agent that specifically binds to IL-8, at least one first detection agent that specifically binds to IL-7, or any combination thereof. Any suitable detection agent known in the art can be used in the biomarker panel. In some aspects, the detection agent is an antibody or antigen-binding fragment thereof. In another aspect, the detection agent is an aptamer. Additionally, the at least one first detection agent can be used or function as a capture reagent or capture specific binding partner that is optionally immobilized on a solid support or as a detection reagent or detection specific binding partner and labeled with a detectable label.

In still yet other aspects, the biomarker panel can include:
A. at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-17, at least one first detection agent that specifically binds to INFγ, at least one first detection agent that specifically binds to IRA, at least one first detection agent that specifically binds to marapsin, at least one first detection agent that specifically binds to CD25, at least one first detection agent that specifically binds to HSP20, and at least one first detection agent that specifically binds to IL-23, and
B. at least one first detection agent that specifically binds to IL-4, at least one first detection agent that specifically binds to CCL5, at least one first detection agent that specifically binds to IL-12, at least one first detection agent that specifically binds to CCL20, at least one first detection agent that specifically binds to IL-21, at least one first detection agent that specifically binds to B7-1, at least one first detection agent that specifically binds to IL-8, and at least one first detection agent that specifically binds to IL-7. Any suitable detection agent known in the art can be used in the biomarker panel. In some aspects, the detection agent is an antibody or antigen-binding fragment thereof. In another aspect, the detection agent is an aptamer. Additionally, the at least one first detection agent can be used or function as a capture reagent or capture specific binding partner that is optionally immobilized on a solid support or as a detection reagent or detection specific binding partner and labeled with a detectable label.

In yet still other aspects, the biomarker panel can include:
A. at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-17, at least one first detection agent that specifically binds to INFγ, at least one first detection agent that specifically binds to IRA, at least one first detection agent that specifically binds to marapsin, at least one first detection agent that specifically binds to CD25, at least one first detection agent that specifically binds to HSP20, and at least one first detection agent that specifically binds to IL-23; and
B. at least one first detection agent that specifically binds to PTX-3, at least one first detection agent that specifically binds to IL-10, at least one first detection agent that specifically binds to TNFα, at least one first detection agent that specifically binds to IL-13, at least one first detection agent that specifically binds to CX3CL1, at least one first detection agent that specifically binds to CCL17, at least one first detection agent that specifically binds to CCL17, at least one first detection agent that specifically binds to IL-1β, at least one first detection agent that specifically binds to perforin, at least one first detection agent that specifically binds to CCL4, at least one first detection agent that specifically binds to GRAND B, at least one first detection agent that specifically binds to CXCL11, at least one first detection agent that specifically binds to IL-2, or any combinations thereof.

In still other aspects, the biomarker panel can include:
A. at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-17, at least one first detection agent that specifically binds to INFγ, at least one first detection agent that specifically binds to IRA, at least one first detection agent that specifically binds to marapsin, at least one first detection agent that specifically binds to CD25, at least one first detection agent that specifically binds to HSP20, and at least one first detection agent that specifically binds to IL-23; and
B. at least one first detection agent that specifically binds to PTX-3, at least one first detection agent that specifically binds to IL-10, at least one first detection agent that specifically binds to TNFα, at least one first detection agent that specifically binds to IL-13, at least one first detection agent that specifically binds to CX3CL1, at least one first detection agent that specifically binds to CCL17, at least one first detection agent that specifically binds to CCL17, at least one first detection agent that specifically binds to IL-1β, at least one first detection agent that specifically binds to perforin, at least one first detection agent that specifically binds to CCL4, at least one first detection agent that specifically binds to GRAND B, at least one first detection agent that specifically binds to CXCL11, and at least one first detection agent that specifically binds to IL-2.

In still other aspects, the biomarker panel can include:
A. at least one first detection agent that specifically binds to IL-33, at least one first detection agent that specifically binds to CXCL10, at least one first detection agent that specifically binds to IL-27, at least one first detection agent that specifically binds to CCL24, at least one first detection agent that specifically binds to IL-6, at least one first detection agent that specifically binds to IL-17, at least one first detection agent that specifically binds to INFγ, at least one first detection agent that specifically binds to IRA, at least one first detection agent that specifically binds to marapsin, at least one first detection agent that specifically binds to CD25, at least one first detection agent that specifically binds to HSP20, and at least one first detection agent that specifically binds to IL-23;
B. at least one first detection agent that specifically binds to IL-4, at least one first detection agent that specifically binds to CCL5, at least one first detection agent that specifically binds to IL-12, at least one first detection agent that specifically binds to CCL20, at least one first detection agent that specifically binds to IL-21, at least one first detection agent that specifically binds to B7-1, at least one first detection agent that specifically binds to IL-8, at least one first detection agent that specifically binds to IL-7, or any combination thereof; and
C. at least one first detection agent that specifically binds to PTX-3, at least one first detection agent that specifically binds to IL-10, at least one first detection agent that specifically binds to TNFα, at least one first detection agent that specifically binds to IL-13, at least one first detection agent that specifically binds to CX3CL1, at least one first detection agent that specifically binds to CCL17, at least one first detection agent that specifically binds to CCL17, at least one first detection agent that specifically binds to IL-1β, at least one first detection agent that specifically binds to perforin, at least one first detection agent that specifically binds to CCL4, at least one first detection agent that specifically binds to GRAND B, at least one first detection agent that specifically binds to CXCL11, at least one first detection agent that specifically binds to IL-2, or any combinations thereof.

### MIS-C and Respiratory Syncytial Virus Biomarker Panels

In one embodiment, the biomarker panel is a panel for assessing a subject suspected of having or having multisystem inflammatory syndrome in children (MIS-C) or respiratory syncytial virus (RSV). The biomarker panel comprises a plurality of first biomarker detection agents that specifically bind to a corresponding set of first target biomarkers. The set of first target biomarkers includes Granulocyte-Macrophage Colony-Stimulating Factor (GM-CSF), Pentraxin-3 (PTX-3), marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23). Thus, the plurality of first detection agents in the biomarker panel includes at least 11 first detection agents. Specifically, the biomarker panel includes at least one first detection agent that specifically binds to GM-CSF, at least one first detection agent that specifically binds to PTX-3, at least one first detection agent that specifically binds to marapsin, at least one first detection agent that specifically binds to B7-1, at least one first detection agent that specifically binds to CCL4, at least one first detection agent that specifically binds to TREM, at least one first detection agent that specifically binds to HSP70, at least one first detection agent that specifically binds to IL-33, at least one first detection agent that specifically binds to CXCL10, at least one first detection agent that specifically binds to IL-27, and at least one first detection agent that specifically binds to IL-23. Any suitable detection agent known in the art can be used. In some aspects, the detection agent is an antibody or antigen-binding fragment thereof. In another aspect, the detection agent is an aptamer.

In some aspects, one or more of the at least one first detection agent that specifically binds to GM-CSF, at least one first detection agent that specifically binds to PTX-3, at least one first detection agent that specifically binds to marapsin, at least one first detection agent that specifically binds to B7-1, at least one first detection agent that specifically binds to CCL4, at least one first detection agent that specifically binds to TREM, at least one first detection agent that specifically binds to HSP70, at least one first detection agent that specifically binds to IL-33, at least one first detection agent that specifically binds to CXCL10, at least one first detection agent that specifically binds to IL-27, at least one first detection agent that specifically binds to IL-23 or any combination thereof is used or functions as a capture reagent in the panel. In some aspects, when at least one first detection agent that specifically binds to its corresponding target biomarker is used as a capture reagent or capture-specific binding partner, the capture reagent can optionally be immobilized on a solid support. The type of solid support is not critical and can be particle, microparticle, bead, plate, well, chip, or any combination thereof.

In some aspects, one or more of the at least one first detection agent that specifically binds to GM-CSF, at least one first detection agent that specifically binds to PTX-3, at least one first detection agent that specifically binds to marapsin, at least one first detection agent that specifically binds to B7-1, at least one first detection agent that specifically binds to CCL4, at least one first detection agent that specifically binds to TREM, at least one first detection agent that specifically binds to HSP70, at least one first detection agent that specifically binds to IL-33, at least one first detection agent that specifically binds to CXCL10, at least one first detection agent that specifically binds to IL-27, at least one first detection agent that specifically binds to IL-23, or any combination thereof is labeled with one or more detectable labels and used or functions as a detection reagent or detection specific binding partner in the panel.

In some embodiments, the set of first target biomarkers can include up to 8 additional target biomarkers. Specifically, the set of first target biomarkers can include: Granulocyte-Macrophage Colony-Stimulating Factor (GM-CSF), Pentraxin-3 (PTX-3), Marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), interleukin-23 (IL-23) and optionally, one or more of the following additional 8 target biomarkers: Perforin (Perf), C-X-C motif chemokine ligand 11 (CXCL11), Epidermal Growth Factor (EGF), interleukin-10 (IL-10), interleukin-8 (IL-8), interleukin-21 (IL-21), interleukin-2 (IL-2), immunoregulatory alpha globulin (IRA), or any combination thereof. In some aspects, all 8 of these additional target biomarkers are included in the set of first target biomarkers, or just 1, 2, 3, 4, 5, 6, or 7 of these additional target biomarkers are included.

For example, in some aspects, the biomarker panel can include at least one first detection agent that specifically binds to GM-CSF, at least one first detection agent that specifically binds to PTX-3, at least one first detection agent that specifically binds to marapsin, at least one first detection agent that specifically binds to B7-1, at least one first detection agent that specifically binds to CCL4, at least one first detection agent that specifically binds to TREM, at least one first detection agent that specifically binds to HSP70, at least one first detection agent that specifically binds to IL-33, at least one first detection agent that specifically binds to CXCL10, at least one first detection agent that specifically binds to IL-27, and at least one first detection agent that specifically binds to IL-23, and, in addition, at least one first detection agent that specifically binds to perforin, at least one first detection agent that specifically binds to CXCL11, at least one first detection agent that specifically binds to EGF, at least one first detection agent that specifically binds to IL-10, at least one first detection agent that specifically binds to IL-8, at least one first detection agent that specifically binds to IL-21, at least one first detection agent that specifically binds to IL-2, at least one first detection agent that specifically binds to IRA, or any combination thereof. Any suitable detection agent known in the art can be used in the biomarker panel. In some aspects, the detection agent is an antibody or antigen-binding fragment thereof. In another aspect, the detection agent is an aptamer. Additionally, at least one first detection agent can be used or function as a capture reagent or capture specific binding partner that is optionally immobilized on a solid support or as a detection reagent or detection specific binding partner and labeled with a detectable label.

In some aspects, the biomarker panel can include at least one first detection agent that specifically binds to GM-CSF, at least one first detection agent that specifically binds to PTX-3, at least one first detection agent that specifically binds to marapsin, at least one first detection agent that specifically binds to B7-1, at least one first detection agent that specifically binds to CCL4, at least one first detection agent that specifically binds to TREM, at least one first detection agent that specifically binds to HSP70, at least one first detection agent that specifically binds to IL-33, at least one first detection agent that specifically binds to CXCL10, at least one first detection agent that specifically binds to IL-27, and at least one first detection agent that specifically binds to IL-23, at least one first detection agent that specifically binds to perforin, at least one first detection agent that specifically binds to CXCL11, at least one first detection agent that specifically binds to EGF, at least one first detection agent that specifically binds to IL-10, at least one first detection agent that specifically binds to IL-8, at least one first detection agent that specifically binds to IL-21, at least one first detection agent that specifically binds to IL-2, and at least one first detection agent that specifically binds to IRA. In this aspect, the plurality of first detection agents in the biomarker panel will comprise at least 19 first detection agents.

In some embodiments, in addition to the first set of target biomarkers, the biomarker panel can further comprise a plurality of first biomarker detection agents that specifically bind to a corresponding set of second target biomarkers. The set of second target biomarkers includes one or more of Granzyme B (GRAND B), fractalkine (CX3CL1), C-C motif chemokine ligand 24 (CCL24), interleukin-7 (IL-7), C-C chemokine ligand 20 (CCL20), interleukin-6 (IL-6), C-C chemokine ligand 17 (CCL17), interleukin-4 (IL-4), interleukin-17 (IL-17), interleukin-15 (IL-15), tumor necrosis factor alpha (TNFα), interleukin-1β (IL-1β), interferon gamma (INFγ), interleukin-12 (IL-12) or any combinations thereof. In some aspects, all 14 of these additional target biomarkers are included in the set of second target biomarkers, or just 1, 2, 3, 4, 5, 6, 7, 8, 9, 10, 11, 12, or 13 of these second target biomarkers are included.

Thus, in some aspects, the biomarker panel can include:
A. at least one first detection agent that specifically binds to GM-CSF, at least one first detection agent that specifically binds to PTX-3, at least one first detection agent that specifically binds to marapsin, at least one first detection agent that specifically binds to B7-1, at least one first detection agent that specifically binds to CCL4, at least one first detection agent that specifically binds to TREM, at least one first detection agent that specifically binds to HSP70, at least one first detection agent that specifically binds to IL-33, at least one first detection agent that specifically binds to CXCL10, at least one first detection agent that specifically binds to IL-27, and at least one first detection agent that specifically binds to IL-23; and
B. at least one first detection agent that specifically binds to perforin, at least one first detection agent that specifically binds to CXCL11, at least one first detection agent that specifically binds to EGF, at least one first detection agent that specifically binds to IL-10, at least one first detection agent that specifically binds to IL-8, at least one first detection agent that specifically binds to IL-21, at least one first detection agent that specifically binds to IL-2, at least one first detection agent that specifically binds to IRA, or any combination thereof. Any suitable detection agent known in the art can be used in the biomarker panel. In some aspects, the detection agent is an antibody or antigen-binding fragment thereof. In another aspect, the detection agent is an aptamer. Additionally, the at least one first detection agent can be used or function as a capture reagent or capture specific binding partner that is optionally immobilized on a solid support or as a detection reagent or detection specific binding partner and labeled with a detectable label.

In still yet other aspects, the biomarker panel can include:
A. at least one first detection agent that specifically binds to GM-CSF, at least one first detection agent that specifically binds to PTX-3, at least one first detection agent that specifically binds to marapsin, at least one first detection agent that specifically binds to B7-1, at least one first detection agent that specifically binds to CCL4, at least one first detection agent that specifically binds to TREM, at least one first detection agent that specifically binds to HSP70, at least one first detection agent that specifically binds to IL-33, at least one first detection agent that specifically binds to CXCL10, at least one first detection agent that specifically binds to IL-27, and at least one first detection agent that specifically binds to IL-23; and
B. at least one first detection agent that specifically binds to perforin, at least one first detection agent that specifically binds to CXCL11, at least one first detection agent that specifically binds to EGF, at least one first detection agent that specifically binds to IL-10, at least one first detection agent that specifically binds to IL-8, at least one first detection agent that specifically binds to IL-21, at least one first detection agent that specifically binds to IL-2, and at least one first detection agent that specifically binds to IRA. Any suitable detection agent known in the art can be used in the biomarker panel. In some aspects, the detection agent is an antibody or antigen-binding fragment thereof. In another aspect, the detection agent is an aptamer. Additionally, the at least one first detection agent can be used or function as a capture reagent or capture specific binding partner that is optionally immobilized on a solid support or as a detection reagent or detection specific binding partner and labeled with a detectable label.

In still yet other aspects, the biomarker panel can include:
A. at least one first detection agent that specifically binds to GM-CSF, at least one first detection agent that specifically binds to PTX-3, at least one first detection agent that specifically binds to marapsin, at least one first detection agent that specifically binds to B7-1, at least one first detection agent that specifically binds to CCL4, at least one first detection agent that specifically binds to TREM, at least one first detection agent that specifically binds to HSP70, at least one first detection agent that specifically binds to IL-33, at least one first detection agent that specifically binds to CXCL10, at least one first detection agent that specifically binds to IL-27, and at least one first detection agent that specifically binds to IL-23; and
B. at least one first detection agent that specifically binds to GRAND B, at least one first detection agent that specifically binds to CX3CL1, at least one first detection agent that specifically binds to CCL24, at least one first detection agent that specifically binds to IL-7, at least one first detection agent that specifically binds to CCL20, at least one first detection agent that specifically binds to IL-6, at least one first detection agent that specifically binds to CCL17, at least one first detection agent that specifically binds to IL-4, at least one first detection agent that specifically binds to IL-17, at least one first detection agent that specifically binds to IL-15, at least one first detection agent that specifically binds to TNFα, at least one first detection agent that specifically binds to IL-1β, at least one first detection agent that specifically binds to INFγ, at least one first detection agent that specifically binds to IL-12, or any combination thereof. Any suitable detection agent known in the art can be used in the biomarker panel. In some aspects, the detection agent is an antibody or antigen-binding fragment thereof. In another aspect, the detection agent is an aptamer. Additionally, the at least one first detection agent can be used or function as a capture reagent or capture specific binding partner that is optionally immobilized on a solid support or as a detection reagent or detection specific binding partner and labeled with a detectable label.

In yet still other aspects, the biomarker panel can include:
A. at least one first detection agent that specifically binds to GM-CSF, at least one first detection agent that specifically binds to PTX-3, at least one first detection agent that specifically binds to marapsin, at least one first detection agent that specifically binds to B7-1, at least one first detection agent that specifically binds to CCL4, at least one first detection agent that specifically binds to TREM, at least one first detection agent that specifically binds to HSP70, at least one first detection agent that specifically binds to IL-33, at least one first detection agent that specifically binds to CXCL10, at least one first detection agent that specifically binds to IL-27, and at least one first detection agent that specifically binds to IL-23; and
B. at least one first detection agent that specifically binds to GRAND B, at least one first detection agent that specifically binds to CX3CL1, at least one first detection agent that specifically binds to CCL24, at least one first detection agent that specifically binds to IL-7, at least one first detection agent that specifically binds to CCL20, at least one first detection agent that specifically binds to IL-6, at least one first detection agent that specifically binds to CCL17, at least one first detection agent that specifically binds to IL-4, at least one first detection agent that specifically binds to IL-17, at least one first detection agent that specifically binds to IL-15, at least one first detection agent that specifically binds to TNFα, at least one first detection agent that specifically binds to IL-1β, at least one first detection agent that specifically binds to INFγ, and at least one first detection agent that specifically binds to IL-12. Any suitable detection agent known in the art can be used in the biomarker panel. In some aspects, the detection agent is an antibody or antigen-binding fragment thereof. In another aspect, the detection agent is an aptamer. Additionally, the at least one first detection agent can be used or function as a capture reagent or capture specific binding partner that is optionally immobilized on a solid support or as a detection reagent or detection specific binding partner and labeled with a detectable label.

In still yet other aspects, the biomarker panel can include:
A. at least one first detection agent that specifically binds to GM-CSF, at least one first detection agent that specifically binds to PTX-3, at least one first detection agent that specifically binds to marapsin, at least one first detection agent that specifically binds to B7-1, at least one first detection agent that specifically binds to CCL4, at least one first detection agent that specifically binds to TREM, at least one first detection agent that specifically binds to HSP70, at least one first detection agent that specifically binds to IL-33, at least one first detection agent that specifically binds to CXCL10, at least one first detection agent that specifically binds to IL-27, and at least one first detection agent that specifically binds to IL-23;
B. at least one first detection agent that specifically binds to perforin, at least one first detection agent that specifically binds to CXCL11, at least one first detection agent that specifically binds to EGF, at least one first detection agent that specifically binds to IL-10, at least one first detection agent that specifically binds to IL-8, at least one first detection agent that specifically binds to IL-21, at least one first detection agent that specifically binds to IL-2, at least one first detection agent that specifically binds to IRA, or any combination thereof; and
C. at least one first detection agent that specifically binds to GRAND B, at least one first detection agent that specifically binds to CX3CL1, at least one first detection agent that specifically binds to CCL24, at least one first detection agent that specifically binds to IL-7, at least one first detection agent that specifically binds to CCL20, at least one first detection agent that specifically binds to IL-6, at least one first detection agent that specifically binds to CCL17, at least one first detection agent that specifically binds to IL-4, at least one first detection agent that specifically binds to IL-17, at least one first detection agent that specifically binds to IL-15, at least one first detection agent that specifically binds to TNFα, at least one first detection agent that specifically binds to IL-1β, at least one first detection agent that specifically binds to INFγ, at least one first detection agent that specifically binds to IL-12, or any combination thereof. Any suitable detection agent known in the art can be used in the biomarker panel. In some aspects, the detection agent is an antibody or antigen-binding fragment thereof. In another aspect, the detection agent is an aptamer. Additionally, the at least one first detection agent can be used or function as a capture reagent or capture specific binding partner that is optionally immobilized on a solid support or as a detection reagent or detection specific binding partner and labeled with a detectable label.

In other embodiments, any of the above-described biomarker panels can be provided in the form of a chip, such as a grating-coupled fluorescent plasmonic biosensor chip. In some aspects, the chip contains a plurality of first detection agents that specifically bind to each corresponding target biomarker (e.g., a first target biomarker or a first target biomarker and a second target biomarker).

In still yet other embodiments, any of the above-described biomarker panels can be configured as a multiplex assay or as an ELISA assay. These biomarker panels can be used in the methods, devices, and kits described in the sections below.

### III. Methods of Assessing Subjects Suspected of Having Multisystem Inflammatory Syndrome in Children, Kawasaki Disease, SARS-CoV-2, Respiratory Syncytial Virus, an Adenovirus, Influenza A, B, C, or D, and/or Parainfluenza

In another embodiment, the present disclosure relates to methods for assessing whether a subject is suspected of having or has multisystem inflammatory syndrome in children, Kawasaki disease, SARS-CoV-2, respiratory syncytial virus, an adenovirus, influenza A, B, C, or D, or parainfluenza. In some aspects, the subject is a mammal. In some aspects, the subject is a human pediatric subject. In some aspects, the subject is a human adult subject. In other aspects, the subject is a dog. In still other aspects, the subject is a cat. In still other aspects, the subject is a cow or cattle. In still other aspects, the subject is a bird. In still further aspects, the subject is a pig. In some aspects, the biomarker panels described in Section II (“Biomarker Panels”) are used to determine or predict whether a subject (e.g., such as a pediatric subject) is at risk of developing multisystem inflammatory syndrome in children, Kawasaki disease, SARS-CoV-2, respiratory syncytial virus, an adenovirus, influenza A, B, C, or D, or parainfluenza. In yet other aspects, the biomarker panels are used to determine or diagnose that a subject (e.g., such as a pediatric subject) has multisystem inflammatory syndrome in children, Kawasaki disease, SARS-CoV-2, respiratory syncytial virus, an adenovirus, influenza A, B, C, or D, or parainfluenza.

In one embodiment, the method involves obtaining a sample from a subject suspected of having or having multisystem inflammatory syndrome in children, Kawasaki disease, SARS-CoV-2, respiratory syncytial virus, an adenovirus, influenza A, B, C, or D, or parainfluenza. In some aspects, the subject is a human pediatric subject. In other aspects, the subject is a human adult subject. In some aspects, the sample is a blood sample. In other aspects, the sample is a plasma sample. In yet other aspects, the sample is a serum sample. In still other aspects, the sample is a salvia sample. In some aspects, the sample is a blood sample obtained from a human pediatric subject. In other aspects, the sample is a plasma sample obtained from a human pediatric subject. In yet other aspects, the sample is a serum sample obtained from a human pediatric subject. In still other aspects, the sample is a salvia sample obtained from a human pediatric subject.

Once a sample is obtained from a subject, the sample is contacted with one or more of the biomarker panels described in Section II (“Biomarker Panels”) herein. As mentioned in Section II (“Biomarker Panels”), the biomarker panel contains a plurality of first biomarker detection reagents specific to a corresponding target biomarker. In some aspects, each of the plurality of first biomarker detection reagents is specific for (e.g., specifically binds) a corresponding target biomarker in a set of first or first and second biomarkers, which are shown in the below Tables A, B and C.

TABLE A
Biomarker Set to Distinguish MIS-C from Kawasaki Disease

| | | Higher Relative | Lower Relative |
|---|---|---|---|
| | | Intensity than | Intensity than |
| Set of Target | | Kawasaki | Kawasaki |
| Biomarkers | Biomarker | Disease | Disease |
| 1 | PTX-3 | X | |
| 1 | IL-10 | X | |
| 1 | IL-33 | X | |
| 1 | CXCL10 | X | |
| 1 | IL-27 | X | |
| 1 | CCL24 | X | |
| 1 | IL-6 | X | |
| 1 | IL-4 | X | |
| 1 | IL-17 | X | |
| 1 | CCL5 | X | |
| 1 | TNFα | X | |
| 1 | IL-13 | X | |
| 1 | INFy | X | |
| 1 | IL-12 | X | |
| 1 | IRA | X | |
| 1A* | B7-1 | | X |
| 1A | CXCL11 | | X |
| 1A | IL-8 | X | |
| 1A | IL-7 | X | |
| 1A | IL-23 | X | |
| 1A | IL-2 | X | |
| 1A | IL-15 | X | |
| 2 | Marapsin | | X |
| 2 | CX3CL1 | | X |
| 2 | CCL20 | | X |
| 2 | IL-21 | X | |
| 2 | CCL17 | X | |
| 2 | IL-1B | X | |

*Biomarkers in 1A are can be included in set 1 if desired.

TABLE B
Biomarker Set to Distinguish MIS-C from Severe COVID-19

| | | Higher Relative | Lower Relative |
|---|---|---|---|
| Set of Target | | Intensity than | Intensity than |
| Biomarkers | Biomarkers | Severe COVID | Severe COVID |
| 1 | IL-33 | X | |
| 1 | CXCL10 | X | |
| 1 | IL-27 | X | |
| 1 | CCL24 | X | |
| 1 | IL-6 | X | |
| 1 | IL-17 | X | |
| 1 | INFy | X | |
| 1 | IRA | X | |
| 1 | Marapsin | X | |
| 1 | CD25 | X | |
| 1 | HSP70 | X | |
| 1 | IL-23 | X | |
| 1A* | IL-4 | X | |
| 1A | CCL5 | X | |
| 1A | IL-12 | X | |
| 1A | CCL20 | X | |
| 1A | IL-21 | X | |
| 1A | B7-1 | X | |
| 1A | IL-8 | X | |
| 1A | IL-7 | X | |
| 2 | PTX-3 | X | |
| 2 | IL-10 | X | |
| 2 | TNFα | X | |
| 2 | IL-13 | X | |
| 2 | CX3CL1 | X | |
| 2 | CCL17 | X | |
| 2 | IL-1B | X | |
| 2 | Perforin | X | |
| 2 | CCL4 | X | |
| 2 | Grand B | X | |
| 2 | CXCL11 | X | |
| 2 | IL-2 | X | |

Biomarkers in 1A are can be included in set 1 if desired.

TABLE C
Biomarker Set to Distinguish MIS-C from RSV

| | | Higher Relative | Lower Relative |
|---|---|---|---|
| Set of Target | | Intensity than | Intensity than |
| Biomarkers | Biomarker | RSV | RSV |
| 1 | GM-CSF | X | |
| 1 | PTX-3 | X | |
| 1 | Marapsin | X | |
| 1 | B7-1 | X | |
| 1 | CCL4 | X | |
| 1 | TREM | X | |
| 1 | HSP70 | X | |
| 1 | IL-33 | X | |
| 1 | CXCL10 | X | |
| 1 | IL-27 | X | |
| 1 | IL-23 | | X |
| 1A* | Perforin | X | |
| 1A | CXCL11 | X | |
| 1A | EGF | X | |
| 1A | IL-10 | | X |
| 1A | IL-8 | X | |
| 1A | IL-21 | | X |
| 1A | IL-2 | | X |
| 1A | IRA | X | |
| 2 | Grand B | X | |
| 2 | CX3CL1 | | X |
| 2 | CCL24 | X | |
| 2 | IL-7 | X | |
| 2 | CCL20 | X | |
| 2 | IL-6 | X | |
| 2 | CCL17 | | X |
| 2 | IL-4 | X | |
| 2 | IL-17 | X | |
| 2 | IL-15 | | X |
| 2 | TNFα | X | |
| 2 | IL-1B | | X |
| 2 | INFy | X | |
| 2 | IL-12 | | X |

*Biomarkers in 1A are can be included in set 1 if desired.

After the sample is in contact with the biomarker panel, the amount or concentration of each of the target biomarkers is measured, determined, and obtained using routine techniques known in the art. Determining the amount or concentration of each of the target biomarkers may be carried out by any suitable technique that allows for the amount or concentration of each target biomarker to be determined. In some aspects, the amount or concentration of a target biomarker in the methods described herein can be determined by immunoassays using sandwich, competition, or other assay formats. These assays will develop a signal which is indicative for the presence or absence or the amount of a target biomarker. Additional methods that can be used comprise measuring a physical or chemical property specific to the target biomarker, such as its precise molecular mass or NMR spectrum. Said methods include biosensors, optical devices coupled to immunoassays, biochips, analytical devices such as mass-spectrometers, NMR analyzers, surface plasmon resonance measurement equipment, or chromatography devices. Further, methods include micro-plate ELISA-based methods, fully automated or robotic immunoassays. Additional measurement methods that can be used may also include precipitation (particularly immunoprecipitation), electrochemiluminescence (electro-generated chemiluminescence), RIA (radioimmunoassay), ELISA (enzyme-linked immunosorbent assay), electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art such as gel electrophoresis, 2D gel electrophoresis, SDS polyacrylamide gel electrophoresis (SDS-PAGE) or Western Blotting.

Once the amount or concentration of each target biomarker is determined, the relative intensity value for each target biomarker (e.g., first target biomarker, second target biomarker, or first and second target biomarker) is determined based on the amount or concentration previously determined. The relative intensity value for each target biomarker (e.g., first target biomarker, second target biomarker, or first and second target biomarker), can be determined using routine techniques known in the art.

Once the relative intensity value for each of the first target biomarkers, each of the second target biomarkers, or each of the first and second target biomarkers is determined, the relative intensity values calculated are compared to one or more reference values for the target first biomarkers, second biomarkers, or first and second biomarkers. In some aspects, once the relative intensity values for each of the first target biomarkers, second target biomarkers, or first and second target biomarkers is determined, the relative intensity values of all the target biomarkers is compared to a reference value or reference value range for the corresponding biomarkers. Once this comparison is completed, an assessment of the subject is made.

In some embodiments, using the information provided in Table A, an assessment is made to determine whether a subject is or is not at risk of developing multisystem inflammatory syndrome in children or Kawasaki disease. In some aspects, the method involves predicting whether a subject is or is not at risk of developing multisystem inflammatory syndrome in children or Kawasaki disease. In still other embodiments, an assessment is made that a subject has or does not have multisystem inflammatory syndrome in children or Kawasaki disease. In some aspects, the method involves diagnosing a subject as having or not having multisystem inflammatory syndrome in children or Kawasaki disease.

In some embodiments, the method involves measuring a panel of first target biomarkers in a sample obtained from a subject suspected of having multisystem inflammatory syndrome in children or Kawasaki disease. In some aspects, the subject is a human pediatric subject. The measurement involves determining the amount of a set of first target biomarkers, where the target biomarkers comprise pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12) and immunoregulatory alpha globulin (IRA). As discussed previously herein in Section II (“Biomarker Panels”), the panel comprises a plurality of first biomarker detection agents, which each specifically bind to a corresponding target biomarker. Once the amount or concentration of each target biomarker is determined, the relative intensity value for each biomarker is calculated using routine techniques known in the art. In some aspects, once the relative intensity value for each of the target biomarkers is determined, the relative intensity values of all the target biomarkers is compared to a reference value or reference value range for the corresponding biomarkers. Once this comparison is completed, an assessment of the subject is made or determined. For example, if one or more of the target biomarkers comprising pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12) or immunoregulatory alpha globulin (IRA) have a relative intensity higher than the reference values, a determination may be made that the subject has MIS-C. If one or more of the target biomarkers comprising pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12) or immunoregulatory alpha globulin (IRA) has a relative intensity value lower than the reference values, then a determination may be made that the subject does not have MIS-C but may have Kawasaki disease.

In some aspects of the above method, a panel of first target biomarkers for use is assessing a subject suspected of MIS-C or Kawasaki disease comprises Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12) and immunoregulatory alpha globulin (IRA). In some aspects, the panel of first target biomarkers for use in assessing a subject suspected of MIS-C or Kawasaki disease comprises Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-2 (IL-12) and immunoregulatory alpha globulin (IRA) and optionally, one or more of B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof. In aspects where the target biomarkers B7-1 and CXCL11 are included in the method, if the relative intensity calculated for B7-1 and/or CXCL11 is lower than the reference values, then a determination may be made that the subject may have MIS-C. If the target biomarkers B7-1 and/or CXCL11 is higher than the reference value, then a determination may be made that the subject may have Kawasaki disease.

In some additional embodiments, the method further involves measuring a panel of first target biomarkers and second target biomarkers in a sample obtained from a subject suspected of having or having multisystem inflammatory syndrome in children or Kawasaki disease. In some aspects, the subject is a human pediatric subject. The measurement involves determining the amount or concentration of a set of first target biomarkers and a set of second target biomarkers. The set of first target biomarkers comprises pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), and immunoregulatory alpha globulin (IRA) and optionally, one or more of B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof. The set of second target biomarkers comprises marapsin, fractalkine (CX3CL1), C-C chemokine ligand 20 (CCL20), interleukin-21, C-C chemokine ligand 17 (CCL17), interleukin-1β (IL-1β), or any combination thereof.

In some aspects, the set of first target biomarkers comprises pentraxin-3 (PTX-3), Interleukin-10 (IL-10), Interleukin-33 (IL-33), C-X-C motif chemokine ligand 10 (CXCL10), Interleukins-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), and immunoregulatory alpha globulin (IRA), and optionally, one or more of B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), and interleukin-15 (IL-15), or combinations thereof. The set of second target biomarkers comprises one or more of marapsin, fractalkine (CX3CL1), C-C chemokine ligand 20 (CCL20), interleukin-21, C-C chemokine ligand 17 (CCL17), interleukin-1β (IL-1β), or any combination thereof. As discussed previously herein in Section II (“Biomarker Panels”), the panel comprises a plurality of first biomarker detection agents, which each specifically bind to a corresponding first target biomarker and second target biomarker. Once the amount or concentration of each of the first target biomarkers and each of the second target biomarkers is determined, the relative intensity of each first target biomarker and the relative intensity of each second target biomarker is calculated using routine techniques known in the art. In some aspects, once the relative intensity values for each of the first target biomarkers and each of the second target biomarkers is determined, the relative intensity values of all of the first target biomarkers and second target biomarkers is compared to reference values or reference value ranges for the corresponding first and second target biomarkers. Once this comparison is completed, an assessment of the subject is made or determined. A subject may be determined to have MIS-C if: the relative intensities of the biomarkers pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), immunoregulatory alpha globulin (IRA) are higher than the reference values; and (a) one or more relative intensities of the biomarkers interleukin-8 (IL-8), interleukin-7 (IL-7), interleukin-23 (IL-23), interleukin-2 (IL-2), and/or interleukin-15 (IL-15) are higher than the reference levels; (b) one or more of the relative intensities of the biomarkers interleukin-21, C-C chemokine ligand 17 (CCL17), and/or interleukin-1β (IL-1β) are higher than the reference levels; (c) one or more relative intensities of B-lymphocyte activation antigen B7 (B7-1) and/or C-X-C motif chemokine ligand 11 (CXCL11) are lower than the reference levels; (d) one or more relative intensities of marapsin, fractalkine (CX3CL1), and/or C-C chemokine ligand 20 (CCL20) are lower than the reference levels; or (e) any combination of (a)-(d). A subject may be determined to have Kawasaki disease if: the relative intensities of the biomarkers pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), immunoregulatory alpha globulin (IRA) are higher than the reference values; and (a) one or more relative intensities of the biomarkers interleukin-8 (IL-8), interleukin-7 (IL-7), interleukin-23 (IL-23), interleukin-2 (IL-2), and/or interleukin-15 (IL-15) are lower than the reference levels; (b) one or more of the relative intensities of the biomarkers interleukin-21, C-C chemokine ligand 17 (CCL17), and/or interleukin-1β (IL-1β) are lower than the reference levels; (c) one or more relative intensities of B-lymphocyte activation antigen B7 (B7-1) and/or C-X-C motif chemokine ligand 11 (CXCL11) are higher than the reference levels; (d) one or more relative intensities of marapsin, fractalkine (CX3CL1), and/or C-C chemokine ligand 20 (CCL20) are higher than the reference levels; or (e) any combination of (a)-(d).

In some embodiments, using the information provided in Table B, an assessment is made to determine whether a subject is or is not at risk of developing multisystem inflammatory syndrome in children, Severe Acute Respiratory Syndrome Coronavirus 2 (SARS-CoV-2), or multisystem inflammatory syndrome in children and Severe Acute Respiratory Syndrome Coronavirus 2 (SARS-CoV-2). In some aspects, the method involves predicting whether a subject is or is not at risk of developing multisystem inflammatory syndrome in children, Severe Acute Respiratory Syndrome Coronavirus 2 (SARS-CoV-2), or multisystem inflammatory syndrome in children and Severe Acute Respiratory Syndrome Coronavirus 2 (SARS-CoV-2). In still other embodiments, an assessment is made that a subject has or does not have multisystem inflammatory syndrome in children, Severe Acute Respiratory Syndrome Coronavirus 2 (SARS-CoV-2), or multisystem inflammatory syndrome in children and Severe Acute Respiratory Syndrome Coronavirus 2 (SARS-CoV-2). In some aspects, the method involves diagnosing a subject as having or not having multisystem inflammatory syndrome in children, Severe Acute Respiratory Syndrome Coronavirus 2 (SARS-CoV-2), or multisystem inflammatory syndrome in children and Severe Acute Respiratory Syndrome Coronavirus 2 (SARS-CoV-2).

In some embodiments, the method involves measuring a panel of first target biomarkers in a sample obtained from a subject suspected of having multisystem inflammatory syndrome in children, Severe Acute Respiratory Syndrome Coronavirus 2 (SARS-CoV-2), or multisystem inflammatory syndrome in children and Severe Acute Respiratory Syndrome Coronavirus 2 (SARS-CoV-2). In some aspects, the subject is a human pediatric subject. The measurement involves determining the amount of a set of first target biomarkers, where the target biomarkers comprise Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), and interleukin-23 (IL-23). As discussed previously herein in Section II (“Biomarker Panels”), the panel comprises a plurality of first biomarker detection agents, which each specifically bind to a corresponding target biomarker. Once the amount or concentration of each target biomarker is determined, the relative intensity value for each biomarker is calculated using routine techniques known in the art. In some aspects, once the relative intensity value for each of the target biomarkers is determined, the relative intensity values of all the target biomarkers is compared to a reference value or reference value range for the corresponding biomarkers. Once this comparison is completed, an assessment of the subject is made or determined. For example, if one or more of the target biomarkers comprising Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), or interleukin-23 (IL-23) have a relative intensity higher than the reference values, a determination may be made that the subject has MIS-C. If one or more of the target biomarkers comprising Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), or interleukin-23 (IL-23) has a relative intensity value lower than the reference values, then a determination may be made that the subject does not have MIS-C but may have Severe Acute Respiratory Syndrome Coronavirus 2 (SARS-CoV-2).

In some aspects of the above method, a panel of first target biomarkers for use is assessing a subject suspected of MIS-C or Severe Acute Respiratory Syndrome Coronavirus 2 (SARS-CoV-2) comprises Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), and interleukin-23 (IL-23). In some aspects, the panel of first target biomarkers for use in assessing a subject suspected of MIS-C or Severe Acute Respiratory Syndrome Coronavirus 2 (SARS-CoV-2) comprises Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), marapsin, interleukin-2 receptor alpha chain (CD25), Heat Shock Protein 70 (HSP70), and interleukin-23 (IL-23) and optionally, one or more of Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7) or any combination thereof. In some aspects, were one or more of the target biomarkers Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7) or any combination thereof are higher than the reference values, then a determination may be made that the subject may have MIS-C. If one or more of the target biomarkers Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7) or any combination thereof is lower than the reference value, then a determination may be made that the subject may have Severe Acute Respiratory Syndrome Coronavirus 2 (SARS-CoV-2).

In some additional embodiments, the method further involves measuring a panel of first target biomarkers and second target biomarkers in a sample obtained from a subject suspected of having or having multisystem inflammatory syndrome in children, Severe Acute Respiratory Syndrome Coronavirus 2 (SARS-CoV-2), or multisystem inflammatory syndrome in children and Severe Acute Respiratory Syndrome Coronavirus 2 (SARS-CoV-2). In some aspects, the subject is a human pediatric subject. The measurement involves determining the amount or concentration of a set of first target biomarkers and a set of second target biomarkers. The set of first target biomarkers comprise Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), marapsin, interleukin-2 receptor alpha chain (CD25), Heat Shock Protein 70 (HSP70), and interleukin-23 (IL-23) and optionally, one or more of Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7), or any combination thereof. The set of second target biomarkers comprises Pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2) or any combination thereof.

In some aspects, the set of first target biomarkers comprises Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), marapsin, interleukin-2 receptor alpha chain (CD25), Heat Shock Protein 70 (HSP70), and interleukin-23 (IL-23) and optionally, one or more of Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7), or any combination thereof. The set of second target biomarkers comprises: pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2) or any combination thereof As discussed previously herein in Section II (“Biomarker Panels”), the panel comprises a plurality of first biomarker detection agents, which each specifically bind to a corresponding first target biomarker and second target biomarker. Once the amount or concentration of each of the first target biomarkers and each of the second target biomarkers is determined, the relative intensity of each first target biomarker and the relative intensity of each second target biomarker is calculated using routine techniques known in the art. In some aspects, once the relative intensity values for each of the first target biomarkers and each of the second target biomarkers is determined, the relative intensity values of all of the first target biomarkers and second target biomarkers is compared to reference values or reference value ranges for the corresponding first and second target biomarkers. Once this comparison is completed, an assessment of the subject is made or determined. A subject may be determined to have MIS-C if: the relative intensities of the Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), marapsin, interleukin-2 receptor alpha chain (CD25), Heat Shock Protein 70 (HSP70), and interleukin-23 (IL-23) are higher than the reference values; and (a) one or more of the relative intensities of the biomarkers Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7) are higher than the reference values; (b) one or more relative intensities of pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1p (IL-1β), Perforin (Perf), C-C Motif Chemokine Ligand 4 (CCL4), Granzyme B (GRAND B), C-X-C motif chemokine ligand 11 (CXCL11), interleukin-2 (IL-2) are higher than the reference values; or (d) any combination of (a)-(b). A subject may be determined to have Severe Acute Respiratory Syndrome Coronavirus 2 (SARS-CoV-2) if: the relative intensities of the Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), marapsin, interleukin-2 receptor alpha chain (CD25), Heat Shock Protein 70 (HSP70), and interleukin-23 (IL-23) are lower than the reference values; and (a) one or more of the relative intensities of the biomarkers Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7) are lower than the reference values; (b) one or more relative intensities of pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2) are lower than the reference values; or (c) any combination of (a)-(b).

In some embodiments, using the information provided in Table C, an assessment is made to determine whether a subject is or is not at risk of developing multisystem inflammatory syndrome in children, Respiratory syncytial virus, or multisystem inflammatory syndrome in children and respiratory syncytial virus. In some aspects, the method involves predicting whether a subject is or is not at risk of developing multisystem inflammatory syndrome in children, respiratory syncytial virus, or multisystem inflammatory syndrome in children and respiratory syncytial virus. In still other embodiments, an assessment is made that a subject has or does not have multisystem inflammatory syndrome in children, Respiratory syncytial virus, or multisystem inflammatory syndrome in children and respiratory syncytial virus. In some aspects, the method involves diagnosing a subject as having or not having multisystem inflammatory syndrome in children, respiratory syncytial virus, or multisystem inflammatory syndrome in children and respiratory syncytial virus.

In some embodiments, the method involves measuring a panel of first target biomarkers in a sample obtained from a subject suspected of having multisystem inflammatory syndrome in children, respiratory syncytial virus, or multisystem inflammatory syndrome in children and respiratory syncytial virus. In some aspects, the subject is a human pediatric subject. The measurement involves determining the amount of a set of first target biomarkers, where the target biomarkers comprise Granulocyte-Macrophage Colony-Stimulating Factor (GM-CSF), Pentraxin-3 (PTX-3), Marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23). As discussed previously herein in Section II (“Biomarker Panels”), the panel comprises a plurality of first biomarker detection agents, which each specifically bind to a corresponding target biomarker. Once the amount or concentration of each target biomarker is determined, the relative intensity value for each biomarker is calculated using routine techniques known in the art. In some aspects, once the relative intensity value for each of the target biomarkers is determined, the relative intensity values of all the target biomarkers is compared to a reference value or reference value range for the corresponding biomarkers. Once this comparison is completed, an assessment of the subject is made or determined. For example, if one or more of the target biomarkers comprising comprise Granulocyte-Macrophage Colony-Stimulating Factor (GM-CSF), Pentraxin-3 (PTX-3), marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), or interleukin-27 (IL-27), have a relative intensity higher than the reference values and/or the relative intensity of interleukin-23 (IL-23) is lower than the reference value, a determination may be made that the subject has MIS-C. If one or more of the target biomarkers comprising one or more of the target biomarkers comprising comprise Granulocyte-Macrophage Colony-Stimulating Factor (GM-CSF), Pentraxin-3 (PTX-3), marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), or interleukin-27 (IL-27), have a relative intensity lower than the reference values and/or the relative intensity of interleukin-23 (IL-23) is higher than the reference value then a determination may be made that the subject does not have MIS-C but may have respiratory syncytial virus.

In some aspects of the above method, a panel of first target biomarkers for use is assessing a subject suspected of MIS-C or respiratory syncytial virus comprises Granulocyte-Macrophage Colony-Stimulating Factor (GM-CSF), Pentraxin-3 (PTX-3), Marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23). In some aspects, the panel of first target biomarkers for use in assessing a subject suspected of MIS-C or respiratory syncytial virus comprises Granulocyte-Macrophage Colony-Stimulating Factor (GM-CSF), Pentraxin-3 (PTX-3), Marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), interleukin-23 (IL-23) and optionally, one or more of perforin (Perf), C-X-C motif chemokine ligand 11 (CXCL11), Epidermal Growth Factor (EGF), interleukin-10 (IL-10), interleukin-8 (IL-8), interleukin-21 (IL-21), interleukin-2 (IL-2), immunoregulatory alpha globulin (IRA), or any combination thereof. In aspects where the target biomarkers IL-10, IL-21 and/or IL-2 are included in the method, if the relative intensity calculated for IL-10, IL-21 and/or IL-2 is lower than the reference values, then a determination may be made that the subject may have MIS-C. If the target biomarkers IL-10, IL-21 and/or IL-2 is higher than the reference value, then a determination may be made that the subject may have Respiratory syncytial virus.

In some additional embodiments, the method further involves measuring a panel of first target biomarkers and second target biomarkers in a sample obtained from a subject suspected of having or having multisystem inflammatory syndrome in children, Respiratory syncytial virus, or multisystem inflammatory syndrome in children and Respiratory syncytial virus. In some aspects, the subject is a human pediatric subject. The measurement involves determining the amount or concentration of a set of first target biomarkers and a set of second target biomarkers. The set of first target biomarkers comprises Granulocyte-Macrophage Colony-Stimulating Factor (GM-CSF), Pentraxin-3 (PTX-3), marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23) and optionally, one or more of perforin (Perf), C-X-C motif chemokine ligand 11 (CXCL11), Epidermal Growth Factor (EGF), interleukin-10 (IL-10), interleukin-8 (IL-8), interleukin-21 (IL-21), interleukin-2 (IL-2), immunoregulatory alpha globulin (IRA), or any combination thereof.

In some aspects, the set of first target biomarkers comprises Granulocyte-Macrophage Colony-Stimulating Factor (GM-CSF), Pentraxin-3 (PTX-3), marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23) and optionally, one or more of perforin (Perf), C-X-C motif chemokine ligand 11 (CXCL11), Epidermal Growth Factor (EGF), interleukin-10 (IL-10), interleukin-8 (IL-8), interleukin-21 (IL-21), interleukin-2 (IL-2), immunoregulatory alpha globulin (IRA), or any combination thereof. The set of second target biomarkers comprises one or more of Granzyme B (GRAND B), fractalkine (CX3CL1), C-C motif chemokine ligand 24 (CCL24), interleukin-7 (IL-7), C-C chemokine ligand 20 (CCL20), interleukin-6 (IL-6), C-C chemokine ligand 17 (CCL17), interleukin-4 (IL-4), interleukin-17 (IL-17), interleukin-15 (IL-15), tumor necrosis factor alpha (TNFα), interleukin-1β (IL-1β), interferon gamma (INFγ), interleukin-12 (IL-12), or any combination thereof. As discussed previously herein in Section II (“Biomarker Panels”), the panel comprises a plurality of first biomarker detection agents, which each specifically bind to a corresponding first target biomarker and second target biomarker. Once the amount or concentration of each of the first target biomarkers and each of the second target biomarkers is determined, the relative intensity of each first target biomarker and the relative intensity of each second target biomarker is calculated using routine techniques known in the art. In some aspects, once the relative intensity values for each of the first target biomarkers and each of the second target biomarkers is determined, the relative intensity values of all of the first target biomarkers and second target biomarkers is compared to reference values or reference value ranges for the corresponding first and second target biomarkers. Once this comparison is completed, an assessment of the subject is made or determined. A subject may be determined to have MIS-C if: the relative intensities of the biomarkers Granulocyte-Macrophage Colony-Stimulating Factor (GM-CSF), Pentraxin-3 (PTX-3), marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), or interleukin-27 (IL-27) are higher than the reference values and the relative intensity of interleukin-23 (IL-23) is lower than the reference values; and (a) one or more relative intensities of the biomarkers perforin (Perf), C-X-C motif chemokine ligand 11 (CXCL11), Epidermal Growth Factor (EGF), interleukin-8 (IL-8) and/or immunoregulatory alpha globulin (IRA) are higher than the reference values; (b) one or more of the relative intensities of the biomarkers interleukin-10 (IL-10), interleukin-21 (IL-21), and/or interleukin-2 (IL-2) are lower than the reference values; (c) one or more relative intensities Granzyme B (GRAND B), C-C motif chemokine ligand 24 (CCL24), interleukin-7 (IL-7), C-C chemokine ligand 20 (CCL20), interleukin-6 (IL-6), interleukin-4 (IL-4), interleukin-17 (IL-17), tumor necrosis factor alpha (TNFα), and/or interferon gamma (INFγ) are higher than the reference values; (d) one or more relative intensities of fractalkine (CX3CL1), C-C chemokine ligand 17 (CCL17), interleukin-15 (IL-15), interleukin-1β (IL-1β), and/or interleukin-12 (IL-12) are lower than the reference values; or (e) any combination of (a)-(d). A subject may be determined to have respiratory syncytial virus if: the relative intensities of the biomarkers Granulocyte-Macrophage Colony-Stimulating Factor (GM-CSF), Pentraxin-3 (PTX-3), marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), or interleukin-27 (IL-27) are lower than the reference values and the relative intensity of interleukin-23 (IL-23) is higher than the reference values; and (a) one or more relative intensities of the biomarkers perforin (Perf), C-X-C motif chemokine ligand 11 (CXCL11), Epidermal Growth Factor (EGF), interleukin-8 (IL-8) and/or immunoregulatory alpha globulin (IRA) are lower than the reference values; (b) one or more of the relative intensities of the biomarkers interleukin-10 (IL-10), interleukin-21 (IL-21), and/or interleukin-2 (IL-2) are lower than the reference values; (c) one or more relative intensities Granzyme B (GRAND B), C-C motif chemokine ligand 24 (CCL24), interleukin-7 (IL-7), C-C chemokine ligand 20 (CCL20), interleukin-6 (IL-6), interleukin-4 (IL-4), interleukin-17 (IL-17), tumor necrosis factor alpha (TNFα), and/or interferon gamma (INFγ) are lower than the reference values; (d) one or more relative intensities of fractalkine (CX3CL1), C-C chemokine ligand 17 (CCL17), interleukin-15 (IL-15), interleukin-1β (IL-1β), and/or interleukin-12 (IL-12) are higher than the reference values; or (e) any combination of (a)-(d).

In further embodiments, the method further comprises recommending or initiating a suitable therapeutic measure, such as treatment for a subject. In some embodiments, a subject assessed to be at risk for or as having multisystem inflammatory syndrome in children, can be treated with one or more of: (1) immunomodulatory therapies such as, intravenous immunoglobulin (IVIG); (2) glucocorticoids, such as for example, methylprednisolone or prednisolone, to suppress inflammation; (3) biologic agents such as for example, anakinra (an IL-1 inhibitor), tocilizumab (an IL-6 inhibitor), or infliximab (a TNF-α inhibitor); (4) intravenous fluids to manage dehydration and support hydration; (5) antibiotics; (6) anticoagulants, such as aspirin or heparin to prevent thrombosis due to the hypercoagulable state associated with MIS-C; (7) mechanical ventilation or ECMO for severe respiratory or cardiac complications; (8) cardiac support, such as providing inotropes for cardiac dysfunction; or (9) any combinations of (1)-(8). In some embodiments, a subject assessed to be at risk for or as having Kawasaki disease, can be treated with one or more of: (1) immunomodulatory therapies such as intravenous immunoglobulin (IVIG); (2) aspirin to control fever and inflammation; (3) glucocorticoids, such as, for example, methylprednisolone or prednisolone, can be used if the subject does not respond to IVIG; (4) intravenous fluids to manage dehydration and support hydration; and (5) any combination of (1)-(4). For subjects assessed to be at risk for or as having both MIS-C and Kawasaki disease, a combination of the above therapeutic measures can be used to treat the subject. In still further embodiments, the method further includes monitoring a subject, such as a human pediatric subject. In some embodiments, a subject at risk for or suffering from MIS-C can be monitored for fever, multiorgan involvement, and laboratory evidence of inflammation. In addition, diagnostic and monitoring tests can also be used. For example, imaging tests, such as echocardiograms, chest X-rays, abdominal ultrasounds, and CT scans, can be used to evaluate organ involvement, particularly cardiac function. Laboratory tests such as the regular monitoring of inflammatory markers (e.g., CRP, ESR), cardiac enzymes (e.g., troponin, BNP), liver enzymes, and blood cell counts can also be used. In other embodiments, a subject at risk for or suffering from Kawasaki disease, can be monitored for laboratory evidence of inflammation. In addition, diagnostic and monitoring tests can also be used. For example, imaging tests, such as echocardiograms and electrocardiograms be used to evaluate organ involvement, particularly cardiac function. Laboratory tests such as regularly monitoring inflammatory markers (e.g., CRP, ESR), cardiac enzymes (e.g., troponin, BNP), liver enzymes, and blood cell counts can also be used.

In other embodiments, a subject assessed to be at risk for or suffering from SARS-CoV-2, can be treated with one or more of: (a) one or more antiviral medications (such as, Nirmatrelvir plus ritonavir (Paxlovid)), Remdesivir (Veklury), Molnupiravir (Lagevrio)); (b) one or more monoclonal antibodies (e.g., such as Sotrovimab (Xevudy), Vilobelimab (Gohibic)); (c) one or more immune modulators (e.g., such as, Baricitinib (Olumiant), Tocilizumab (Actemra), Anakinra (Kineret)); (d) Dexamethasone; (e) convalescent plasma; (f) rest; or (g) any combinations of (a)-(e).

In other embodiments, a subject assessed to be at risk for or suffering from respiratory syncytial virus (RSV), can be treated with one or more of: (a) over-the-counter fever reducers and pain relievers; (b) hydration therapy (e.g., maintaining hydration); (c) rest; (d) one or more antiviral medications (e.g., Ribavirin (Virazole)); (e) one or more monoclonal antibodies (e.g., Palivizumab (Synagis), Nirsevimab (Beyfortus)); (f) one or more vaccines (e.g., Arexvy (GSK), Abrysvo (Pfizer), mRESVIA (Moderna)); or (g) any combination of (a)-(f).

In further embodiments, the methods described herein are computer-implemented methods for assessing whether a subject is suspected of having or has multisystem inflammatory syndrome in children, Kawasaki disease, SARS-CoV-2, respiratory syncytial virus, an adenovirus, influenza A, B, C, or D, or parainfluenza that is carried out in an automated fashion on a data processing unit, such as a computer or similar data processing device. In some aspects, the method involves receiving a relative intensity value calculated on the amount or concentration of each of a plurality of first target biomarkers or first target biomarkers and second target biomarkers determined in a sample obtained from a subject. In some aspects, the subject is a pediatric subject. Once the intensity values are received for each of the plurality of first target biomarkers or first target biomarkers and second target biomarkers these relative intensity values are compared to reference values or reference value ranges for each of the first target biomarkers or first target biomarkers and second target biomarkers. Once this comparison is completed, the subject is assessed by on the comparison as discussed previously herein.

For the computer-implemented methods described herein, the present disclosure further contemplates a computer program, computer program product, or computer-readable storage medium having tangibly embedded the computer program, in which the computer program comprises instructions, which, when run on a data processing device or computer, carry out the methods described herein. Additionally, such computer-implemented methods can further comprise: (1) a computer or computer network comprising at least one processor, in which the processor is adapted to perform the method according to any of the embodiments described herein; (2) a computer loadable data structure that is adapted to perform the method according to any of the embodiments or aspects described herein while the data structure is being executed on a computer (3) a computer script, wherein the computer program is adapted to perform the method according to any of the embodiments or aspects described herein while the program is being executed on a computer; (4) a computer program comprising program means for performing the method according to any of the embodiments or aspects described herein while the computer program is being executed on a computer or on a computer network; (5) a computer program comprising program means as described herein, in which the program means are stored on a storage medium readable to a computer; (6) a storage medium, in which a data structure is stored on the storage medium and wherein the data structure is adapted to perform the method according to any of the embodiments or aspects described herein after having been loaded into a main and/or working storage of a computer or of a computer network; (7) a computer program product having program code means, wherein the program code means can be stored or are stored on a storage medium, for performing the method according to any of the embodiments or aspects described herein if the program code means are executed on a computer or on a computer network; (8) a data stream signal, typically encrypted, comprising data for amounts or values for each of the first target biomarkers or first target biomarkers and second target biomarkers; (9) a data stream signal, which can be encrypted, comprising the assessment provided by the methods described herein; and (10) any combination of (1)-(9).

### IV. Devices

In another embodiment, the present disclosure relates to devices for assessing a subject suspected of having or having multisystem inflammatory syndrome in children, Kawasaki disease, SARS-CoV-2, respiratory syncytial virus, an adenovirus, influenza A, B, C, or D, or parainfluenza. The devices of the present disclosure can comprise a measuring or analyzing unit for determining an amount of each of a plurality of (1) first target biomarkers in a sample; or (2) first target biomarkers and second target biomarkers in a sample. The measuring can also comprise a detection system for the first target biomarkers or the first target biomarkers and second target biomarkers. The first target biomarkers or first target biomarkers and second target biomarkers can be any of the target biomarkers described previously herein in Sections II (“Biomarker Panels”) and III (“Methods of Assessing Subjects Suspected of having Multisystem inflammatory syndrome in children, Kawasaki Disease, SARS-CoV-2, Respiratory Syncytial Virus, an Adenovirus, Influenza A, B, C, or D, and/or Parainfluenza”). In addition to determining the amount or concentration of each of the first target biomarkers or the first target biomarkers and second target biomarkers in the sample, the measuring unit also calculates or determines a relative intensity of each of the first target biomarkers or first target biomarkers and second target biomarkers based on the amount or concentration of each first target biomarker or first target biomarker and second biomarker measured or determined in the sample.

In addition to the measuring unit, the device also contains an evaluation unit, which is operably linked to the measuring unit. The evaluation unit comprises a database with stored references values or reference value ranges for each of the target biomarkers and a data processor that contains instructions for carrying out a comparison of the calculated relative intensity of each first target biomarker or each first target biomarker and each second target biomarker with the reference values or reference value ranges. The evaluation unit can automatically receive the reference values or reference value ranges for the target biomarkers (e.g., the first target biomarkers or first target biomarkers and second target biomarkers) from the measuring unit.

As used herein, the term “device” refers to a system comprising a measuring or analyzing unit and an evaluation unit operatively linked to each other to allows for the determination of the amounts or concentration of target biomarkers (e.g., first target biomarkers or first target biomarkers and second target biomarkers) and evaluation thereof according to the methods described herein such that an assessment can be provided. In an aspect, the device includes at least one detection agent disclosed herein that specifically binds to its corresponding target biomarker is used as a capture reagent or capture-specific binding partner, wherein the capture reagent is immobilized on a solid support. In an embodiment, the solid support is a particle, microparticle, bead, plate, well, chip, or any combination thereof. In an embodiment, the solid support is a chip.

The measuring or analyzing unit comprises at least one reaction zone containing a plurality of biomarker detection agents for each target biomarker as described previously. In some aspects, each of the biomarker detection agents is immobilized on solid support (e.g., such as a particle, microparticle, bead, plate, well, or chip) or carrier, which subsequently contacts the sample. In some aspects, the reaction zone contains a biomarker panel, such as a GCFP biosensor chip containing the plurality of biomarker detection agents, Additionally, in the reaction zone, conditions can be applied that allow for the specific binding of each biomarker detection agent to their corresponding target biomarker in the sample.

The reaction zone may either allow for sample application or be connected to a loading zone where the sample is applied. In the latter case, the sample can be actively or passively transported via the connection between the loading zone and the reaction zone to the reaction zone. Moreover, the reaction or zone can also connected to a detector. Any detector can be used. In some aspects, the detector is a GCFP reader (Ciencia, Inc., CT, USA). The connection shall be such that the detector can detect the binding of the target biomarkers to their biomarker detection agents. Suitable connections depend on the techniques used to measure the target biomarkers' presence or amount. For example, for optical detection, transmission of light may be required between the detector and the reaction zone. In contrast, for electrochemical determination, a fluidal connection may be required, e.g., between the reaction zone and an electrode. The detector shall be adapted to detect the determination of the amount of the biomarkers. The determined amount can be subsequently transmitted to the evaluation unit. The evaluation unit comprises a data processing element, such as a computer, with an implemented algorithm for determining the amount present in the sample.

The processing unit referred to herein can comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like. A data processing element may be a general-purpose computer or a portable computing device. Multiple computing devices can also be used together, such as over a network or other methods of transferring data, for performing one or more steps of the methods disclosed herein. Exemplary computing devices include desktop computers, laptop computers, personal data assistants (“PDA”), cellular devices, smart or mobile devices, tablet computers, servers, and any combination thereof. In general, a data processing element comprises a processor capable of executing a plurality of instructions (such as a program of software).

The evaluation unit comprises or has access to a memory. Memory is a computer-readable medium and may comprise a single storage device or multiple storage devices, located either locally with the computing device or accessible to the computing device across a network, for example. Computer-readable media may be any available media that the computing device can access and includes both volatile and non-volatile media. Further, computer-readable media may be one or both removable and non-removable media. By way of example, and not limitation, computer-readable media may comprise computer storage media. Examples of computer storage media include but is not limited to, RAM, ROM, EEPROM, flash memory or any other memory technology, CD-ROM, Digital Versatile Disk (DVD) or other optical disk storage, magnetic cassettes, magnetic tape, magnetic disk storage or other magnetic storage devices, or any other medium which can be used for storing a plurality of instructions capable of being accessed by the computing device and executed by the processor of the computing device.

In additional embodiments, the software may include instructions which, when executed by a processor of the computing device, may perform one or more steps of the methods disclosed herein. Some of the instructions may be adapted to produce signals that control operation of other machines and thus may operate through those control signals to transform materials far removed from the computer itself. These descriptions and representations are the means used by those skilled in the art of data processing, for example, to most effectively convey the substance of their work to others skilled in the art.

The plurality of instructions may also comprise an algorithm which is generally conceived to be a self-consistent sequence of steps leading to a desired result. These steps require physical manipulations of physical quantities. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as values, characters, display data, numbers, or the like as a reference to the physical items or manifestations in which such signals are embodied or expressed. It should be borne in mind, however, that all of these and similar terms are to be associated with the appropriate physical quantities and are merely used here as convenient labels applied to these quantities.

The evaluation unit may also comprise or have access to an output device. Exemplary output devices include fax machines, displays, printers, and files. In some embodiments, a computing device may perform one or more steps of a method disclosed herein and thereafter provide an output via an output device relating to a result, indication, value, or other factor of the method.

### V. Kits

In another embodiment, the present disclosure relates to kits for assessing a subject suspected of having or having multisystem inflammatory syndrome in children, Kawasaki disease, SARS-CoV-2, respiratory syncytial virus, an adenovirus, influenza A, B, C, or D, and/or parainfluenza. In some aspects, the kit can comprise instructions for carrying out the methods and assessments described in Section III (“Methods of Assessing Subjects Suspected of having Multisystem inflammatory syndrome in children, Kawasaki Disease, SARS-CoV-2, Respiratory Syncytial Virus, an Adenovirus, Influenza A, B, C, or D, and/or Parainfluenza”) herein. Instructions included in kits can be affixed to packaging material or included as a package insert. While the instructions are typically written or printed materials, they are not limited to such. Any medium capable of storing such instructions and communicating them to an end user is contemplated by this disclosure. Such media include but are not limited to, electronic storage media (e.g., magnetic discs, tapes, cartridges, chips), optical media (e.g., CD ROM), and the like. As used herein, the term “instructions” can include the address of an internet site that provides the instructions.

In some aspects, the kit can comprise any of the biomarker panels described in Section II (“Biomarker Panels”) herein. In some aspects, the biomarker panel is provided in the kit as a chip, such as a grating-coupled fluorescent plasmonic biosensor chip.

In other aspects, the kit can comprise a plurality of first biomarker detection agents that specifically binds to its corresponding target biomarker (e.g., first target biomarker or a first target biomarker and a second target biomarker). In some aspects the plurality of first biomarker detection agents are one or more isolated antibodies. In other aspects, the plurality of first biomarker detection agents is one or more isolated aptamers. One or more solid supports can be included in the kit if the first biomarker detection agent is to be used as a capture reagent or capture specific binding partner. Alternatively, one or more detectable labels can be included in the kit if the first biomarker detection agent is to be used as a detection reagent or a detection specific binding partner. In other embodiments, the kit comprises a plurality of first biomarker detection agents and a plurality of second biomarker detection agents. In some aspects, one or more detectable labels is provided in the kit where either the first biomarker detection agent, the second biomarker detection agent or both the first biomarker detection agent and the second biomarker detection agent are labeled with one or more detectable labels. Moreover, in some aspects, the second biomarker detection agent can be one or more isolated antibodies or one or more isolated aptamers.

Alternatively or additionally, the kit can comprise a calibrator or control, and/or at least one container (e.g., tube, microtiter plates or strips) for conducting the methods described, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the methods described in Section III (“Methods of Assessing Subjects Suspected of having Multisystem inflammatory syndrome in children, Kawasaki Disease, SARS-CoV-2, Respiratory Syncytial Virus, an Adenovirus, Influenza A, B, C, or D, and/or Parainfluenza”). Moreover, the instructions also can include instructions for generating a reference value or reference value range.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish method or assay performance characteristics, and further optionally are useful indicators of the integrity of the immunoassay kit reagents, and the standardization of methods and assays.

The kit can also optionally include other reagents required to conduct the methods described herein or facilitate quality control evaluations, such as buffers, salts, enzymes, enzyme co-factors, substrates, detection reagents, and the like. Other components, such as buffers and solutions for the isolation and/or treatment of a sample (e.g., pretreatment reagents), also can be included in the kit. The kit can additionally include one or more other controls. One or more of the kit's components can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for saliva, whole blood, plasma, or serum sample). Where appropriate, the kit can also contain reaction vessels, mixing vessels, and other components that facilitate the preparation of reagents or the test sample. The kit can also include one or more instruments for assisting with obtaining a sample, such as a syringe, pipette, forceps, measured spoon, or the like.

The present disclosure is illustrated and further described in more detail with reference to the following non-limiting examples. Section headings as used in this section and the entire disclosure herein are merely for organizational purposes and are not intended to be limiting.

### EXAMPLES

### Methods and Materials

### Biological Samples

The prospective observational study included 260 subjects, birth to <21 years of age, enrolled Apr. 1, 2020, through May 1, 2022, at sites in Connecticut, United States and Cali, Colombia. After obtaining Institutional Review Board (IRB) approval (#21-004), subjects were enrolled in two cohorts: an experimental cohort (Cohort A) and a reference cohort (Cohort B). The experimental cohort was comprised of subjects who were positive for SARS-CoV-2 infection per antigen or Polymerase Chain Reaction (PCR) testing and hospitalized for COVID-19 symptoms (subgroup A1); hospitalized and meeting the Center for Disease Control's criteria for MIS-C (subgroup A2); and non-hospitalized subjects testing positive for SARS-CoV-2 by antigen or PCR, (subgroup A3). The reference cohort was comprised of SARS-CoV-2 negative subjects who were: hospitalized with a diagnosis of Kawasaki disease (subgroup B1); hospitalized because of an acute respiratory viral infection (subgroup B2); or healthy controls undergoing routine ambulatory surgery (subgroup B3). To ensure correct subgroup assignment, cases were independently reviewed and adjudicated by three pediatric specialists. Demographic data, health history, current symptoms, and in-patient treatments and diagnostic testing results were collected at baseline. Subjects will be followed for up to four years via survey for health and SARS-CoV-2 vaccine status.

### Patient Saliva Processing

Patient saliva samples were stored at −80° C. for at least 24 hours prior to processing. The samples were placed in the biosafety cabinet and allowed to thaw at room temperature for approximately 20 minutes. Once thawed, the exterior of sample tubes were cleaned with alcohol wipes and centrifuged at 200×g for 15 mins at 4° C. For samples to be analyzed with the grating-coupled fluorescent plasmonic biosensor chip, 500 μL of supernatant was aliquoted and 35 μL of protease inhibitor dissolved in 1 mL Phosphate Buffered Saline (PBS) was added (Piercer™ protease inhibitor tablet, Thermo Fisher Scientific, MA, USA) was added. For samples intended to be used in 16S RNA PacBio sequencing, 250 μL of supernatant was aliquoted and heat-inactivated for 30 mins at 56° C. Once processed, samples were stored at −80° C. until use.

### Patient Serum Processing

Sera were isolated from patient blood samples by resting the blood for approximately 30 minutes after collection to allow for coagulation. The non-coagulated fraction was transferred into a 15 mL tube and centrifuged at 1000×g for 15 minutes. Supernatant was then transferred to a new tube and centrifuged at 1000×g for 5 minutes. Supernatants were aliquoted into 200 μL cryovials and stored at −80° C. until transferred to participating laboratories.

### Matched Pair Enzyme-Linked Immunoassay (ELISA) Reagent Validation

Antibody validation was performed by ELISA. Capture antibody (R&D Systems Inc, MN, USA; Mabtech AB, Nacka Strand, Sweden; CusaBio Technology LLC, TX, USA; Bio-Rad Laboratories, CA, USA) was diluted in PBS and 100 μL/well was used to coat wells of an Immulon II HB flat bottom micrometer plate (Thermo Fisher Scientific, MA, USA). After incubation at room temperature overnight, the plate was aspirated and washed with PBST (160 g NaCl, 4 g KCl, 4 g Kh₂PO₄, 23 g NaHPO₄, 5 mL Tween 20, 4 g NaN₃ dissolved into 20 L H₂O) using an ELx405 automated plate washer (BioTek), then blocked with 250 μL of 2% Bovine Serum Albumin (BSA) in PBS for 1.5 h at room temperature (RT). The plate was washed and incubated with 100 μL of diluted recombinant cytokine in reagent diluent (1% bovine serum albumin in PBS) or PBS, according to kit instructions, for 2 h at RT. Following this incubation, the plate was washed again and incubated with 100 μL/well of manufacturer's recommended concentration of secondary, biotinylated, antibody for 2 h at RT. After washing, 100 μL/well of recommended concentration of streptavidin-HRP was added to wells and incubated for 20 minutes at RT. After a final wash, 100 μL/well of a 1:1 mixture of color reagent A (H₂O₂) and color reagent B (TMB (3,3′,5,5′-Tetramethylbenzidine) substrate was added to the plate and incubated for 20 minutes at RT in the dark. 50 μL of 2N H₂SO₄ was added to stop the reaction and color production was measured at 570-450 nm in a Spectramax i3x plate reader (Molecular Devices, CA, USA).

### Printing of Grating-Coupled Fluorescent Plasmonic (GCFP) Biosensor Chip

Biosensor chips were fabricated and processed as described previously with the following modifications (Chou et al., 2020, “A Fluorescent Plasmonic Biochip Assay for Multiplex Screening of Diagnostic Serum Antibody Targets in Human Lyme Disease.” PLOS ONE 15 (2): e0228772. https://doi.org/10.1371/JOURNAL.PONE.0228772; Cady et al., 2021, “Multiplexed Detection and Quantification of Human Antibody Response to COVID-19 Infection Using a Plasmon Enhanced Biosensor Platform.” Biosensors & Bioelectronics 171 (January): 112679. https://doi.org/10.1016/J.BIOS.2020.112679; Rice et al., 2012, “Antigen-Specific T Cell Phenotyping Microarrays Using Grating Coupled Surface Plasmon Resonance Imaging and Surface Plasmon Coupled Emission.” Biosensors & Bioelectronics 31 (1): 264. https://doi.org/10.1016/J.BIOS.2011.10.029; Molony et al., 2012, “Mining the Salivary Proteome with Grating-Coupled Surface Plasmon Resonance Imaging and Surface Plasmon Coupled Emission Microarrays.” Current Protocols in Toxicology Chapter 18 (SUPPL.53), https://doi.org/10.1002/0471140856.TX1816S53). Captured antibodies were diluted to 250 μg/mL with PBS (Table 1) and printed on the GCFP chips using a 0.35 mm diameter microarray pin and a SpotBot II microarray printer (Arraylt, CA, USA) or an XactII microarray printer (LabNEXT, NJ, USA). Each capture antibody was spotted to create five individual regions of interest (ROI) per analyte on each biosensor chip. During printing, microarrays were kept at a relative humidity of 70% and at ambient temperature (AT) (˜25° C.). After printing, chips were allowed to dry at a relative humidity of 70% and AT (˜25° C.) for 1 hr. Chips were then transferred to a 50 mL polypropylene tube containing desiccant and stored for up to 4 weeks, with no significant signal loss (data not shown).

TABLE 1
Regions of interest used on first generation chip

| Marker class | Analytes |
|---|---|
| Cytokines | IL-7, IL-21, IFNg. IL-15, IL-6, |
| | IL-10, IL-17, IL-2, IL-4, IL-1β, |
| | TNFα, IL-33, IL-18 (total), |
| | IL-18 BPa |
| Chemokines | CCL20, CXCL11, CCL24, CCL2, |
| | CCL3, CCL7, CXCL10, CXCL9, |
| | CCL5, CCL17, CXCL8, CX3CL1 |
| Interleukin receptor | sCD25 |
| Kidney function | Cystatin C |
| Indicator of Inflammation | CRP |
| Range of biological processes | Galectin-3 |
| Infection indicator | Procalcitonin |
| Type 1 membrane protein | B7-1/CD80 |
| Protein found on antigen | Perforin |
| presenting cells | |
| Peptide involved in central and | Neuropeptide Y |
| peripheral nervous systems | |
| Serine protease that is expressed | Marapsin/Pancreasin |
| strongly in the pancreas | |
| Cell death | LDH(A) |
| Protein fragments produced | D-Dimer |
| when a blood clot | |
| Tissue damage | Cardiotrophin-1 |
| Spike protein receptor | ACE-2 |
| Heat shock protein | HSP70 |
| Iron levels | Ferritin |

### GCFP Detection Assay

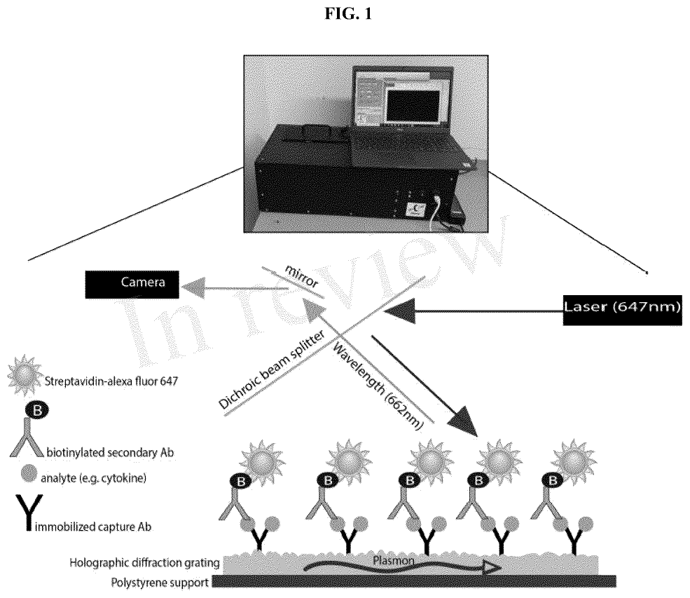

Biosensor chips were processed as previously described with the following modifications (Chou et al., 2020; Molony et al., 2012; Rice et al., 2012) (FIG. 1). Chips were fitted with plexiglass windows and gaskets to allow fluid to pass over the surface of the chip. The chip was then blocked with 80 μL of Superblock™ T20 (PBS) blocking buffer (Thermo Fisher Scientific, MA, USA) in a static incubation for 1 hour. Each chip was then washed with PBS-T at a flow rate of 0.5 mL/min for 3 minutes using a peristaltic pump. PBS doped with recombinant protein (20 ng/mL) or patient samples (80 μL of serum or saliva samples diluted with PBS to 1.5 mL) were then recirculated over the chip for 90 minutes at 0.5 mL/min for 3 minutes. Each chip was then washed with PBS-T at 0.5 mL/min for 3 minutes. Biotinylated secondary antibody mixtures were created with all detection antibodies at a concentration of 200 ng/mL and then recirculated over the chip for 90 minutes. The chip was then washed with PBS-T at 0.5 mL/min for 3 minutes and then PBS containing 500 ng/mL of streptavidin-AlexaFluor 647 (Thermo Fisher) was recirculated over the chip for 1 hour. The chip was then washed for a final time with PBS-T at 0.5 mL/min for 3 minutes and 70 μL of PBS was injected into the flow cell. ROIs were then identified using Ciencia software, Enhanced fluorescence Reader V2.3, and fluorescence intensity for each ROI was collected using a GCFP reader (Ciencia, Inc.). A GCFP detection ratio was used to normalize each ROI value as previously described (Cady et al., 2021).

16S-23S rRNA Microbiome Sequencing

DNA was extracted using the Complete Lyse & Purify kit (Shoreline Biome, Farmington, CT, USA). Saliva samples were thawed on ice, and between 50 μL and 200 μL of saliva was pelleted by centrifuged at 5500 rpm for 10 minutes. Pellets were resuspended in nuclease-free water, then purified according to the manufacturer's protocol. Extracted DNA was amplified with the StrainID Amplify kit according to manufacturer protocol (PacBio Complete StrainID Kit Instruction https://directus.biocat.com/uploads/biocat/originals/shoreline-wave-for-pacbio-technical-manual-v01.pdf). 10 μL of PCR mix (Shoreline Biome, USA) were added to each well with barcoded primers and mixed by pipetting. The PCR reaction was carried out on a thermocycler (BioRad, Hercules, CA, USA) according to manufacturer protocol. Amplicons were screened on a QIAxcel (Qiagen, Germantown, MD, USA) Advanced system using the Fast Analysis protocol. Samples were then pooled together based on band intensity. Samples were cleaned using the GeneRead Size Selection Kit (Qiagen, USA) according to manufacturer protocol and resuspended in 50 μL of elution buffer. After verifying that both pools were pure, amplicons were pooled and sequenced on a PacBio Sequel IIe. Sequences were demultiplexed with SBAnalyzer. In order to use DADA2 software (https://benjjneb.github.io/dada2/) to call ASVs with pooling enabled, samples were split into groups of approximately 35 samples. Afterwards, results for all samples were merged together, and taxonomy was assigned using the Athena database. Data analysis was performed in QIIME2 and R. Taxa that are different in relative abundance between cohorts were identified by performing pairwise comparisons with ANCOM in QIIME 2. Visualizations were made with GraphPad Prism and microViz. The code used is available at https://github.com/brandon-osullivan/Code-for-Maltz-Matyschsyk-et-al-2022.

### Microsphere Immunoassay (MIA) for Cytokines Identification

Cytokines/Chemokine levels in the serum samples were measured in duplicate using the Luminex®200™ instrument and Milliplex®MAP kits from EMD Millipore (Cat #HSTCMAG-28SK, HCYTA-60K, HCYP2MAG-62K and HCYP4MAG-64K) according to manufacturer's protocol. A 96 well plate provided with the kit was first washed with 200 μl per well of wash buffer. The wash buffer was then discarded, and 25 μl of serum sample was added to the plate in duplicate along with 50 μl of standards and controls as provided with each kit. Assay buffer (25 μl) was then added to the sample wells followed by 25 μl of premixed magnetic beads (provided with the kit) to each well. The plate was covered with a dark lid and placed on a plate shaker (200 rpm on a Barnstead 4625 Titer plate shaker) overnight at 4° C. in a dark room. On the following day, the plate was washed 3× with 200 μl of wash buffer using BioTek ELx405′ microplate washer with magnetic capture: after washing, detection antibodies (50 μl) were added to each well and the plate was incubated for 1 hour on a plate shaker covered with a foil. Streptavidin-Phycoerythrin (50 μl) was added to each well and the plate was incubated for 30 min on a plate shaker covered with foil. Finally, the plate was washed 3× with 200 μl of wash buffer using BioTek ELx405™ microplate washer and analyzed using the Luminex® 200™ instrument (Calibrated each week with Luminex 200 Calibration and Performance Verification kits: Cat #LX2R-CAL-K25, LX2R-PVER-K25) with 150 μl of Sheath Fluid present in each well. Standard curves were generated using the Luminex xPONENT® software and the concentrations of cytokines/chemokines in the serum samples were calculated using these standard curves in pg/mL.

### MIA for Ig Response to Sars-CoV-2 Epitopes

Specimens were assessed for the presence of antibodies reactive with SARS-CoV-2 using an MIA. Briefly, recombinant SARS-CoV-2 N protein antigen (Native Antigen Company, UK) and the receptor binding domain (RBD) of the SAR-CoV-2 spike protein (MassBiologics, MA, USA) were covalently linked to the surface of fluorescent microspheres (Luminex Corporation, TX, USA). Additional target antigens (S1 or S2 domain (Native Antigen, UK) of SARS-CoV-2 were included in the multiplexed microsphere assay. Serum samples (25 μl) and antigen-conjugated microspheres (25 μl) were mixed and incubated before washing and further incubation with phycoerythrin-conjugated antisera. The antisera used were chosen to specifically recognize, as indicated, total antibodies (pan-Ig), or, individually IgM, IgA, IgG, IgG1, IgG2, IgG3, IgG4. All detecting antibodies were purchased from Southern Biotech (AL, USA). After washing, the median fluorescence intensity (MFI) was quantified with a FlexMap 3D Luminex analyzer (Luminex Corporation, TX, USA). Results were either direct MFI values with reactivity based upon a defined cutoff MFI, or, were normalized by comparison to the MFI of the negative controls and expressed as the ratio between the two (P/N). Positive reactivity is determined by a result that is ≥6 SD above the cutoff; results that fall between 3 SD and 6 SD are considered “Indeterminate.”

Specimens were assessed for the presence of antibodies reactive with SARS-CoV-2 using a multisphere immunoassay (MIA). This assay utilizes polystyrene microspheres (Luminex Corporation, TX, USA) to which specific protein antigens are chemically attached (coupled). Because the microspheres can be purchased in different lots which each contain their own, individual fluorescence signature (i.e., the microspheres contain distinguishable fluorescent tags which provide signals at individually identifiable wavelengths), different protein-antigen-microsphere combinations may be included in the same tube to measure distinct antibody binding profiles. Those antibodies which are reactive, and therefore bind to, different proteins in the tube are detected using another distinctly fluorescent agent (phycoerythrin) which is attached to an anti-human immunoglobulin (Ig) serum. That anti-Ig can be modified to specifically detect different classes, termed “isotypes” of human immunoglobulin (e.g., IgM, IgG, IgA, IgG, IgG1, IgG2, IgG3, as desired). For the SARS-CoV-2 MIA, recombinant SARS-CoV-2 N protein antigen (Native Antigen Company, UK) and the receptor binding domain (RBD) of the SAR-CoV-2 spike protein (MassBiologics, MA, USA) were covalently linked to the surface of such fluorescent microspheres (Luminex Corporation, TX, USA). Additional target antigens (S1 or S2 domain (Native Antigen, UK) of SARS-CoV-2 were included in the multiplexed microsphere assay. A solution (25 μl) containing all of the target antigen microspheres was placed in a tube to which a serum sample (25 μl) was added and mixed. The tubes were then incubated for 30 minutes at 37° C. with constant shaking. The samples were then washed 3 times with phosphate-buffered saline (PBS), retaining the microspheres, which were then mixed with the detecting conjugate (PE-anti-IgG, etc.) (50p), followed by another 30 minute incubation at 37° C. with constant shaking. The microspheres were then washed again 3 times with PBS before resuspension in PBS (110 μl) and analysis using the FlexMap 3D Luminex analyzer (Luminex Corporation, TX, USA). Individual microsphere-antigen sets were identified by the analyzed and the amount of PE-conjugate associated with each individual microsphere-antigen set, measured as the median fluorescence intensity (MFI), was also quantified with the FlexMap 3D Luminex analyzer. Those MFIs reflected the presence and relative amounts of antibodies, total or of different Ig isotype, in a patient's serum, which could bind to individual SARS-CoV-2 antigens. Results from the test were expressed as either direct MFI values, with reactivity based upon a defined cutoff (mean MFI of a panel of 94 negative SARS-CoV-2 antibody specimens), or, were normalized by comparison to the MFI of the negative control, cutoff panel and expressed as the ratio (Index Value) between the two (P/N). By either method of expression, reactivity was determined by a result that was >6 SD above the cutoff; results that fell between 3 SD and 6 SD were considered to be “Indeterminate”. Results that fell below 3 SD above the cutoff were considered as non-reactive.

### Optimization of Biosensor Chip

The initial biosensor chip configuration was optimized by comparison to an established Luminex assay. First, a dose response curve was performed examining the limit of detection for the 11 analytes that were evaluated in both the GCFP assay and the Luminex data using parameters that had been employed with the first-generation GCFP chip. Chips were spotted as described above with the capture antibodies for the following analytes: TNF-α, IL-7, IL-6, IL-4, IL-21, IL-2, IL-1β, IL-17A, IFN-δ, CCL-3, CCL20. Recombinant proteins (R&D Systems, MN, USA; Shenandoah Biotechnology, Inc, PA, USA) were then diluted to 5 ng/mL, 1 ng/mL, 200 pg/mL, and 1 pg/mL and recirculated over the chip as described above. Detection ratios were calculated, and the limit of detection was determined for each matched pair set by GCFP. The capture and secondary antibodies were then optimized to reach values detectable with the Luminex MIA assay.

### Results

### Identification and Validation of Regions of Interest

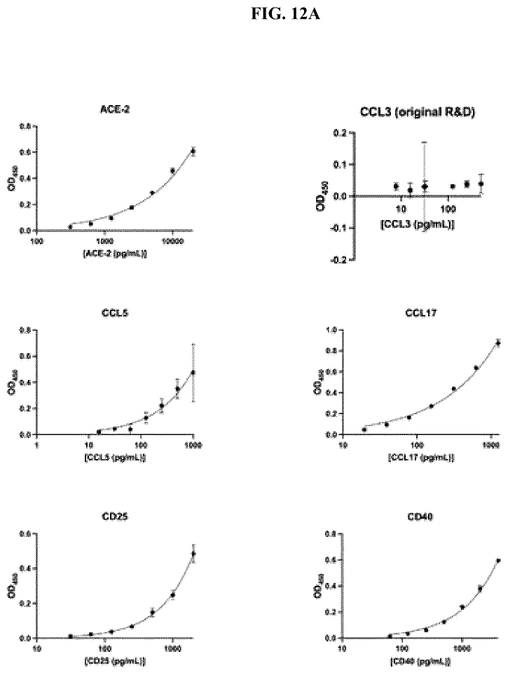

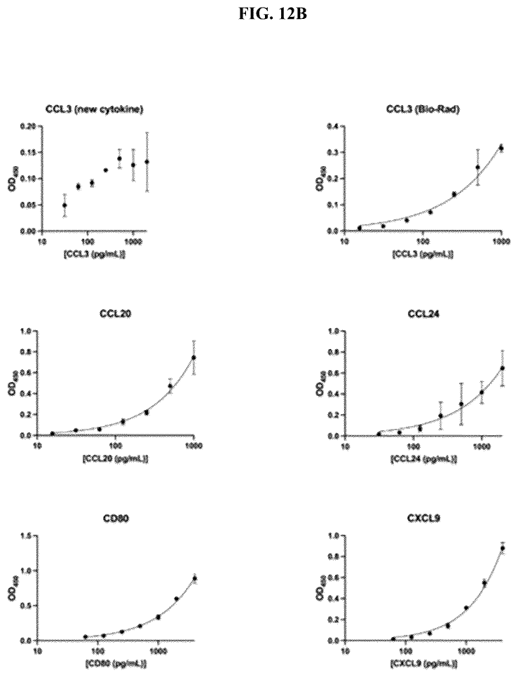

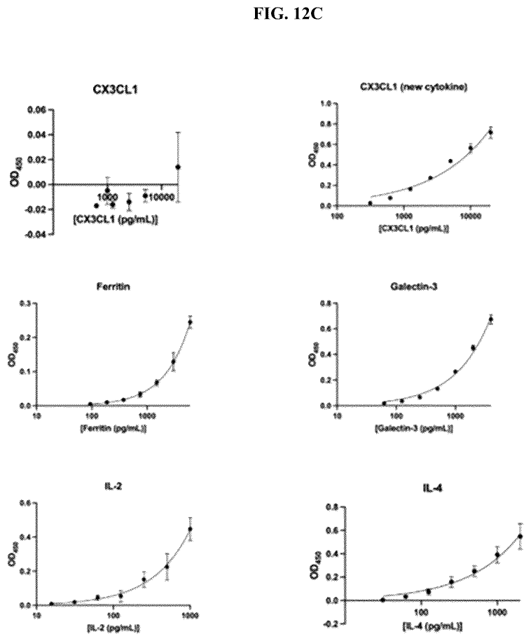

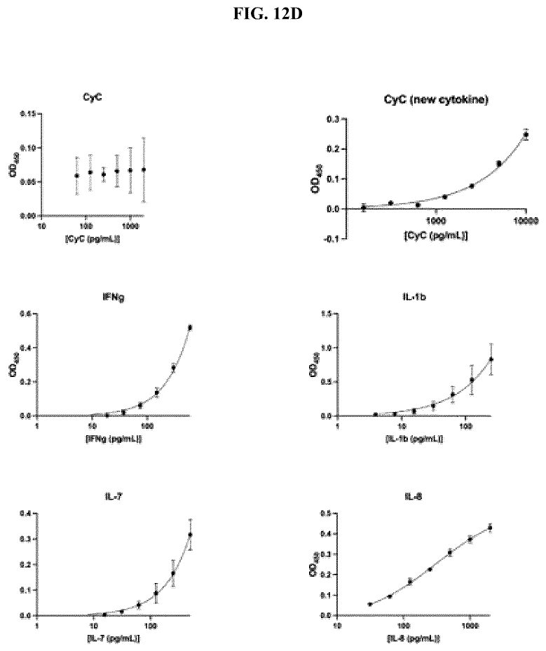

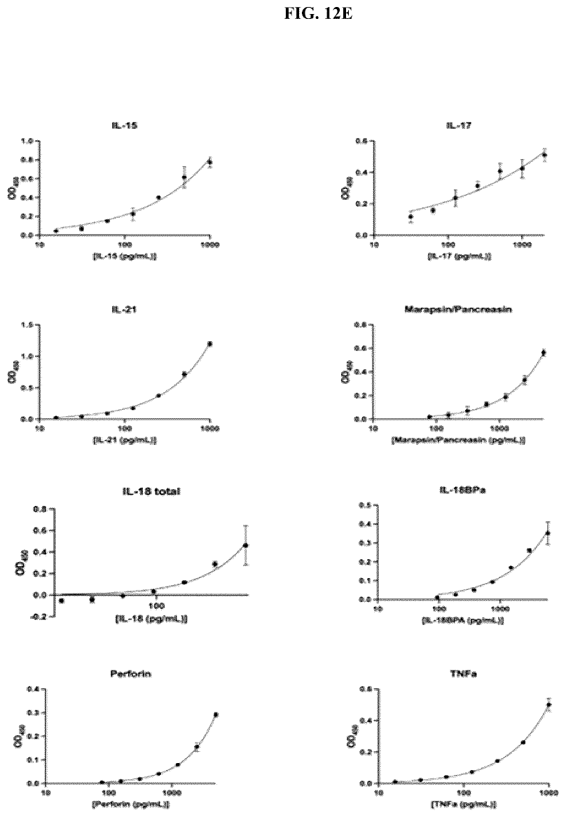

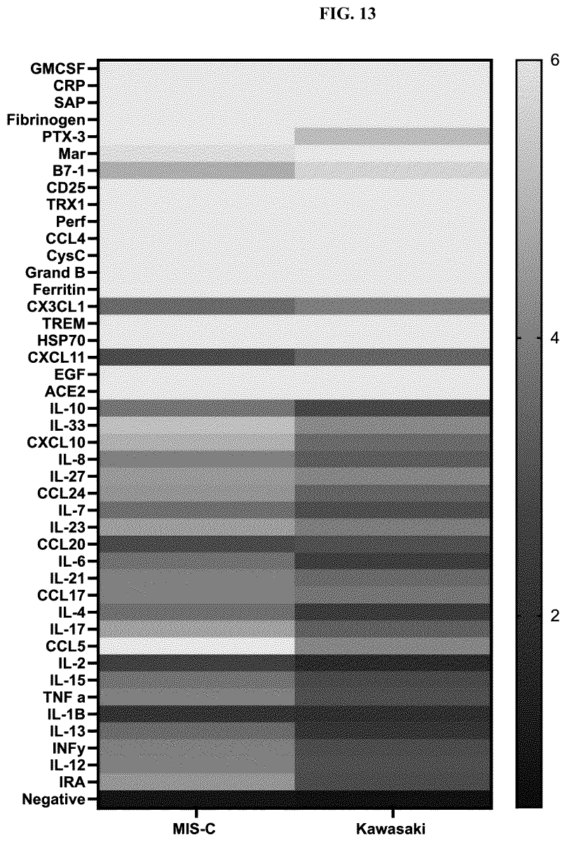

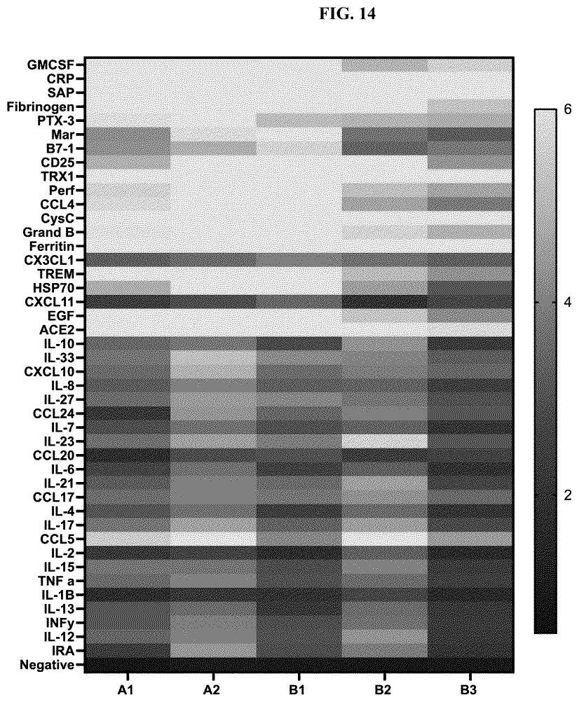

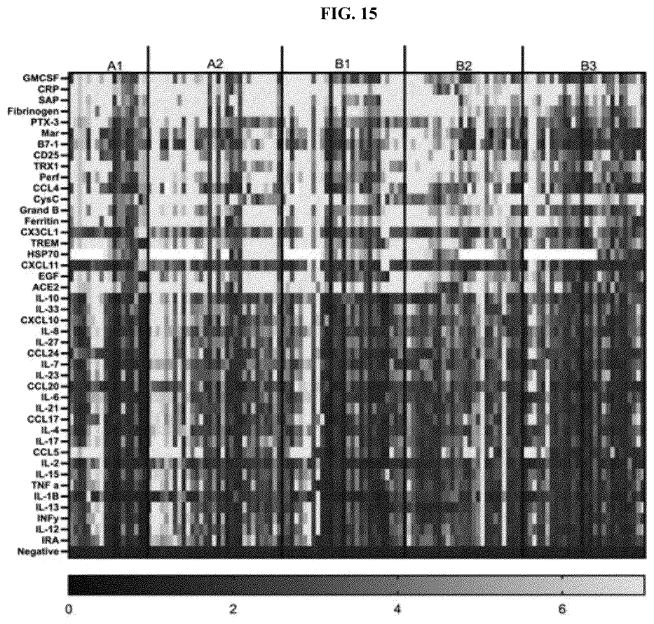

Since MIS-C and COVID-19 are relatively new disease states, a first generation GCFP biosensor chip was developed based on literature available early in the pandemic. These studies showed an array of cytokines, chemokines, tissue damage markers, and inflammatory indicators that differed significantly in children with MIS-C, COVID-19, and Kawasaki (Table 1). The goals with the first-generation chip were to: 1) identify commercially available matched pair antibodies for our ROIs to differentiate MIS-C from COVID-19, Kawasaki, respiratory viral infections, and healthy controls, and 2) develop a GCFP assay to simultaneously evaluate if these markers could be used as a disease-specific biosignature. 42 commercially available matched pair antibodies were validated by ELISA using recombinant proteins (FIGS. 12A-12E show representative validations and includes some assays where specific commercial reagent batches were not validated, e.g. CCL3, CyC, and CXCL1). Nine commercially available kits that did not meet our ELISA standards when performed following manufacture protocols, and were omitted from further analysis (data not shown).

### Development of the Grating-Coupled Fluorescent Plasmonic Biosensor Chip Assay

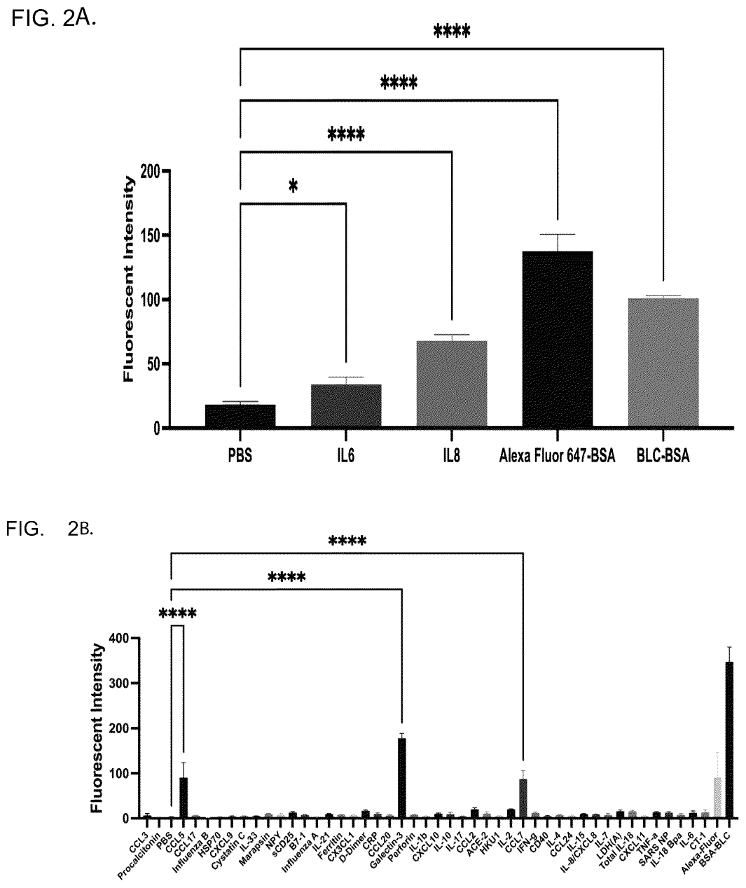

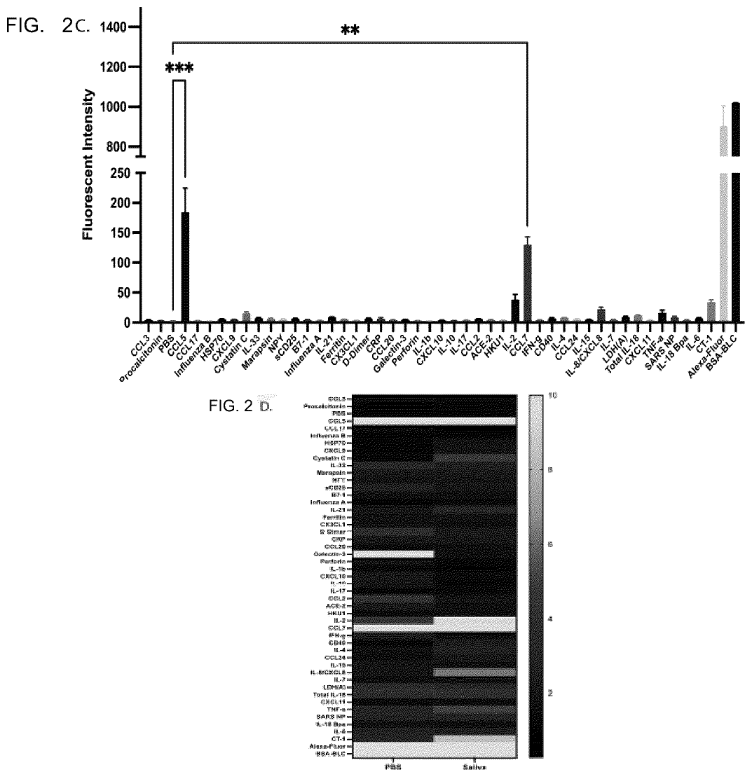

Once matched-pair kits were validated using ELISA, their ability to perform a sandwich-based immunoassay on the gold-coated nanoscale grating surface chip was then tested. IL-6, IL-8 and both positive (Alexafluor-647 and biotin-BSA) and negative (PBS) controls (FIG. 1 and FIG. 2A) are shown as examples. When compared to PBS (negative control), the fluorescent intensity within the ROIs for IL-6, IL-8 and positive controls was significantly higher, indicating 20 ng/mL of IL-6 and IL-8 recombinant proteins could be detected with GCFP technology. Next, the specificity of the ROIs was examined by spotting all the capture antibodies in Table 1 onto the chip and recirculating 20 ng/mL of recombinant CCL5, galactin-3 and CCL7 diluted in PBS over the biosensor chip (FIG. 2B). When compared to PBS (negative control ROIs), the fluorescent intensity within the ROIs for CCL5, galactin-3 and CCL7 were significantly higher, indicating that the GCFP assay was able to capture and detect these recombinant proteins at cognate ROIs and that there was no significant cross-reactivity between these recombinant proteins and unrelated ROIs. To evaluate the use of human saliva as the sample matrix, CCL5 and CLL7 (20 ng/mL) were spiked into commercially available pooled human saliva (Innovative Research, MI, USA) (FIG. 2C). Spiked samples in pooled saliva matrix produced higher values than when the same analytes were diluted in PBS owing to the presence of endogenous CCL5 and CCL7 in the saliva pool. A heat map was created using the detection ratio for each set of 5 ROIs (FIG. 2D). The heat map shows the detection ratio and indicates positive detection of all recombinant proteins when normalizing to the negative control ROIs on the same chip.

### Analysis of Patient Samples by GCFP

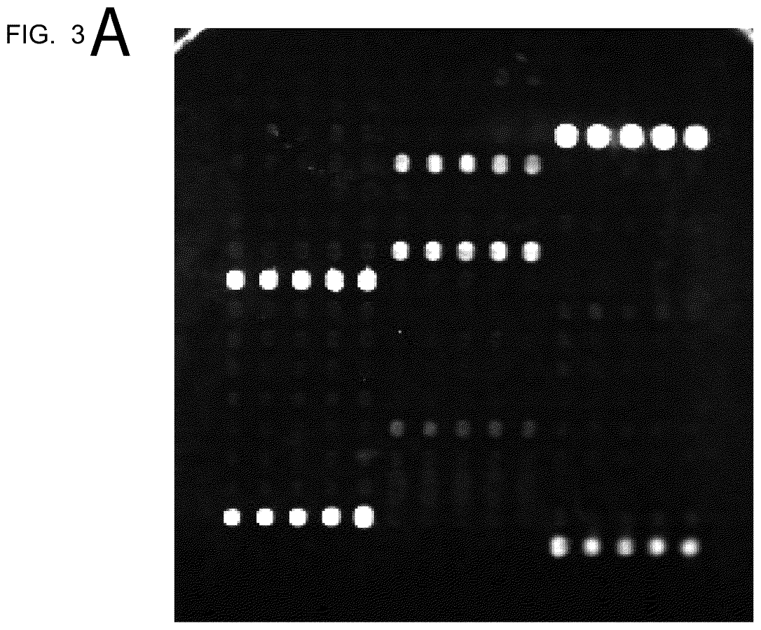

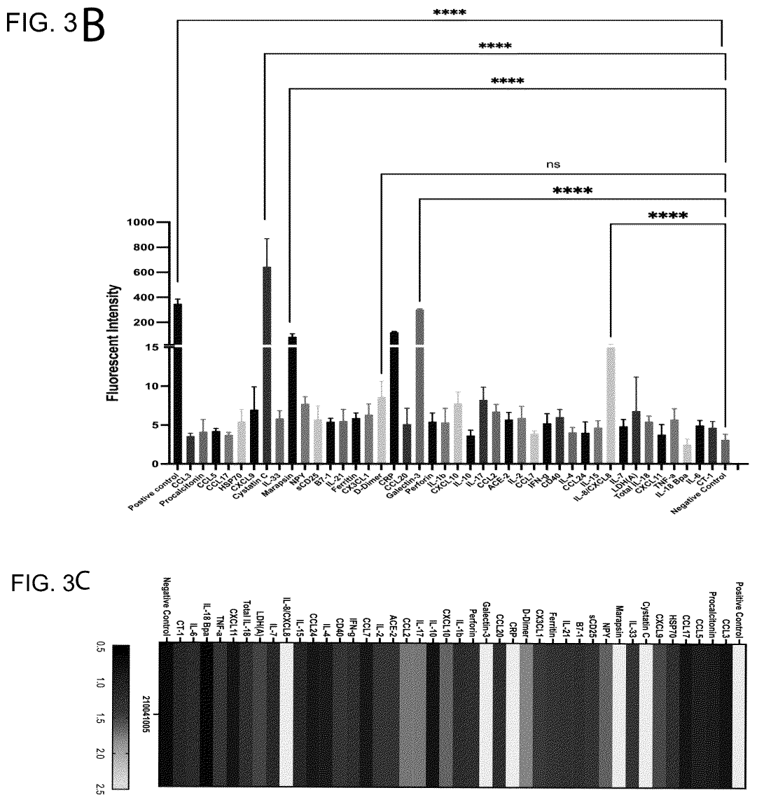

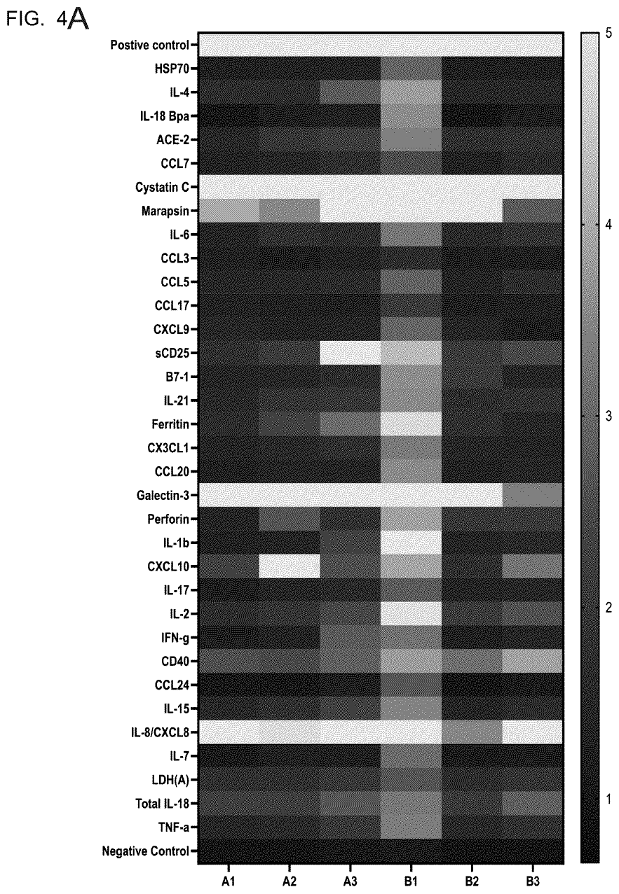

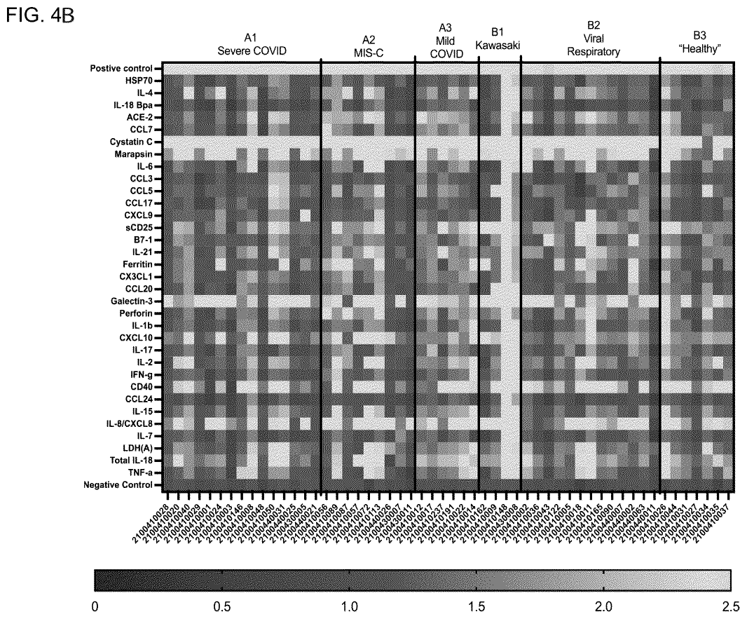

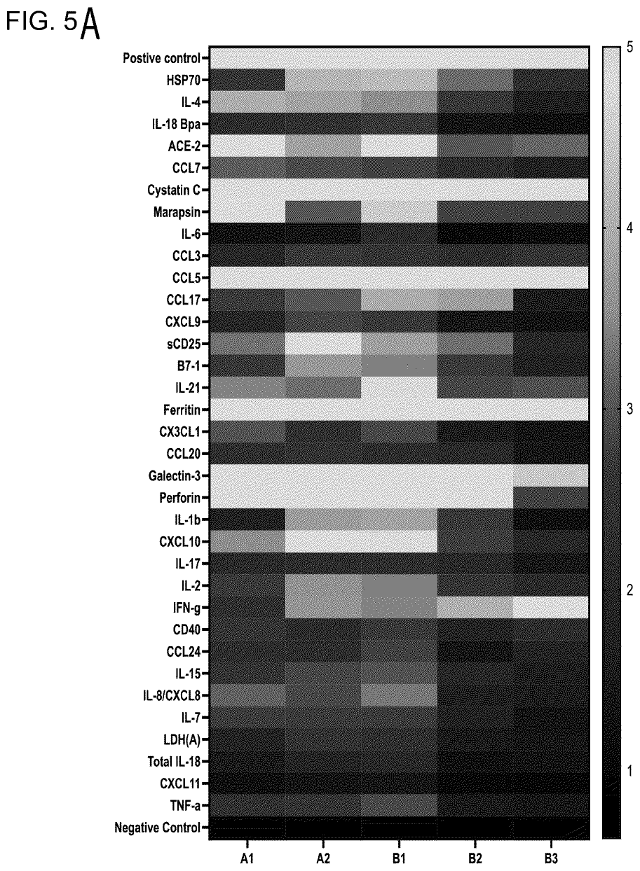

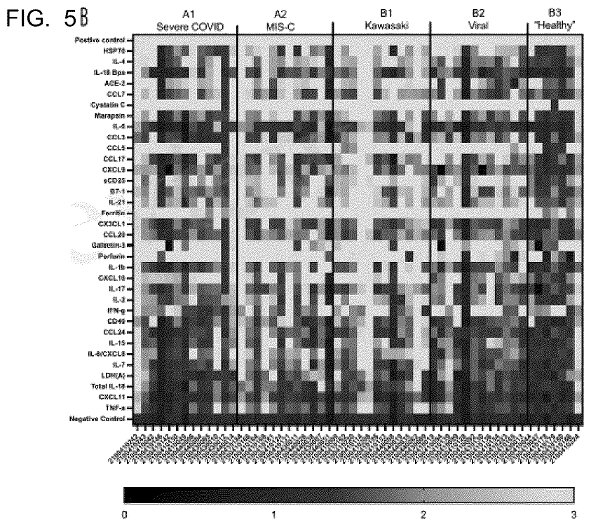

The first-generation GCFP chip was then evaluated to identify a candidate biomarker signature of disease using data from each of the 6 cohorts. Patient saliva and serum were analyzed with the biosensor chips and the fluorescent intensities were captured using GCFP reader (Ciencia, Inc., CT, USA) (FIG. 1). For each patient sample, an image was produced from the reader; Ciencia software was used to identify ROIs, and then the fluorescent intensities for each ROI were measured and the corresponding detection ratios calculated. FIG. 3 shows representative saliva data generated from a randomly selected patient from cohort B2. The image generated from patient ID 210041005 shows that five capture antibodies and the two positive controls (Biotin-BSA and alexafluor-647 labeled BSA) have a positive signal (FIG. 3A). Cystatin C, Marapsin, CRP, Galectin-3, and IL-8 ROIs were each significantly different from the negative control, indicating that these analytes are present at high levels in this patient's saliva (FIG. 3B). The fluorescent intensities were then normalized to the negative control and a heat map was generated to visualize the biomarker signature (FIG. 3C). Cystatin C, Marapsin, CRP, Galectin-3, and IL-8 all had high detection ratios. Saliva from the six cohorts (A1=15, A2=9, A3=6, B1=4, B2=13, B3=7) were similarly analyzed, and results are displayed as heatmaps (FIG. 4A/B). FIG. 4A shows the mean for each analyte from all the patient samples in each of the 6 cohorts. From this initial small data set we can begin to identify potential biomarkers for A2 (CXCL10), A3 (sDC25) and B1 (IL-1β and IL-2). Interestingly, when comparing individual patients from each cohort we can see variation in these biomarker signatures, suggesting variation in disease state presentation (FIG. 4B). From the serum cohorts, 56 samples (A1=13, A2=12, B1=12, B2=12, B3=7) were analyzed with the first-generation biosensor chip configuration and heat maps were generated for each group and for each individual patient (FIG. 5A and FIG. 5B). FIG. 5A shows the mean for each analyte from all the patient samples run in each of the 5 cohorts and indicates potential biomarker signatures for each cohort. In A2 (MIS-C group) biomarker sCD25 was found to be a potential unique biomarker for this group, while IL-21 was a unique biomarker for B1 (Kawasaki). Again, variation between individuals' biomarker signatures within the different cohorts indicates heterogeneous disease presentation (FIG. 5B).

Interrogation of GCFP Saliva Outliers Using 16S-23S rRNA Gene Amplicon Sequence Analysis

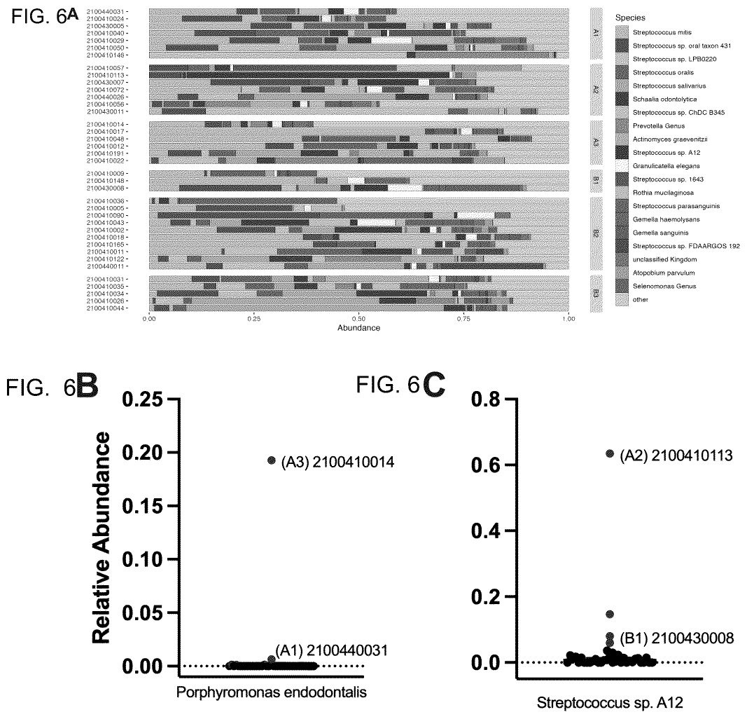

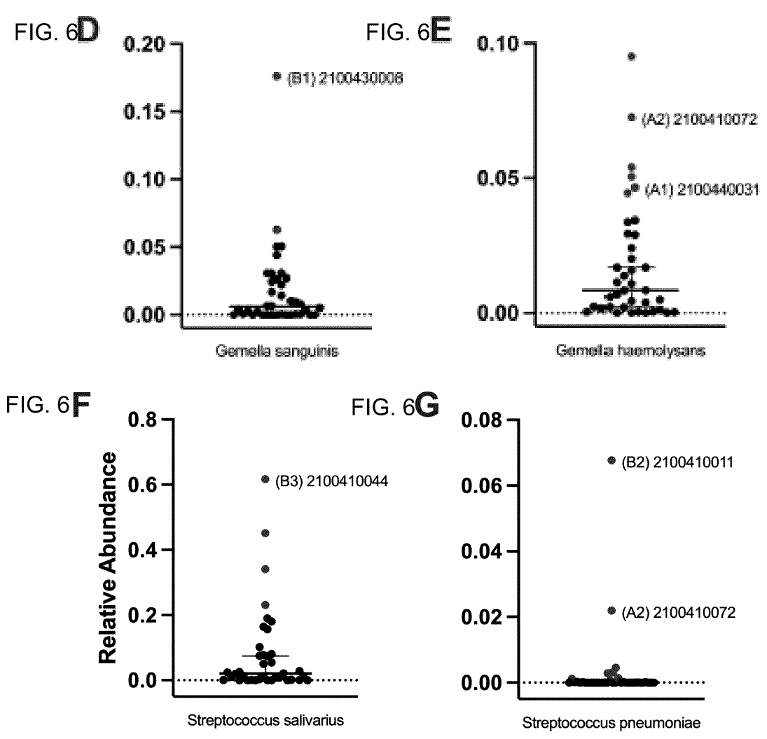

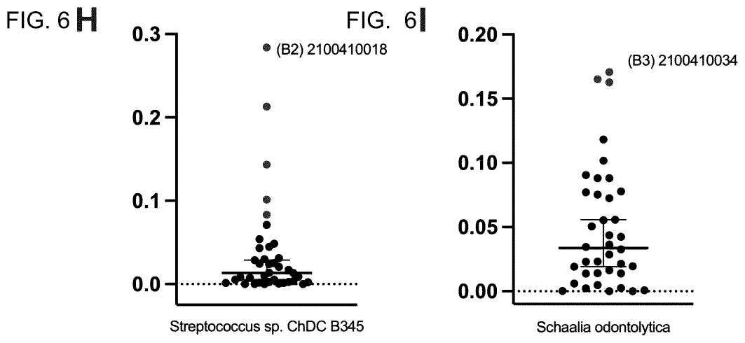

Some analyte outliers were noted within individual saliva samples (FIG. 4 B). To further analyze the saliva from these samples, 16S-23S′ rRNA PacBio sequencing was performed. For this study, sequencing data was singled out for the samples that were run over the first-generation biosensor chip and compared the relative abundances of different bacterial species. To identify bacterial species that were more abundant in the samples that were analyte outliers, a 99% confidence interval was applied, and confirmed outliers with the ROUT method. These data revealed a difference in relative abundance of several bacterial species, some of which could be linked to immunocompromised status, oral hygiene or other diseases (FIG. 6). For example, sample 2100410014 from cohort A3 had an inflammatory profile that more closely resembled a sample from the MIS-C or Kawasaki cohorts. This sample also had highly elevated levels of Porphorymonas endodontalis, a known oral pathogen, with a relative abundance of 19.26% of the overall community (median=0.0%) (FIG. 6 B). In sample 2100410113, another saliva sample that was an outlier in the biomarker analysis, it was found that the community was composed primarily of Streptococcus sp. A12 (63.45%, median=0.74%) (FIG. 6 C). Sample 2100430008 had high levels of Gemella sanguinis (17.61%, median=0.59%) and Streptococcus sp. A12 (8.00%, median=0.74%) (FIGS. 6 C and D). Sample 2100440031 had raised levels of P. endodontalis (0.64%, median=0.0%) and Gemella haemolysans (4.65%, median=0.84%) (FIGS. 6 B and 6 E). In sample 2100410044, the majority of the community was Streptococcus salivarius (61.67%, median=2.05%) (FIG. 6 F). Sample 2100410011 had elevated levels of Streptococcus pneumoniae (6.77%, median=0.0%) (FIG. 6 G). 2100410072 also had elevated levels of S. pneumoniae (2.20%, median 0.0%), as well as above average levels of G. haemolysans (7.26%, median=0.84%) (FIGS. 6 E and 6 G). In sample 2100410018, we observed elevated levels of Streptococcus sp. ChDC B345 (28.39%, median=1.34%) (FIG. 6 H). Our last GCFP analyte outlier, sample 2100410034, had elevated levels of S. salivarius (18.09%, median=2.05%) and Schaalia odontolytica (17.07%, median=3.36%) (FIGS. 6 F and 6 I).

### Refining the Composition of a Second-Generation Chip Using MIA

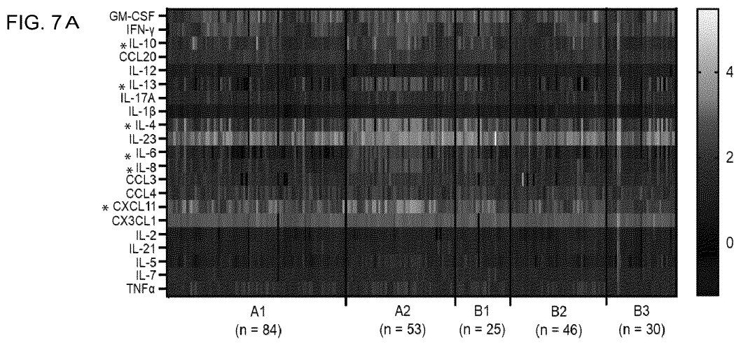

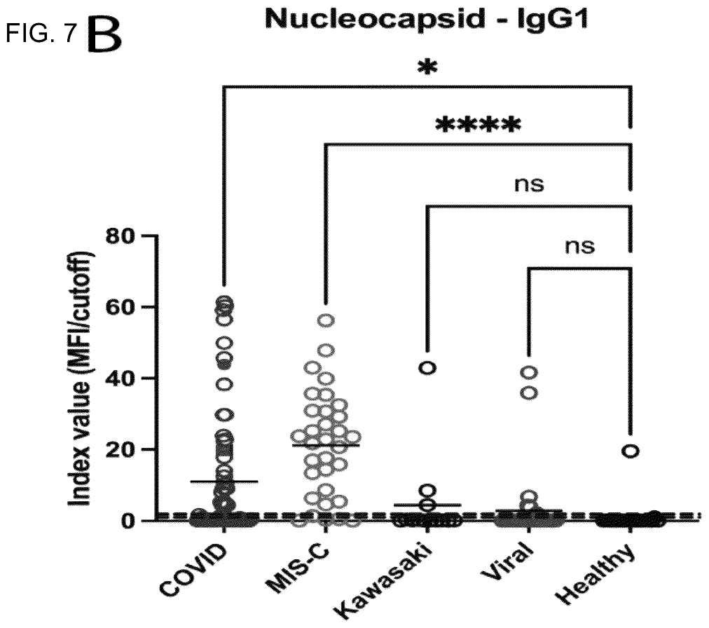

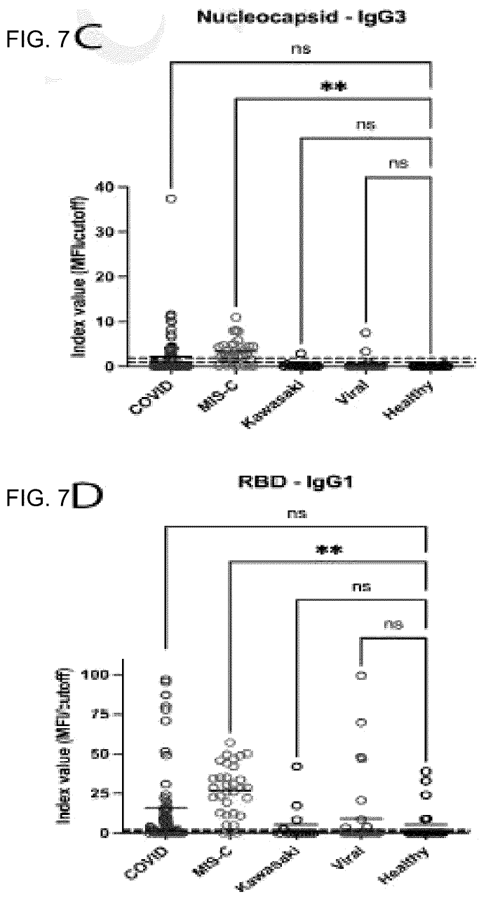

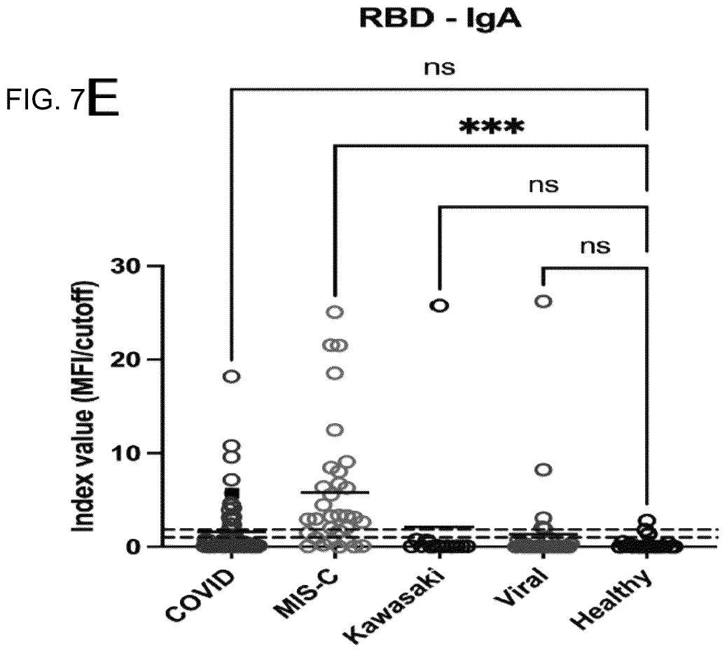

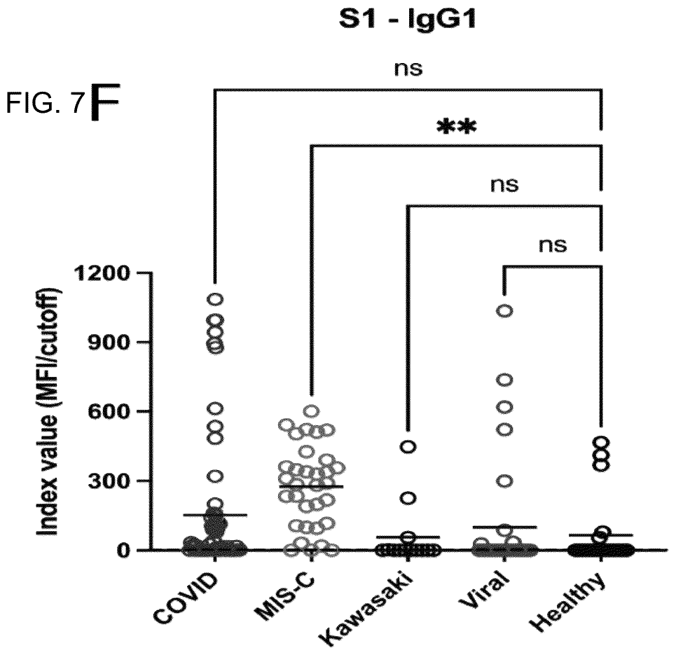

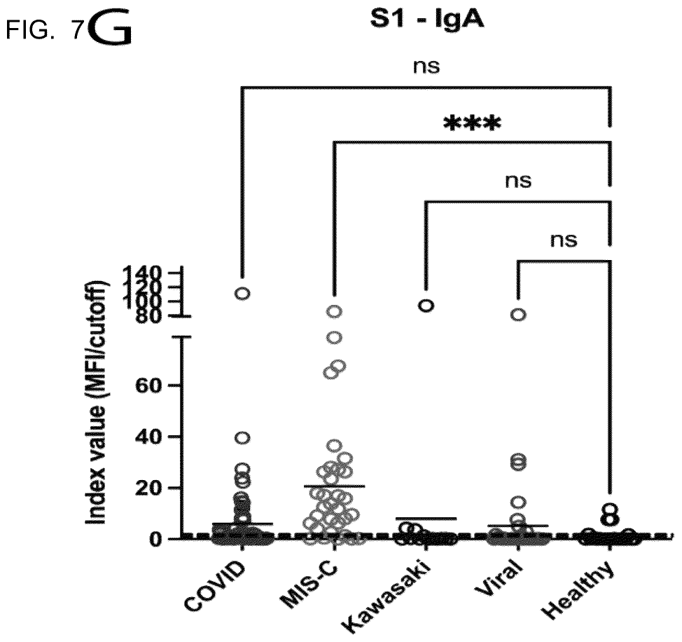

Concurrent with these microarray studies, candidates for inclusion on a second generation GCFP sensor chip were evaluated. 249 serum samples (180 from the USA and 69 from Colombia) from five different cohorts (A1, A2, B1, B2 and B3) were examined. Patient samples were analyzed for levels of 26 cytokines/chemokines using the Luminex xMAP® multiplex assay. IL-4, IL-6, IL-10, and IL-13, known to be involved in Th₂type immune response and promoting B cell differentiation to plasma cells, were significantly elevated in the A2 (MIS-C cohort) when compared to most other cohorts (FIG. 7A). Furthermore, chemokines IL-8 and CXCL11 were also found to be expressed at higher levels in the A2 cohort. By comparison, the first-generation GCFP chip detected higher IL-8 for cohorts A1, A2, B1 and IL-4 in cohorts A1 and A2 (FIG. 5A), while it did not detect IL-6 for any of the cohorts. It is important to note that MIA may use different proprietary antibodies or conditions or recognize different epitopes on these targets. The results from the Luminex studies were representative of a higher sample size than the GCFP analysis, which may also contribute to differences in the results. MIA data did confirm IL-10, IL-13 and CXCL11 as possible new targets for the second-generation chip.

The antibody response to the SARS-CoV-2 nucleocapsid and spike components (full spike, RBD, S1, S2) were measured using a MIA to separately detect anti-viral IgM, IgA, and all four IgG subclasses. FIG. 7B-7G shows that responses in the MIS-C cohort were substantially different from standard COVID-19 infections and from healthy controls. Note that some sera in the control cohort had antibodies to either the spike proteins or nucleocapsid, indicating previously undetected SARS-CoV-2 infection or an unreported vaccination. The predominant IgG subclasses produced in response to COVID-19 infection are IgG₁; both the COVID-19 and MIS-C cohorts had prominent IgG₁ response centered around the RBD/S1 components of SARS-CoV-2 spike protein. The MIS-C cohort generally made a stronger IgG₁ response as compared to the COVID-19 cohort. Notably, the MIS-C cohort made a significantly higher IgA response to SARS-COV-2 spike domains as compared to the COVID-19 cohort (FIG. 7G), indicating SARS-COV-2 spike antigen as a possible target for the second generation GCFP chip.

### Comparison of GCFP Biosensor Chip Biomarker Signature to Cytokine MIA Results

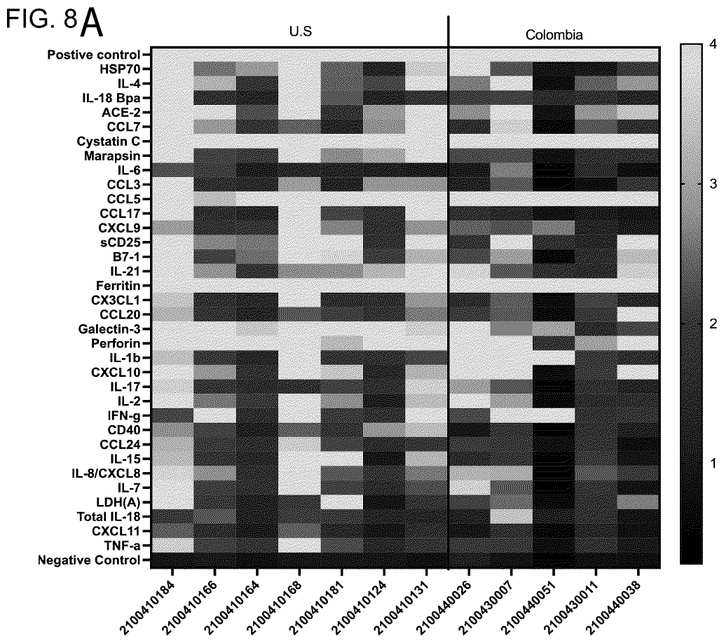

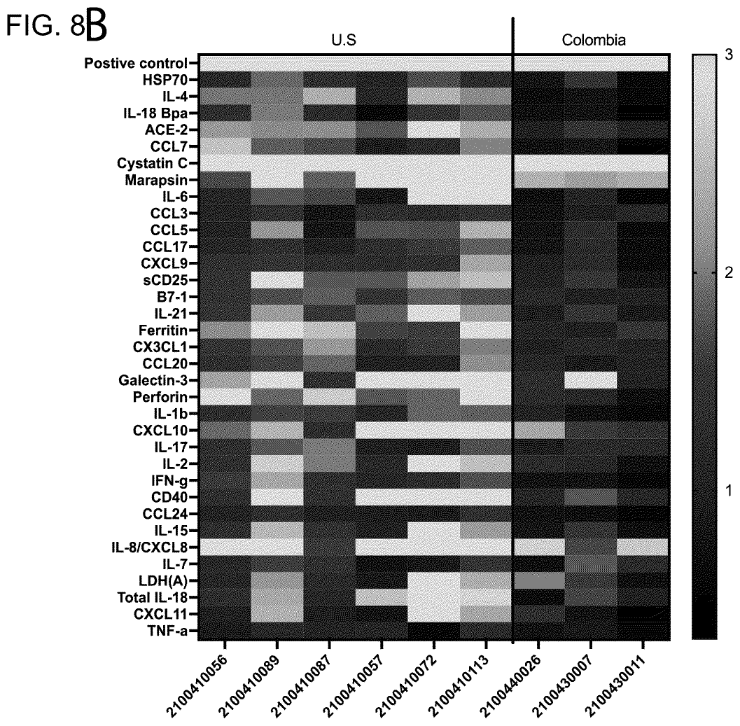

US serum samples were compared with Colombia serum samples using both GCFP biomarker signature data and cytokine microbead assay (FIG. 8A and Table 2). In cohort A2 (MIS-C), the biomarkers detected by the GCFP chip in US serum samples produced a more complex and robust signature when compared to the Colombia serum samples (FIG. 8A). US patient saliva samples were also compared with Colombia saliva samples: biomarker signatures produced by GCFP microarray had a trend similar to that observed with serum samples (FIG. 8B). Correspondingly, MIA performed on serum samples showed lower levels of cytokine in Cohort A2 Colombia samples compared to US samples (Table 2). Taken together these data indicate a difference in immune response between these two populations.

TABLE 2
MIA Cytokine levels US vs Colombia

| Cytokine | A1 | | A2 | | B1 | | B2 | |
|---|---|---|---|---|---|---|---|---|
| (pg/mL) | US | Colombia | US | Colombia | US | Colombia | US | Colombia |
| CXCL11 | 262.8 | 359.2 | 1123 | 520.2 | 379.3 | 466.5 | 181 | 195.6 |
| GM-CSF | 120.6 | 239.4 | 203.1 | 163.2 | 250.3 | 291.4 | 111.6 | 228.7 |
| CX3CL1 | 236.1 | 246.3 | 272 | 254.2 | 246.9 | 268.3 | 213.6 | 244.9 |
| IFN-γ | 105 | 113.1 | 137.3 | 150.4 | 92.00 | 87.81 | 56.72 | 106.5 |
| IL-10 | 46.3 | 174.6 | 187.4 | 157 | 71.07 | 79.41 | 26.82 | 75.5 |
| CCL20 | 33.2 | 49.1 | 59.7 | 151.4 | 43.72 | 64.38 | 27.83 | 42.6 |
| IL-12(p70) | 5.3 | 7.6 | 5.2 | 5.9 | 6.6 | 5.2 | 6.052 | 6.3 |
| IL-13 | 34.1 | 14.03 | 119.2 | 24.53 | 114.4 | 20.26 | 19.98 | 27.4 |
| IL-17A | 21.9 | 24.18 | 26.2 | 18.50 | 27.45 | 21.86 | 15.84 | 25.8 |
| IL-1β | 3.7 | 4.1 | 5 | 4.3 | 5.4 | 4.3 | 3.779 | 4.3 |
| IL-2 | 6 | 7.4 | 6.8 | 6.4 | 6.9 | 7.1 | 4.4 | 7.3 |
| IL-21 | 7.1 | 9.2 | 10.1 | 7.1 | 10.17 | 11.80 | 7.2 | 11 |
| IL-4 | 250 | 71.05 | 689.4 | 125.6 | 532.0 | 106.6 | 146.0 | 129 |
| IL-23 | 1103 | 792.4 | 1302 | 768.8 | 1530 | 43016 | 532.5 | 1031 |
| IL-5 | 8.4 | 6.068 | 19.4 | 7.8 | 20.49 | 6.8 | 24.93 | 6.6 |
| IL-6 | 27.3 | 8.9 | 77.0 | 150.4 | 61.24 | 12.35 | 23.08 | 11.28 |
| IL-7 | 15 | 14.14 | 18.5 | 15.5 | 18.59 | 30.50 | 13.68 | 14.66 |
| IL-8 | 39.6 | 18.3 | 104 | 35.9 | 76.84 | 29.86 | 24.11 | 29.98 |
| CCL3 | 32.7 | 43.86 | 48.5 | 26.4 | 37.11 | 28.67 | 73.93 | 32.95 |
| CCL4 | 45.7 | 54.39 | 64.8 | 47.61 | 58.97 | 61.19 | 44.98 | 69.87 |
| TNF-α | 16.5 | 30.7 | 33.4 | 35.2 | 27.31 | 33.32 | 19.92 | 33.33 |
| IFN-α2 | 124.7 | 227 | 214 | 153.6 | 267.7 | 183.5 | 465.2 | 234.5 |
| IL-15 | 27.8 | 17.3 | 26.6 | 11.94 | 16.58 | 6.47 | 22.66 | 9.795 |
| IL-18 | 270.7 | 362.7 | 835 | 1846 | 599.4 | 507.3 | 574.5 | 557.6 |
| IL-33 | 422.8 | 85.4 | 831.4 | 163.2 | 930.3 | 712 | 355.5 | 250.4 |
| IFN-β | 1421 | 527 | 3446 | 468 | 3158 | 156.8 | 2750 | 647.4 |

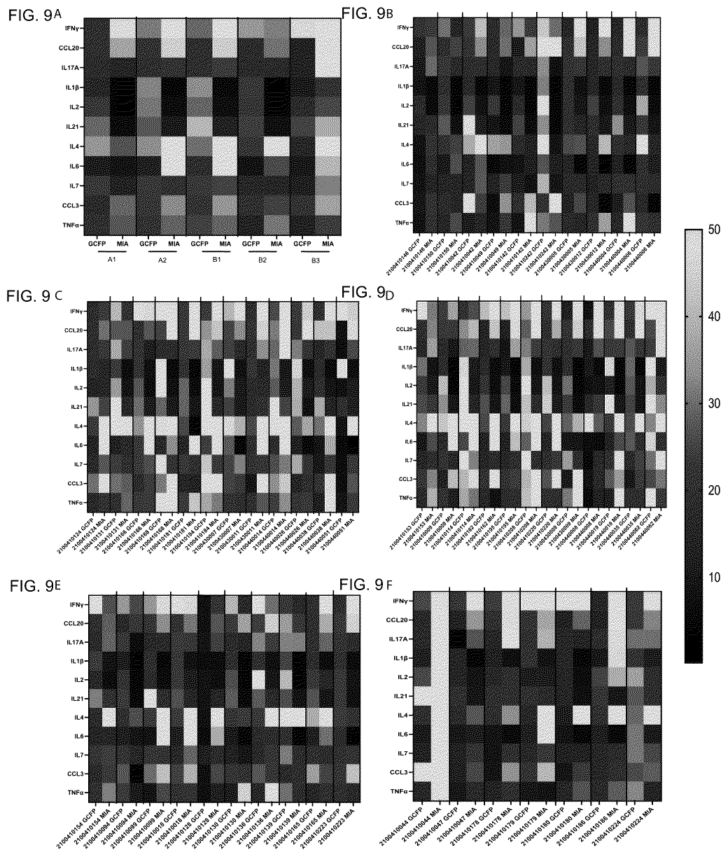

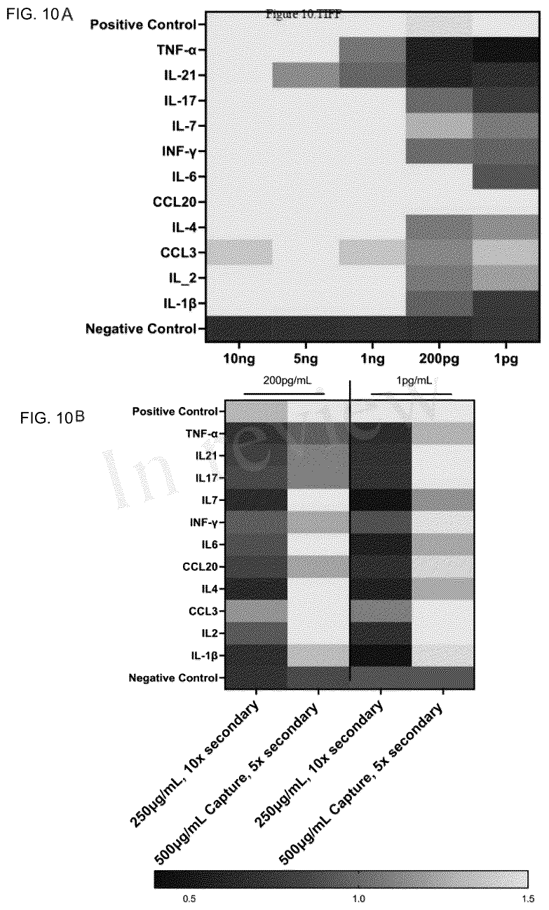

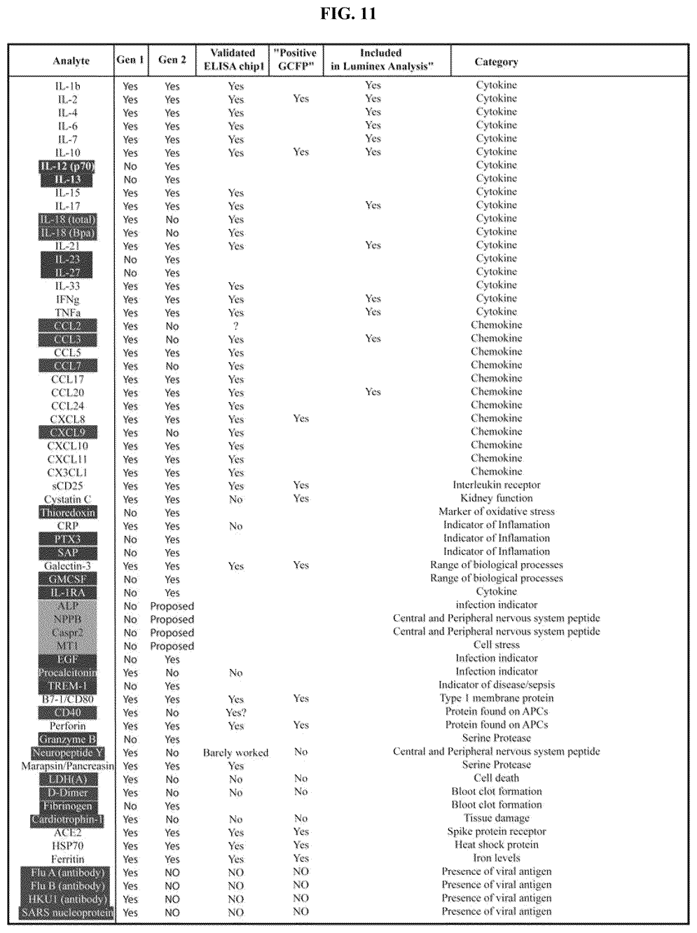

To compare MIA data directly with the biomarker signature generated on the first-generation chip, 11 analytes that had been included in both assays and detection ratios were log transformed to visualize the data by cohort and as individual patients (FIG. 9). The heat map reveals that most of the analytes were detected within both biomarker signatures generated from MIA and GCFP for the cohorts with the exception of IL-4, IL-6, CCL20 and IL-21. When individual samples were compared, similar signature patterns were found in the following samples for both MIA and GCFP; 2100410146, 2100410150, 2100410042, 2100410049, 2100410131, 2100410168, 2100410184, 2100440014, 2100410153, 2100410114, 2100410208, 2100440008, 21004400062, 2100410094, 2100410139, and 2100410165. When noting the quantified analyte levels (pg/mL) in the microbead assay dataset (Table 3) it also becomes clear that some of the analytes (IL-4, IL-6 and CCL20) were not detected on the GCFP chip while others, like IL-1β, were detected on the chip but not within the microbead assay. To address this, a dose response curve was performed on the first-generation chip for analytes that were measured both by MIA and GCFP using PBS that was doped with different amounts of recombinant proteins (10 ng, 5 ng, 1 ng, 200 pg, 1 pg/mL) (FIG. 10A). These data indicate that for many of the analytes analyzed by both MIA and GCFP, the first-generation chip has a detection limit of 1 ng/mL while MIA could detect levels as low as 1 pg/mL, which could explain the analytes that were undetected by GCFP. The GCFP chip was further optimized to improve the assay's limit of detection (FIG. 10B). The amount of secondary antibody was first increased to 10-fold the amount recommended by the manufacturers for ELISA. No improvement was observed with this change (FIG. 10B). The amount of capture antibody immobilized on the GCFP chip was then increased by twofold (500 μg/mL) and an improvement was seen in limit of detection for most analytes, indicating that manipulating capture reagents can further improve detection limits.

TABLE 3
MIA (pg/mL) data for individual samples run over GCFP chip

| Co- | | | | | | | | | | | | |
|---|---|---|---|---|---|---|---|---|---|---|---|---|
| hort | Study ID | IFNγ | CCL20 | IL17A | IL1β | IL2 | IL21 | IL4 | IL6 | IL7 | CCL3 | TNFα |
| A1 | 2100410146 | 17.99 | 22.46 | 25.39 | 2.64 | 3.09 | 11.67 | 15.17 | 12.17 | 8.63 | 15.05 | 14.70 |
| A1 | 2100410150 | 9.73 | 19.91 | 11.26 | 1.74 | 1.37 | 4.41 | 19.56 | 21.61 | 9.23 | 9.61 | 21.01 |
| A1 | 2100410042 | 41.67 | 29.65 | 12.21 | 2.64 | 5.26 | 7.64 | 113.49 | 22.78 | 23.18 | 19.02 | 9.52 |
| A1 | 2100410049 | 24.95 | 26.19 | 6.26 | 1.12 | 2.17 | 3.09 | 32.05 | 4.76 | 18.65 | 30.83 | 16.79 |
| A1 | 2100410142 | 17.99 | 34.28 | 10.61 | 1.77 | 1.42 | 3.30 | 8.15 | 7.88 | 5.08 | 40.25 | 76.752 |
| A1 | 2100410242 | 25.75 | 81.473 | 4.396 | 1.387 | 2.145 | 2.513 | 5.588 | 7.747 | 6.054 | 89.34 | 22.512 |
| A1 | 2100430005 | 160.96 | 42.857 | 16.276 | 4.485 | 6.169 | 9.594 | 21.965 | 1.851 | 11.10 | 20.86 | 9.730 |
| A1 | 2100430012 | 98.53 | 29.886 | 13.551 | 2.921 | 4.223 | 6.641 | 40.445 | 0.469 | 10.746 | N/A | 33.91 |
| A1 | 2100440004 | 73.92 | 85.147 | 11.612 | 2.028 | 3.958 | 6.704 | 10.843 | 1.017 | 14.177 | 22.327 | 66.209 |
| A1 | 2100440006 | 102.4 | 54.861 | 16.928 | 2.166 | 3.241 | 5.989 | 9.478 | 7.557 | 11.603 | N/A | 14.205 |
| A2 | 2100410124 | 18.11 | 26.395 | 16.944 | 10.07 | 9.818 | 20.012 | 1551.95 | 96.419 | 22.838 | 53.882 | 14.726 |
| A2 | 2100410131 | 14.476 | 22.469 | 19.100 | 3.857 | 5.467 | 10.558 | 56.216 | 11.339 | 13.197 | 23.880 | 24.944 |
| A2 | 2100410166 | 46.926 | 23.086 | 7.705 | 4.511 | 3.618 | 7.178 | 809.864 | 104.99 | 21.764 | 18.846 | 18.587 |
| A2 | 2100410168 | 79.440 | 50.762 | 11.867 | 3.121 | 4.621 | 6.697 | 373.516 | 29.899 | 15.215 | 53.326 | 77.089 |
| A2 | 2100410181 | 185.01 | 213.47 | 18.342 | 2.033 | 3.622 | 6.097 | N/A | 117.89 | 14.138 | 8.607 | 43.378 |
| A2 | 2100410184 | 77.082 | 43.181 | 23.771 | 6.112 | 6.263 | 10.492 | 1851.387 | 157.80 | 17.922 | 80.809 | 27.508 |
| A2 | 2100430007 | 176.81 | 30.191 | 11.265 | 4.846 | 6.726 | 6.867 | 38.369 | 2.301 | 28.318 | 17.555 | 12.542 |
| A2 | 2100430011 | 240.33 | 45.680 | 30.538 | 8.766 | 10.634 | 14.154 | 727.893 | 70.828 | 16.462 | 52.530 | 22.878 |
| A2 | 2100440014 | 233.69 | 51.139 | 52.661 | 6.494 | 7.062 | 20.039 | 605.918 | 58.722 | 27.530 | 43.913 | 29.888 |
| A2 | 2100440026 | 44.562 | 54.320 | 15.369 | 3.987 | 7.131 | 4.836 | 35.421 | 2.913 | 15.392 | 13.605 | 17.698 |
| A2 | 2100440038 | 53.690 | 42.572 | 14.926 | 3.344 | 6.066 | 8.820 | 101.331 | 36.293 | 16.344 | 47.104 | 57.186 |
| A2 | 2100440051 | 44.870 | 47.104 | 21.017 | 4.074 | 7.494 | 7.807 | 95.682 | N/A | 17.747 | 22.342 | 18.613 |
| B1 | 2100410153 | 28.274 | 29.544 | 34.508 | 7.469 | 8.766 | 18.131 | 119.172 | 22.697 | 14.948 | 29.633 | 38.459 |
| B1 | 2100410009 | 35.193 | 18.968 | 8.138 | 2.112 | 2.864 | 8.191 | 500.769 | 87.089 | 11.799 | 41.518 | 9.805 |
| B1 | 2100410114 | 22.686 | 40.462 | 26.170 | 8.919 | 8.393 | 10.674 | 528.417 | 95.820 | 29.197 | 47.817 | 27.935 |
| B1 | 2100410162 | 70.421 | 43.182 | 14.606 | 5.674 | 5.575 | 7.928 | 1414.727 | 162.38 | 13.269 | 63.126 | 6.663 |
| B1 | 2100410195 | 97.374 | 47.371 | 34.842 | 3.833 | 4.992 | 5.886 | 79.249 | 42.343 | 14.093 | 20.295 | 15.018 |
| B1 | 2100410208 | 95.327 | 73.538 | 20.004 | 6.121 | 7.852 | 13.233 | 786.041 | 116.84 | 15.472 | 61.708 | 70.394 |
| B1 | 2100410220 | 67.378 | 37.695 | 14.904 | 6.210 | 6.842 | 9.034 | 920.476 | 105.73 | 18.896 | 50.727 | 14.473 |
| B1 | 2100430009 | 82.884 | 44.793 | 19.078 | 2.806 | 3.487 | 5.243 | 39.007 | 8.088 | 13.860 | 59.329 | 85.504 |
| B1 | 2100440008 | 147.72 | 54.735 | 23.168 | 4.793 | 7.378 | 18.486 | 24.782 | 5.283 | 12.411 | 12.756 | 9.961 |
| B1 | 2100440019 | 93.395 | 63.587 | 19.383 | 4.485 | 4.485 | 8.318 | 267.711 | 22.617 | 55.529 | 33.939 | 57.482 |
| B1 | 2100440035 | 47.245 | 51.832 | 17.975 | 3.690 | 7.538 | 7.582 | 76.842 | 14.062 | 20.910 | 19.586 | 14.467 |
| B1 | 2100440062 | 109.14 | 136.29 | 48.415 | 9.281 | 16.410 | 30.346 | 143.985 | 15.336 | 83.314 | 27.953 | 18.938 |
| B2 | 2100410154 | 22.025 | 24.148 | 24.724 | 5.570 | 4.078 | 15.106 | 74.507 | 9.774 | 19.226 | 18.072 | 28.337 |
| B2 | 2100410094 | 20.910 | 17.062 | 3.788 | 1.199 | 2.392 | 2.874 | 21.835 | 6.313 | 10.235 | <0.305 | 7.194 |
| B2 | 2100410099 | 49.655 | 29.979 | 10.854 | 2.891 | 3.559 | 12.365 | 724.661 | 141.28 | 15.744 | 42.326 | 20.866 |
| B2 | 2100410018 | 47.011 | 27.137 | 16.901 | 5.279 | 7.557 | 9.855 | 235.516 | 56.305 | 25.039 | 38.238 | 15.938 |
| B2 | 2100410128 | 16.181 | 22.582 | 19.700 | 3.858 | 4.288 | 9.512 | 319.566 | 37.435 | 11.807 | 20.904 | 8.048 |
| B2 | 2100410130 | 11.098 | 34.294 | 9.368 | 1.941 | 1.608 | 2.973 | 18.721 | 2.563 | 6.438 | 15.019 | 45.419 |
| B2 | 2100410136 | 26.261 | 45.103 | 18.858 | 4.858 | 6.404 | 11.251 | 80.881 | 12.048 | 16.326 | 16.604 | 105.15 |
| B2 | 2100410139 | 11.097 | 17.000 | 27.980 | 1.808 | 1.618 | 6.139 | 54.288 | 2.250 | 12.061 | 12.735 | 12.080 |
| B2 | 2100410165 | 85.133 | 37.517 | 25.051 | 4.111 | 10.111 | 6.487 | 169.607 | 26.538 | 11.999 | 19.993 | 13.895 |
| B2 | 2100410223 | 54.958 | 45.731 | 11.988 | 3.778 | 5.668 | 4.532 | 14.752 | 0.794 | 15.679 | 42.472 | 11.174 |
| B2 | 2100440013 | N/A | 62.422 | <0.732 | <0.488 | <0.488 | <0.244 | <1.831 | 0.648 | 3.655 | 44.673 | 98.610 |
| B3 | 2100410044 | 847.51 | 177.361 | 344.704 | 63.319 | 104.810 | 260.294 | 1775.450 | 227.8 | 155.94 | 147.66 | 84.095 |
| B3 | 2100410047 | 76.169 | 32.096 | 22.405 | 2.237 | 5.951 | 6.380 | 22.931 | 7.534 | 15.576 | 20.622 | 10.982 |
| B3 | 2100410178 | 177.42 | 48.643 | 54.235 | 4.733 | 13.264 | 12.927 | 30.395 | 3.337 | 13.307 | 31.184 | 13.247 |
| B3 | 2100410179 | 125.91 | 35.238 | 38.669 | 6.024 | 7.252 | 10.763 | 815.693 | 83.032 | 17.261 | 39.916 | 5.621 |
| B3 | 2100410180 | 53.190 | 24.635 | 8.838 | 2.574 | 3.983 | 4.709 | 49.154 | 2.771 | 8.421 | 18.471 | 22.288 |
| B3 | 2100410186 | 124.61 | 56.263 | 49.416 | 46.751 | 37.120 | 6.276 | 220.268 | 3.001 | 11.761 | 12.932 | 23.911 |
| B3 | 2100410224 | 106.05 | 28.513 | 26.226 | 7.338 | 12.138 | 12.463 | 52.223 | 7.092 | 20.613 | 23.508 | 9.046 |

### Discussion

Clinicians need a rapid, accurate test for MIS-C to guide treatment decisions. Herein disclosed is that GCFP technology can be used to characterize two biomarker signatures within two different biofluids (serum and saliva), one for a group/cohort and one for an individual patient. In saliva, the GCFP biomarker analytes found in all disease state cohorts were Cystatin C, Marapsin, Galactin-3, and IL-8. When designing the chip Cystatin C was included for its potential as being predicative of renal injury and due to its observed elevated levels in COVID-19 patients. Cystatin C was detected in all patient cohorts; only one patient did not produce detectable Cystatin C. The normal range of Cystatin C is 10-12 mg/mL in saliva and, 0.63-8.0 mg/mL in serum; and is upregulated in people with poor oral hygiene. This suggested that Cystatin C may present at levels exceeding the upper limit of GCFP detection under the conditions tested. Marapsin/pancreasin is a trypsin-like serine protease that has been described in pancreatic tissue; little is known about its role in other systems although it can be expressed in stratified squamous epithelium tissues (esophagus, cervix and larynx). This analyte was included to potentially predict pancreatic health since reduced pancreatic function increases risk of developing severe COVID-19. The developed biosensor chip was able to detect marapsin in all of the infected cohorts (A1-3 & B1-2), but not in the healthy cohort (B3). Interestingly, marapsin is upregulated in the epidermis of patients with psoriasis and/or regenerating wound infections, suggesting a possible role in infection. Galectin-3 is a β-galactoside binding lectin that can drive neutrophil chemotaxis, bind TLR4, and increase production of pro-inflammatory cytokines during viral infections. Increased serum levels of galectin-3 (>30 ng/mL) have been correlated with severe COVID-19 outcomes. Disclosed biomarker signature showed galectin-3 was present in all cohorts, while lower in healthy controls, indicating a role in infection. Galectin-3 may be upregulated in COVID-19 cohorts (A1,2,3) but the amount found in saliva were near saturation for the GCFP assay as configured. Finally, IL-8 cytokines are produced by a wide range of cells including oral keratinocytes and are a potential biomarker for predicting oral disease. IL-8 was also reported early in the pandemic to be a sensitive serum biomarker in both mild and severe COVID-19 patients. Interestingly, GCFP detected salivary IL-8 in all cohorts, while in serum it was detected only in the A1, A2 and B1 cohorts.

There were some cohort-unique saliva biomarkers contributing to the biomarker signature from each of the different cohorts. GCFP detection of CXCL10 was only seen in Cohort A2 (MIS-C). CXCL10 is a chemokine that is produced by many different cell lines and tissues. This chemokine has been identified in several studies as a contributing factor to the modulation and intensity of inflammation caused by SARS-CoV2 and is at higher levels in MIS-C patients. sCD25 was solely detected on the GFCP chip in sera from cohort A3 (mild COVID). sCD25 is the soluble form of IL-2R alpha chain, has been linked to T-cell proliferation and inflammatory disease, and is a driver of disease pathogenesis. sCD25 was reported to be upregulated in the serum of pediatric patients with SARs-CoV2 infections and MIS-C. It is interesting to note that sCD25 is present in the saliva from the A3 cohort and present in the serum biomarker signature from A1, A2, and B1 cohorts. In Kawasaki patient serum, sCD25 has been found to be 3-100× higher than in healthy controls. In cohort B1, the most diverse saliva biomarker signature was observed when compared to the other cohorts, with ferritin, IL-1β and IL-2 being uniquely found at elevated levels in this cohort. Studies have indicated that high serum levels of ferritin are present in Kawasaki patients and ferritin is a predictor of non-responsiveness to intravenous immunoglobulin (IVIG) therapy. IL-1β has previously been reported to play a key role in the inflammatory profile of Kawasaki disease, and IL-2 is significantly higher in children with Kawasaki than in healthy controls. These findings indicate that data obtained by GCFP in saliva samples are consistent with recent scientific studies and show potential for this assay as a diagnostic tool. It was demonstrated that there was variation between individual saliva samples in patients within the same cohort, indicating differences in immune responsiveness.

The microbiome 16S RNA data helped to decipher potential reasons for some of the observed differences within cohorts by demonstrating imbalances in the microbial community or the presence of oral pathogens.

Serum samples demonstrated a more complex biomarker signature than saliva samples, on both individual and inter-cohort levels (FIGS. 4 and 5), which could be due to increased analyte presence in serum compared to saliva, better preservation of analyte in serum, or the choices of analytes included on the first-generation chip. The salivary proteome has 3,074 unique human proteins and only shares 1,234 proteins with blood plasma (https://www.salivaryproteome.org). GCFP biomarker analytes found in all disease state cohorts were cystatin C, ferritin, galectin 3, and perforin. As noted above, cystatin C, galectin-3 and ferritin are all found in serum at levels >250 ng/mL which could be at the limit of saturation for these assays. As expected, perforin, a glycoprotein responsible for pore formation in cell membranes of target cells, which plays an important role in cytotoxic activity, was not a marker within our healthy control group, thus showing selectivity of disease states on the developed chip. A2 and B1 serum cohorts shared 3 analytes not found in other cohorts: HSP70, IL-1β, and IL-2. HSP70, a stress protein known to induce inflammation and has been linked to pathogenesis of Kawasaki disease was not present in A2 saliva but was present in B1 saliva samples. ACE2 (angiotensin converting enzyme-2) is a monocarboxypeptidase found within cell membranes or as a soluble protein throughout the body. It is a member of the renin-angiotensin system (RAS) and has been implicated in diabatic cardiovascular complications and chronic heart failure; elevated expression has been linked to severe COVID-19, and it is a receptor for SARS-CoV2 entry into the cell. ACE2 was detected in the serum biomarker signatures for A1, A2, and B1. While ACE2 levels in cohorts A1 and A2 are consistent with recent scientific literature, it is not a known biomarker in Kawasaki disease, despite the fact that cardiac disease (coronary artery aneurysms, myocarditis, pericarditis, congestive heart failure, pericardial effusion, and arrhythmias) is a complication in these patients. The detection of ACE2 within cohort B1 could suggest the presence of cardiac disease within individuals of this group.

Previously, the versatility of the GCFP microarray has been demonstrated in its ability to easily be manipulated with the immobilization or subtraction of different capture antibodies in the array and the potential to detect over 1000 analytes in a single assay (Marusov et al, 2012, Environmental Science & Technology, 2012, 46(1) 348-359, DOI: 10.1021/es201239f). In this study, using a small sample volume (70-80 μL) of either saliva or serum, the beginning of a biomarker signature was built for the different cohorts studied, showing that GCFP microarrays can be a potential novel approach for MIS-C disease diagnostics. The reagents used on the biosensor chip were manipulated to increase the assay's sensitivity. MIA data in this study revealed four potential new GCFP targets, IL-10, IL-13, CXCL11 and SARS-COV-2 spike antigen. Disease state variation was detected between patients within a disease group, which could potentially help with individualized diagnostics and faster treatment options per patient. These data together demonstrate GCFP microarrays as a novel, professional point of care diagnostic tool.

Embodiments disclosed here are not limiting of the subject matter and is merely exemplary. Various other components may be included and called upon for providing for aspects of the teachings herein. For example, additional materials, combinations of materials and/or omission of materials may be used to provide for added embodiments that are within the scope of the teachings herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

### INCORPORATION BY REFERENCE

All references including U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### OTHER EMBODIMENTS

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

For reasons of completeness, various aspects of the disclosure are set out in the following numbered clauses:

Clause 1. A biomarker panel for assessing a subject suspected of having or having multisystem inflammatory syndrome in children (MIS-C) or Kawasaki disease, the panel comprising a plurality of first biomarker detection agents, wherein each first biomarker detection agent specifically binds to a corresponding target biomarker, wherein the target biomarkers comprise: Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), and immunoregulatory alpha globulin (IRA).

Clause 2. The biomarker panel of clause 1, wherein the target biomarkers further comprise one or more of: B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof.

Clause 3. The biomarker panel of clause 1 or clause 2, wherein the target biomarkers further comprise one or more of: Marapsin, fractalkine (CX3CL1), C-C chemokine ligand 20 (CCL20), interleukin-21, C-C chemokine ligand 17 (CCL17), interleukin-1β (IL-1β), or any combination thereof.

Clause 4. A biomarker panel for assessing a subject suspected of having or having multisystem inflammatory syndrome in children (MIS-C) or Severe Acute Respiratory Syndrome Coronavirus 2 (SARS-CoV-2), the panel comprising a plurality of first biomarker detection agents, wherein each first biomarker detection agent specifically binds to a corresponding target biomarker, wherein the target biomarkers comprise: Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), and interleukin-23 (IL-23).

Clause 5. The biomarker panel of clause 4, wherein the target biomarkers further comprise one or more of: Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7), or any combination thereof.

Clause 6. The biomarker panel of clause 4 or clause 5, wherein the target biomarkers further comprise one or more of: Pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2), or any combination thereof.

Clause 7. A biomarker panel for assessing a subject suspected of having or having multisystem inflammatory syndrome in children (MIS-C) or respiratory syncytial virus (RSV), the panel comprising a plurality of first biomarker detection agents, wherein each first biomarker detection agent specifically binds to a corresponding target biomarker, wherein the target biomarkers comprise: Granulocyte-Macrophage Colony-Stimulating Factor (GM-CSF), Pentraxin-3 (PTX-3), Marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23).

Clause 8. The biomarker panel of clause 7, wherein the target biomarkers further comprise one or more of: Perforin (Perf), C-X-C motif chemokine ligand 11 (CXCL11), Epidermal Growth Factor (EGF), interleukin-10 (IL-10), interleukin-8 (IL-8), interleukin-21 (IL-21), interleukin-2 (IL-2), immunoregulatory alpha globulin (IRA), or any combination thereof.

Clause 9. The biomarker panel of clause 7 or clause 8, wherein the target biomarkers further comprise one or more of: Granzyme B (GRAND B), fractalkine (CX3CL1), C-C motif chemokine ligand 24 (CCL24), interleukin-7 (IL-7), C-C chemokine ligand 20 (CCL20), interleukin-6 (IL-6), C-C chemokine ligand 17 (CCL17), interleukin-4 (IL-4), interleukin-17 (IL-17), interleukin-15 (IL-15), tumor necrosis factor alpha (TNFα), interleukin-1β (IL-1β), interferon gamma (INFγ), interleukin-12 (IL-12), or any combination thereof.

Clause 10. The biomarker panel of any of clauses 1-9, wherein each first biomarker detection agent is an antibody or antibody binding fragment thereof.

Clause 11. The biomarker panel of clause 10, wherein each antibody or antibody binding fragment thereof is immobilized on a solid support.

Clause 12. The biomarker panel of clause 11, wherein the solid support is a particle, microparticle, bead, plate, well, or chip.

Clause 13. The biomarker panel of any of clauses 1-12, wherein each first biomarker detection agent is labeled with a detectable label.

Clause 14. The biomarker panel of any of clauses 1-13, wherein the panel is configured as a multiplex assay or as an ELISA assay.

Clause 15. A kit for assessing a subject suspected of having or having multisystem inflammatory syndrome in children (MIS-C) or Kawasaki disease, comprising:
a. a plurality of first biomarker detection agents, wherein each first biomarker detection agent specifically binds to a corresponding target biomarker, wherein the target biomarkers comprise: Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), and immunoregulatory alpha globulin (IRA); and
b. instructions for use of the kit.

Clause 16. The kit of clause 15, wherein the target biomarkers comprise one or more of: B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof.

Clause 17. The kit of clause 15 or clause 16, wherein the target biomarkers further comprise one or more of: Marapsin, fractalkine (CX3CL1), C-C chemokine ligand 20 (CCL20), interleukin-21, C-C chemokine ligand 17 (CCL17), interleukin-1β (IL-1β), or any combination thereof.

Clause 18. A kit for assessing a subject suspected of having or having multisystem inflammatory syndrome in children (MIS-C) or Severe Acute Respiratory Syndrome Coronavirus 2 (SARS-CoV-2), comprising:
a. a plurality of first biomarker detection agents, wherein each first biomarker detection agent specifically binds to a corresponding target biomarker, wherein the target biomarkers comprise: Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), and interleukin-23 (IL-23); and
b. instructions for use of the kit.

Clause 19. The kit of clause 18, wherein the target biomarkers comprise one or more of: Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7), or any combination thereof.

Clause 20. The kit of clause 18 or clause 19, wherein the target biomarkers comprise one or more of: Pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2), or any combination thereof.

Clause 21. A kit for assessing a subject suspected of having or having multisystem inflammatory syndrome in children (MIS-C) or respiratory syncytial virus (RSV), comprising:
a. a plurality of first biomarker detection agents, wherein each first biomarker detection agent specifically binds to a corresponding target biomarker, wherein the target biomarkers comprise: Granulocyte-Macrophage Colony-Stimulating Factor (GM-CSF), Pentraxin-3 (PTX-3), Marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23); and
b. instructions for use of the kit.

Clause 22. The kit of clause 21, wherein the target biomarkers comprise one or more of: Perforin (Perf), C-X-C motif chemokine ligand 11 (CXCL11), Epidermal Growth Factor (EGF), interleukin-10 (IL-10), interleukin-8 (IL-8), interleukin-21 (IL-21), interleukin-2 (IL-2), immunoregulatory alpha globulin (IRA), or any combination thereof.

Clause 23. The kit of clause 21 or clause 22, wherein the target biomarkers comprise one or more of: Granzyme B (GRAND B), fractalkine (CX3CL1), C-C motif chemokine ligand 24 (CCL24), interleukin-7 (IL-7), C-C chemokine ligand 20 (CCL20), interleukin-6 (IL-6), C-C chemokine ligand 17 (CCL17), interleukin-4 (IL-4), interleukin-17 (IL-17), interleukin-15 (IL-15), tumor necrosis factor alpha (TNFα), interleukin-1β (IL-1β), interferon gamma (INFγ), interleukin-12 (IL-12), or any combination thereof.

Clause 24. The kit of any of clauses 15-23, wherein the kit further comprises a plurality of second biomarker detection agents which specifically bind to the corresponding target biomarker.

Clause 25. The kit of clause 24, wherein each first biomarker detection agent, each second biomarker detection agent, or each first biomarker detection agent and each second biomarker detection agent is an antibody or antibody binding fragment thereof.

Clause 26. The kit of any of clauses 15-25, wherein each of the first biomarker detection reagents is immobilized on a solid support.

Clause 27. The kit of clause 26, wherein the solid support is a particle, microparticle, bead, plate, well, or chip.

Clause 28. The kit of any of clauses 24-27, wherein each first biomarker detection reagent is labeled with a detectable label, or the second biomarker detection reagent is labeled with a detectable label.

Clause 29. A method for assessing a subject suspected of having or having multisystem inflammatory syndrome in children (MIS-C) or Kawasaki disease, the method comprising:
a. determining a measurement of a panel of first target biomarkers in a sample obtained from a subject suspected of having multisystem inflammatory syndrome in children (MIS-C) or Kawasaki disease, wherein: (i) the measurement comprises determining an amount of each of a plurality of first target biomarkers in the panel; and (ii) the target biomarkers comprise: Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), and immunoregulatory alpha globulin (IRA);
b. calculating a relative intensity value for each first target biomarker in the panel based on the measurements in step a;
c. comparing the relative intensity values calculated in step b) to reference values for the target biomarkers; and
d. assessing the subject for MIS-C or Kawasaki disease based on the comparison made in step c.

Clause 30. The method of clause 29, wherein the first target biomarkers further comprise one or more of: B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof.

Clause 31. The method of clause 29 or clause 30, wherein the panel further comprises a plurality of second target biomarkers, wherein the second target biomarkers comprise one or more of: Marapsin, fractalkine (CX3CL1), C-C chemokine ligand 20 (CCL20), interleukin-21, C-C chemokine ligand 17 (CCL17), interleukin-1β (IL-1β), or any combination thereof.

Clause 32. The method of clause 31, wherein the method further comprises:
a. determining a measurement of each of the first target biomarkers and second target biomarkers in a sample obtained from a subject suspected of having multisystem inflammatory syndrome in children (MIS-C) or Kawasaki disease;
b. calculating a relative intensity value for each first target biomarker and each second target biomarker in the panel based on the measurements in step a;
c. comparing the relative intensity values calculated in step b) to reference values for the target first and second biomarkers; and
d. assessing the subject for MIS-C or Kawasaki disease based on the comparison made in step c.

Clause 33. A method for assessing a subject suspected of having or having multisystem inflammatory syndrome in children (MIS-C) or Severe Acute Respiratory Syndrome Coronavirus 2 (SARS-CoV-2), the method comprising:
a. determining a measurement of a panel of first target biomarkers in a sample obtained from a subject suspected of having multisystem inflammatory syndrome in children (MIS-C) or Severe Acute Respiratory Syndrome Coronavirus 2 (SARS-CoV-2), wherein: (i) the measurement comprises determining an amount of each of a plurality of first target biomarkers in the panel; and (ii) the target biomarkers comprise: Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), and interleukin-23 (IL-23);
b. calculating a relative intensity value for each first target biomarker in the panel based on the measurements in step a;
c. comparing the relative intensity values calculated in step b) to reference values for the target biomarkers; and
d. assessing the subject for MIS-C or Severe Acute Respiratory Syndrome Coronavirus 2 (SARS-CoV-2) based on the comparison made in step c.

Clause 34. The method of clause 33, wherein the first target biomarkers further comprise one or more of: Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7), or any combination thereof.

Clause 35. The method of clause 29, wherein the first target biomarkers further comprise one or more of: B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof.

Clause 36. The method of clause 34 or clause 35, wherein the panel further comprises a plurality of second target biomarkers, wherein the second target biomarkers comprise one or more of: Pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2), or any combination thereof.

Clause 37. The method of clause 36, wherein the method further comprises:
a. determining a measurement of each of the first target biomarkers and second target biomarkers in a sample obtained from a subject suspected of having multisystem inflammatory syndrome in children (MIS-C) or SARS-CoV-2;
b. calculating a relative intensity value for each first target biomarker and each second target biomarker in the panel based on the measurements in step a;
c. comparing the relative intensity values calculated in step b) to reference values for the target first and second biomarkers; and
d. assessing the subject for MIS-C or SARS-CoV-2 based on the comparison made in step c.

Clause 38. A method for assessing a subject suspected of having or having multisystem inflammatory syndrome in children (MIS-C) or respiratory syncytial virus, the method comprising:
a. determining a measurement of a panel of first target biomarkers in a sample obtained from a subject suspected of having multisystem inflammatory syndrome in children (MIS-C) or respiratory syncytial virus, wherein: (i) the measurement comprises determining an amount of each of a plurality of first target biomarkers in the panel; and (ii) the target biomarkers comprise: Granulocyte-Macrophage Colony-Stimulating Factor (GM-CSF), Pentraxin-3 (PTX-3), Marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23);
b. calculating a relative intensity value for each first target biomarker in the panel based on the measurements in step a;
c. comparing the relative intensity values calculated in step b) to reference values for the target biomarkers; and
d. assessing the subject for MIS-C or respiratory syncytial virus based on the comparison made in step c.

Clause 39. The method of clause 38, wherein the first target biomarkers further comprise one or more of: Perforin (Perf), C-X-C motif chemokine ligand 11 (CXCL11), Epidermal Growth Factor (EGF), interleukin-10 (IL-10), interleukin-8 (IL-8), interleukin-21 (IL-21), interleukin-2 (IL-2), immunoregulatory alpha globulin (IRA), or any combination thereof.

Clause 40. The method of clause 38 or clause 39, wherein the panel further comprises a plurality of second target biomarkers, wherein the second target biomarkers comprise one or more of: Granzyme B (GRAND B), fractalkine (CX3CL1), C-C motif chemokine ligand 24 (CCL24), interleukin-7 (IL-7), C-C chemokine ligand 20 (CCL20), interleukin-6 (IL-6), C-C chemokine ligand 17 (CCL17), interleukin-4 (IL-4), interleukin-17 (IL-17), interleukin-15 (IL-15), tumor necrosis factor alpha (TNFα), interleukin-1β (IL-1β), interferon gamma (INFγ), interleukin-12 (IL-12), or any combination thereof.

Clause 41. The method of clause 40, wherein the method further comprises:
a. determining a measurement of each of the first target biomarkers and second target biomarkers in a sample obtained from a subject suspected of having multisystem inflammatory syndrome in children (MIS-C) or respiratory syncytial virus;
b. calculating a relative intensity value for each first target biomarker and each second target biomarker in the panel based on the measurements in step a;
c. comparing the relative intensity values calculated in step b) to reference values for the target first and second biomarkers; and
d. assessing the subject for MIS-C or respiratory syncytial virus based on the comparison made in step c.

Clause 42. A device for assessing a subject suspected of having or having multisystem inflammatory syndrome in children (MIS-C) or Kawasaki disease, the device comprising:
a. a measuring unit for determining a relative intensity of each of a plurality of first target biomarkers in a sample obtained from a subject suspected of having MIS-C or Kawasaki disease, wherein the measuring unit comprises a detection system for detecting and measuring an amount of each of the plurality of first target biomarkers and further wherein the first target biomarkers comprise: Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), and immunoregulatory alpha globulin (IRA); and
b. an evaluation unit operably linked to the measuring unit comprising a database with stored reference values for the first target biomarkers and a data processor comprising instructions for carrying out a comparison of the relative intensity of each first target biomarker to reference values for the target biomarkers and assessing the subject based on the comparison, said evaluation unit being capable of automatically receiving the relative intensity values for each of the biomarkers from the measuring unit.

Clause 43. The device of clause 42, wherein the first target biomarkers further comprise one or more of B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof.

Clause 44. The device of clause 42 or clause 43, wherein:
a. the measuring unit determines a relative intensity of each of a plurality of first target biomarkers and second target biomarkers in the sample, wherein the measuring unit comprises a detection system for detecting and measuring an amount of each of the plurality of first target biomarkers and second target biomarkers and further wherein the second target biomarkers comprise one or more of marapsin, fractalkine (CX3CL1), C-C chemokine ligand 20 (CCL20), interleukin-21, C-C chemokine ligand 17 (CCL17), interleukin-1β (IL-1β), or any combination thereof; and
b. the evaluation unit comprises a database with stored reference values for the first target biomarkers and second target biomarkers and a data processor comprising instructions for carrying out a comparison of the relative intensity of each first target biomarker and each second target biomarker to reference values for the first and second target biomarkers and assessing the subject based on the comparison.

Clause 45. A device for assessing a subject suspected of having or having multisystem inflammatory syndrome in children (MIS-C) or Severe Acute Respiratory Syndrome Coronavirus 2 (SARS-CoV-2), the device comprising:
a. a measuring unit for determining a relative intensity of each of a plurality of first target biomarkers in a sample obtained from a subject suspected of having MIS-C or Severe Acute Respiratory Syndrome Coronavirus 2 (SARS-CoV-2), wherein the measuring unit comprises a detection system for detecting and measuring an amount of each of the plurality of first target biomarkers and further wherein the first target biomarkers comprise: Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), and interleukin-23 (IL-23); and
b. an evaluation unit operably linked to the measuring unit comprising a database with stored reference values for the first target biomarkers and a data processor comprising instructions for carrying out a comparison of the relative intensity of each first target biomarker to reference values for the target biomarkers and assessing the subject based on the comparison, said evaluation unit being capable of automatically receiving the relative intensity values for each of the biomarkers from the measuring unit.

Clause 46. The device of clause 45, wherein the first target biomarkers further comprise one or more of interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7), or any combination thereof.

Clause 47. The device of clause 45 or clause 46, wherein:
a. the measuring unit determines a relative intensity of each of a plurality of first target biomarkers and second target biomarkers in the sample, wherein the measuring unit comprises a detection system for detecting and measuring an amount of each of the plurality of first target biomarkers and second target biomarkers and further wherein the second target biomarkers comprise one or more of pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2); and
b. the evaluation unit comprises a database with stored reference values for the first target biomarkers and second target biomarkers and a data processor comprising instructions for carrying out a comparison of the relative intensity of each first target biomarker and each second target biomarker to reference values for the first and second target biomarkers and assessing the subject based on the comparison.

Clause 48. A device for assessing a subject suspected of having or having multisystem inflammatory syndrome in children (MIS-C) or respiratory syncytial virus, the device comprising:
a. a measuring unit for determining a relative intensity of each of a plurality of first target biomarkers in a sample obtained from a subject suspected of having MIS-C or respiratory syncytial virus, wherein the measuring unit comprises a detection system for detecting and measuring an amount of each of the plurality of first target biomarkers and further wherein the first target biomarkers comprise: Granulocyte-Macrophage Colony-Stimulating Factor (GM-CSF), Pentraxin-3 (PTX-3), Marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23); and
b. an evaluation unit operably linked to the measuring unit comprising a database with stored reference values for the first target biomarkers and a data processor comprising instructions for carrying out a comparison of the relative intensity of each first target biomarker to reference values for the target biomarkers and assessing the subject based on the comparison, said evaluation unit being capable of automatically receiving the relative intensity values for each of the biomarkers from the measuring unit.

Clause 49. The device of clause 48, wherein the first target biomarkers further comprise one or more of perforin (Perf), C-X-C motif chemokine ligand 11 (CXCL11), Epidermal Growth Factor (EGF), interleukin-10 (IL-10), interleukin-8 (IL-8), interleukin-21 (IL-21), interleukin-2 (IL-2), immunoregulatory alpha globulin (IRA), or any combination thereof.

Clause 50. The device of clause 48 or clause 49, wherein:
a. the measuring unit determines a relative intensity of each of a plurality of first target biomarkers and second target biomarkers in the sample, wherein the measuring unit comprises a detection system for detecting and measuring an amount of each of the plurality of first target biomarkers and second target biomarkers and further wherein the second target biomarkers comprise one or more of Granzyme B (GRAND B), fractalkine (CX3CL1), C-C motif chemokine ligand 24 (CCL24), interleukin-7 (IL-7), C-C chemokine ligand 20 (CCL20), interleukin-6 (IL-6), C-C chemokine ligand 17 (CCL17), interleukin-4 (IL-4), interleukin-17 (IL-17), interleukin-15 (IL-15), tumor necrosis factor alpha (TNFα), interleukin-1β (IL-1β), interferon gamma (INFγ), interleukin-12 (IL-12), or any combination thereof; and
b. the evaluation unit comprises a database with stored reference values for the first target biomarkers and second target biomarkers and a data processor comprising instructions for carrying out a comparison of the relative intensity of each first target biomarker and each second target biomarker to reference values for the first and second target biomarkers and assessing the subject based on the comparison.

Clause 51. The device of any of clauses 42-50, wherein the detection system comprises at least one detection agent capable of specifically detecting each of the first target biomarkers, the second target biomarkers, or both the first target biomarkers and the second target biomarkers.

Clause 52. The device of clause 51, wherein the detection agent is an antibody or antibody binding fragment thereof.

What is claimed is:

1. A biomarker panel for assessing a subject:
(a) suspected of having or having multisystem inflammatory syndrome in children (MIS-C) or Kawasaki disease, the panel comprising a plurality of first biomarker detection agents, wherein each first biomarker detection agent specifically binds to a corresponding target biomarker, wherein the target biomarkers comprise: Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), and immunoregulatory alpha globulin (IRA);
(b) suspected of having or having multisystem inflammatory syndrome in children (MIS-C) or Severe Acute Respiratory Syndrome Coronavirus 2 (SARS-CoV-2), the panel comprising a plurality of first biomarker detection agents, wherein each first biomarker detection agent specifically binds to a corresponding target biomarker, wherein the target biomarkers comprise: Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), and interleukin-23 (IL-23); or
(c) suspected of having or having multisystem inflammatory syndrome in children (MIS-C) or respiratory syncytial virus (RSV), the panel comprising a plurality of first biomarker detection agents, wherein each first biomarker detection agent specifically binds to a corresponding target biomarker, wherein the target biomarkers comprise: Granulocyte-Macrophage Colony-Stimulating Factor (GM-CSF), Pentraxin-3 (PTX-3), Marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23).

2. The biomarker panel of claim 1, wherein the target biomarkers further comprise: (a) one or more of: B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof; (b) one or more of: Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7), or any combination thereof; or (c) one or more of: Perforin (Perf), C-X-C motif chemokine ligand 11 (CXCL11), Epidermal Growth Factor (EGF), interleukin-10 (IL-10), interleukin-8 (IL-8), interleukin-21 (IL-21), interleukin-2 (IL-2), immunoregulatory alpha globulin (IRA), or any combination thereof.

3. The biomarker panel of claim 1, wherein the target biomarkers further comprise (a) one or more of: Marapsin, fractalkine (CX3CL1), C-C chemokine ligand 20 (CCL20), interleukin-21, C-C chemokine ligand 17 (CCL17), interleukin-1β (IL-1β), or any combination thereof; (b) one or more of: Pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2), or any combination thereof; or (c) one or more of: Granzyme B (GRAND B), fractalkine (CX3CL1), C-C motif chemokine ligand 24 (CCL24), interleukin-7 (IL-7), C-C chemokine ligand 20 (CCL20), interleukin-6 (IL-6), C-C chemokine ligand 17 (CCL17), interleukin-4 (IL-4), interleukin-17 (IL-17), interleukin-15 (IL-15), tumor necrosis factor alpha (TNFα), interleukin-1β (IL-1β), interferon gamma (INFγ), interleukin-12 (IL-12), or any combination thereof.

4. The biomarker panel of claim 1, wherein each first biomarker detection agent is an antibody or antibody binding fragment thereof.

5. The biomarker panel of claim 4, wherein each antibody or antibody binding fragment thereof is immobilized on a solid support.

6. The biomarker panel of claim 5, wherein the solid support is a particle, microparticle, bead, plate, well, or chip.

7. The biomarker panel of claim 1, wherein each first biomarker detection agent is labeled with a detectable label.

8. The biomarker panel of claim 1, wherein the panel is configured as a multiplex assay or as an ELISA assay.

9. A method for assessing a subject suspected of having or having (i) multisystem inflammatory syndrome in children (MIS-C) or Kawasaki disease, (ii) MIS-C or Severe Acute Respiratory Syndrome Coronavirus 2 (SARS-CoV-2), or (iii) MIS-C or respiratory syncytial virus (RSV), the method comprising:
a. determining a measurement of a panel of first target biomarkers in a sample obtained from a subject (i) suspected of having multisystem inflammatory syndrome in children (MIS-C) or Kawasaki disease, wherein: (a) the measurement comprises determining an amount of each of a plurality of first target biomarkers in the panel; and (b) the target biomarkers comprise: Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), and immunoregulatory alpha globulin (IRA); (ii) suspected of having MIS-C or SARS-CoV-2, wherein: (a) the measurement comprises determining an amount of each of a plurality of first target biomarkers in the panel; and (b) the target biomarkers comprise: Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), and interleukin-23 (IL-23); or (iii) suspected of having MIS-C or RSV, wherein: (a) the measurement comprises determining an amount of each of a plurality of first target biomarkers in the panel; and (b) the target biomarkers comprise: Granulocyte-Macrophage Colony-Stimulating Factor (GM-CSF), Pentraxin-3 (PTX-3), Marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23);
b. calculating a relative intensity value for each first target biomarker in the panel based on the measurements in step a;
c. comparing the relative intensity values calculated in step b) to reference values for the target biomarkers; and
d. assessing the subject for (i) MIS-C or Kawasaki; (ii) MIS-C or SARS-CoV-2; or (iii) MIS-C or RSV disease based on the comparison made in step c.

10. The method of claim 9, wherein the first target biomarkers further comprise (a) one or more of: B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof; (b) one or more of: Interleukin-4 (IL-4), chemokine ligand 5 (CCL5), interleukin-12 (L-12), C-C chemokine ligand 20 (CCL20), interleukin-21 (IL-21), B-lymphocyte activation antigen B7 (B7-1), interleukin-8 (IL-8), interleukin-7 (IL-7), or any combination thereof; or (c) one or more of: Perforin (Perf), C-X-C motif chemokine ligand 11 (CXCL11), Epidermal Growth Factor (EGF), interleukin-10 (IL-10), interleukin-8 (IL-8), interleukin-21 (IL-21), interleukin-2 (IL-2), immunoregulatory alpha globulin (IRA), or any combination thereof.

11. The method of claim 9, wherein the panel further comprises a plurality of second target biomarkers, wherein the second target biomarkers comprise (a) one or more of: Marapsin, fractalkine (CX3CL1), C-C chemokine ligand 20 (CCL20), interleukin-21, C-C chemokine ligand 17 (CCL17), interleukin-1β (IL-1β), or any combination thereof; (b) one or more of: Pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2), or any combination thereof; or (c) one or more of: Granzyme B (GRAND B), fractalkine (CX3CL1), C-C motif chemokine ligand 24 (CCL24), interleukin-7 (IL-7), C-C chemokine ligand 20 (CCL20), interleukin-6 (IL-6), C-C chemokine ligand 17 (CCL17), interleukin-4 (IL-4), interleukin-17 (IL-17), interleukin-15 (IL-15), tumor necrosis factor alpha (TNFα), interleukin-1β (IL-1β), interferon gamma (INFγ), interleukin-12 (IL-12), or any combination thereof.

12. The method of claim 11, wherein the method further comprises:
a. determining a measurement of each of the first target biomarkers and second target biomarkers in a sample obtained from a subject suspected of having (i) multisystem inflammatory syndrome in children (MIS-C) or Kawasaki disease; (ii) MIS-C or SARS-CoV-2; or (iii) MIS-C or RSV;
b. calculating a relative intensity value for each first target biomarker and each second target biomarker in the panel based on the measurements in step a;
c. comparing the relative intensity values calculated in step b) to reference values for the target first and second biomarkers; and
d. assessing the subject for (i) MIS-C or Kawasaki disease; (ii) MIS-C or SARS-CoV-2; or (iii) MIS-C or RSV based on the comparison made in step c.

13. A device for assessing a subject suspected of having or having (i) multisystem inflammatory syndrome in children (MIS-C) or Kawasaki disease, (ii) MIS-C or Severe Acute Respiratory Syndrome Coronavirus 2 (SARS-CoV-2), or (iii) MIS-C or respiratory syncytial virus (RSV), the device comprising:
a. a measuring unit for determining a relative intensity of each of a plurality of first target biomarkers in a sample obtained from a subject suspected of having (i) MIS-C or Kawasaki disease, wherein the measuring unit comprises a detection system for detecting and measuring an amount of each of the plurality of first target biomarkers and further wherein the first target biomarkers comprise: Pentraxin-3 (PTX-3), Interleukin-10 (IL-10), Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-4 (IL-4), interleukin-17 (IL-17), chemokine ligand 5 (CCL5), tumor necrosis factor alpha (TNFα), interleukin-13 (IL-13), interferon gamma (INFγ), interleukin-12 (IL-12), and immunoregulatory alpha globulin (IRA); (ii) MIS-C or SARS-CoV-2, wherein the measuring unit comprises a detection system for detecting and measuring an amount of each of the plurality of first target biomarkers and further wherein the first target biomarkers comprise: Interleukin-33 (IL-33), C-X-C motif chemokine ligand 10 (CXCL10), Interleukin-27 (IL-27), C-C motif chemokine ligand 24 (CCL24), interleukin-6 (IL-6), interleukin-17 (IL-17), interferon gamma (INFγ), immunoregulatory alpha globulin (IRA), Marapsin, interleukin-2 receptor alpha chain (CD25), Heat Shock Protein 70 (HSP70), and interleukin-23 (IL-23); or (iii) MIS-C or RSV, wherein the measuring unit comprises a detection system for detecting and measuring an amount of each of the plurality of first target biomarkers and further wherein the first target biomarkers comprise: Granulocyte-Macrophage Colony-Stimulating Factor (GM-CSF), Pentraxin-3 (PTX-3), Marapsin, B-lymphocyte activation antigen B7 (B7-1), C-C Motif Chemokine Ligand 4 (CCL4), Triggering Receptor Expressed on Myeloid Cells (TREM), Heat Shock Protein 70 (HSP70), interleukin-33 (IL-33), C-X-C motif chemokine ligand 10 (CXCL10), interleukin-27 (IL-27), and interleukin-23 (IL-23); and
b. an evaluation unit operably linked to the measuring unit comprising a database with stored reference values for the first target biomarkers and a data processor comprising instructions for carrying out a comparison of the relative intensity of each first target biomarker to reference values for the target biomarkers and assessing the subject based on the comparison, said evaluation unit being capable of automatically receiving the relative intensity values for each of the biomarkers from the measuring unit.

14. The device of claim 13, wherein the first target biomarkers further comprise (a) one or more of B-lymphocyte activation antigen B7 (B7-1), C-X-C motif chemokine ligand 11 (CXCL11), interleukin-8 (IL-8), interleukin-7 (IL-7), interleukin-23 (IL-23), interleukin-2 (IL-2), interleukin-15 (IL-15), or any combination thereof; (b) one or more of: Pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2), or any combination thereof; or (c) one or more of: Granzyme B (GRAND B), fractalkine (CX3CL1), C-C motif chemokine ligand 24 (CCL24), interleukin-7 (IL-7), C-C chemokine ligand 20 (CCL20), interleukin-6 (IL-6), C-C chemokine ligand 17 (CCL17), interleukin-4 (IL-4), interleukin-17 (IL-17), interleukin-15 (IL-15), tumor necrosis factor alpha (TNFα), interleukin-1β (IL-1β), interferon gamma (INFγ), interleukin-12 (IL-12), or any combination thereof.

15. The device of claim 13, wherein:
a. the measuring unit determines a relative intensity of each of a plurality of first target biomarkers and second target biomarkers in the sample, wherein the measuring unit comprises a detection system for detecting and measuring an amount of each of the plurality of first target biomarkers and second target biomarkers and further wherein the second target biomarkers comprise (a) one or more of marapsin, fractalkine (CX3CL1), C-C chemokine ligand 20 (CCL20), interleukin-21, C-C chemokine ligand 17 (CCL17), interleukin-1β (IL-1β), or any combination thereof; (b) one or more of: Pentraxin-3 (PTX-3), interleukin-10 (IL-10), tumor necrosis factor alpha (TNFα), interleukin-13 (IL-13), fractalkine (CX3CL1), C-C chemokine ligand 17 (CCL17), interleukin-1β (IL-1β), Perforin (Perf), C-C Motif Chemokine Ligand 4 (CCL4), Granzyme B (GRAND B), C-X-C motif chemokine ligand 11 (CXCL11), interleukin-2 (IL-2), or any combination thereof; or (c) one or more of: Granzyme B (GRAND B), fractalkine (CX3CL1), C-C motif chemokine ligand 24 (CCL24), interleukin-7 (IL-7), C-C chemokine ligand 20 (CCL20), interleukin-6 (IL-6), C-C chemokine ligand 17 (CCL17), interleukin-4 (IL-4), interleukin-17 (IL-17), interleukin-15 (IL-15), tumor necrosis factor alpha (TNFα), interleukin-1β (IL-1β), interferon gamma (INFγ), interleukin-12 (IL-12), or any combination thereof; and
b. the evaluation unit comprises a database with stored reference values for the first target biomarkers and second target biomarkers and a data processor comprising instructions for carrying out a comparison of the relative intensity of each first target biomarker and each second target biomarker to reference values for the first and second target biomarkers and assessing the subject based on the comparison.

16. The device of claim 15, wherein the detection system comprises at least one detection agent capable of specifically detecting each of the first target biomarkers, the second target biomarkers, or both the first target biomarkers and the second target biomarkers.

17. The device of claim 16, wherein the detection agent is an antibody or antibody binding fragment thereof.
